(19) United States
(12) Patent Application Publication
(10) Pub. No.: US 2010/0170007 A1
(43) Pub. Date: Jul. 1, 2010

(54) GENES CONTROLLING WINTER DORMANCY IN PERENNIALS AND USES THEREOF

(75) Inventors: Douglas G. Bielenberg, Clemson, SC (US); Gregory L. Reighard, Clemson, SC (US); Albert G. Abbot, Clemson, SC (US)

Correspondence Address: Ballard Spahr LLP, SUITE 1000, 999 PEACHTREE STREET, ATLANTA, GA, 30309-3915, US

(73) Assignee: CLEMSON UNIVERSITY RESEARCH FOUNDATION, Clemson, SC (US)

(21) Appl. No.: 11/910,180

(22) PCT Filed: Mar. 28, 2006
(86) PCT No.: PCT/US06/11310

§ 371 Date: Mar. 16, 2010

Related U.S. Application Data
(60) Provisional application No. 60/665,694, filed on Mar. 28, 2005.
(60) Provisional application No. 60/777,628, filed on Feb. 28, 2006.

(51) Int. Cl. C12N 15/82; C12N 15/29; C07K 14/415; C12N 5/10; A01H 5/00

(52) U.S. Cl. 800/290; 536/23.6; 530/370; 435/320.1; 435/419; 800/298

(57) ABSTRACT

Provided herein are compositions and methods for modulating winter dormancy in a perennial plant using the herein disclosed winter dormancy genes and variants and homologues thereof.

### CROSS-REFERENCE TO RELATED APPLICATIONS

This application claims benefit of U.S. Provisional Application No. 60/665,694, filed Mar. 28, 2005, and U.S. Provisional Application No. 60/777,628, filed Feb. 28, 2006, which are hereby incorporated herein by reference in their entirety.

### STATEMENT REGARDING FEDERALLY SPONSORED RESEARCH

This invention was made with government support under Grant CSREES/USDA SC-2003055 awarded by the USDA. The government has certain rights in the invention.

### BACKGROUND

Woody perennials include those bushes and trees that keep their “woody” branching system above the ground alive during the cold winter months and usually go dormant until spring. Woody perennials such as trees used in the forestry industry for lumber, pulp, and biomass as well as fruit and nut trees are important for both their ecological and economic impact.

Two naturally occurring woody perennial mutants have been discovered that fail to cease growth and enter winter dormancy. The best described of these species, the Evergrowing (EVG) peach is believed to have arisen in Mexico, where killing frosts do not occur. In Mexico, terminal growth on EVG trees is continuous under the favorable environmental conditions, and the leaves are retained until they are lost to drought and/or disease. When grown at more northern latitudes, the EVG peach does not appear to respond to winter dormancy cues, exhibiting persistent growth and a lack of leaf abscission at the onset of short days and low temperatures in the fall. This behavior continues until these tissues are killed by freezing temperatures. Additionally, the frost hardiness of EVG trees has been found to be roughly half that of wild-type dormant trees. For example, EVG trees show some cold acclimation and accumulation of bark storage proteins and dehydrins, however, this occurs later in the fall and to a lesser degree than in wild type trees.

Formation of crosses of EVG (non-dormant) trees with different wild-type dormant trees has suggested that the EVG phenotype is controlled by a single recessive nuclear gene (Rodrigues J., et al. J Am Soc Hort Sci. 119:789-792). However, the specific genetic differentiation between the naturally occurring mutant EVG peach and wild-type dormant species that can account for the lack of response to winter dormancy cues in the EVG peach has not previously been determined.

### SUMMARY

In accordance with the purpose of this invention, as embodied and broadly described herein, this invention relates to genes for modulating winter dormancy in a perennial and uses thereof.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows fine genetic map and the peach contigs developed in the evergreen gene locus. BAC PpN018F12 is depicted in bold. The AFLP marker EAT/MCAC and the SSR marker pchgms40 flank the evg mutation

FIG. 2 shows gene map of 70.5 kb region of the EVG locus that showing relative locations and sizes of genes. For gene descriptions see text and Table 1. Large block arrows represent the sequence strand of the putative genes. Small inverted triangles indicate the location of the genetic markers that were mapped to positions flanking the EVG trait in the F2 mapping population.

FIG. 3 shows structural arrangement of MADS-box containing fragments within the EVG gene region (Bielenberg et al., 2004). A) Arrangement of the MADS-box containing fragments within the BAC contig spanning the EVG region. Letters refer to fragments identified in B. Missing bands are contiguous to one another in the wild-type and appear to be affected by one large deletion event. B). The MADS-box was hybridized with Southern blots of HindIII digested DNA from wild type and evg genomes and BACs PpN089G02, PpN018F12, and PpN018G07.

FIG. 4 shows amplification products from the SP6 ends of BACs PpN089G02 (A) and PpN018G07 (B) were labeled with ³²P-dCTP and hybridized with Southern blots containing HindIII digested DNA from wild type and evg genomes and BACs PpN089G02, PpN018F12, and PpN018G07. No polymorphism exists between the mutant and wild-type genomes at either position in the BAC contig. (Bielenberg et al., 2004).

FIG. 5 shows northern hybridization of wild-type and evg mutant total RNA with a probe amplified from the predicted coding region of the CaBP putative gene (see Table 1) in the EVG region. Twenty ug of total RNA isolated from June sampled shoot terminals was loaded into each lane of the gel, the right side image is the EtBr stained total RNA with prominent rRNA bands from both the. Left side image is the autoradiogram result following a 60 d exposure to the hybridized membrane.

FIG. 6 shows dot matrix representation of the 132 kb sequenced EVG region comparing the EVG sequence to itself along the entire length of the sequence with a floating window (‘Dotter’, Sonnhammer and Durbin, 1995). Lines parallel to but offset from the X=Y (100% match) central line represent regions of high similarity (repeats) that are duplicated within the region in the same orientation. Lines perpendicular to and offset from the X=Y line (100% match) represent regions of high similarity (repeats) that are duplicated in the region, but in an inverted orientation. Horizontal or vertical distance between lines represents distance between the similar regions. The coordinates of the regions can be determined from the X and Y axes.

FIG. 7 shows enlarged view of region ‘D’ in FIG. 6 highlighting a 4000 bp tandem repeat in the EVG region. X and Y axes are scaled in the enlarged image.

FIG. 8 shows enlarged view of region ‘I’ in FIG. 6 highlighting an approximately 200 bp inverted repeat in the EVG region. X and Y axes are scaled in the enlarged image.

FIG. 9 shows the HindIII subclone 18HB09 of BAC PpN018F12 labeled with ³²P-dCTP and hybridized with Southern blots containing HindIII digested DNA from wild type and evg genomes and BACs PpN089G02, PpN018F12, and PpN018G07. The restriction fragment size polymorphism detected in the mutant genomic relative to the wild-type genomic DNA was interpreted as representing the breakpoint of one border of the deletion detected in the mutant genome.

FIG. 10 shows the non-dormant evergrowing mutation increases biomass accumulation. A. Trunk cross-sectional area of wild-type (solid) and evergrowing mutant (open) F2 sibling trees after two years of growth in the field (Error marks are S.E.). B. Photograph of representative wild-type (L) and mutant (R) trees in the field. A letter-sized notepad lies between the trees in the photograph.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

By “inhibit” or other forms of inhibit means to hinder or restrain a particular characteristic. It is understood that this is typically in relation to some standard or expected value, in other words it is relative, but that it is not always necessary for the standard or relative value to be referred to. For example, “inhibits growth” means hindering or restraining the amount of growth that takes place relative to a standard or a control.

By “prevent” or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. It is understood that where reduce, inhibit or prevent are used, unless specifically indicated otherwise, the use of the other two words is also expressly disclosed. Thus, if inhibits growth is disclosed, then reduces and prevents growth are also disclosed.

By “reduce” or other forms of reduce means lowering of an event or characteristic. It is understood that this is typically in relation to some standard or expected value, in other words it is relative, but that it is not always necessary for the standard or relative value to be referred to. For example, “reduces phosphorylation” means lowering the amount of phosphorylation that takes place relative to a standard or a control.

As used herein, “modulate” or “modulating” can refer to an increase or decrease in an activity. This can include but is not limited to the inhibition or promotion of an activity, condition, disease, or response or other biological parameter.

As used herein, “polypeptide” can refer to a molecular chain of amino acids and does not necessarily indicate a specific length of the product. Thus, peptides, oligopeptides and proteins can be included within the definition of polypeptide. This term is can also include polypeptides that have been subjected to post-expression modifications such as, for example, glycosylations, acetylations, phosphorylations and the like.

As used herein, “protein” can refer to any molecular chain of amino acids that is capable to interacting structurally, enzymatically or otherwise with other proteins, polypeptides or any other organic or inorganic molecule.

As used herein, “fragment” in reference to a protein or polypeptide can refer to an amino acid sequence of that protein that is shorter than the entire protein, but comprising at least about 25 consecutive amino acids of the full polypeptide. When used to refer to a nucleic acid (e.g., cDNA), the term can be used herein to refer to a portion of the instant nucleic acid that has been constructed artificially or by cleaving a natural product into multiple pieces.

As used herein, “ortholog” can refer to a nucleotide or polypeptide sequence with similar function to a nucleotide or polypeptide sequence in an evolutionarily related species. Loci in two species are said to be “orthologs” when they have arisen from the same locus of their common ancestor. Orthologous polynucleotide sequences at loci in different species that are sufficiently similar to each other in their nucleotide sequences to suggest that they originated from a common ancestral sequence. Orthologous sequences arise when a lineage splits into two species, rather than when a sequence is duplicated within a genome. Proteins that are orthologs of each other are encoded by genes of two different species, and the genes are said to be orthologs.

As used herein, “homolog” can refer to two nucleotide or polypeptide sequences that differ from each other by substitutions that do not effect the overall functioning of the polypeptide. For example, when considering polypeptide sequences, homologues can include polypeptides having substitution of one amino acid at a given position in the sequence for another amino acid of the same class (e.g., amino acids that share characteristics of hydrophobicity, charge, pK or other conformational or chemical properties, e.g., valine for leucine, arginine for lysine). Homologues can also include polypeptides and nucleotide sequences including one or more substitutions, deletions, or insertions, located at positions of the sequence that do not alter the conformation or folding of the polypeptide to the extent that the biological activity of the polypeptide is destroyed. Examples of possible homologues include polypeptide sequences including substitution of one non-polar (hydrophobic) residue such as isoleucine, valine, leucine or methionine for one another; the substitution of one polar (hydrophilic) residue for another such as between arginine and lysine, between glutamine and asparagine, or between threonine and serine; the substitution of one basic residue such as lysine, arginine or histidine for another; the substitution of one acidic residue, such as aspartic acid or glutamic acid for the other; or the use of a chemically derivatized residue in place of a non-derivatized residue, as long as the homolog polypeptide displays substantially similar biological activity to the reference polypeptide.

As used herein, “analog” can refer to a non-natural molecule substantially similar to either the entire reference protein or polypeptide, or a fragment or allelic variant thereof, and having substantially the same or superior biological activity. The term “analog” can include derivatives (e.g., chemical derivatives, as defined above) of the biologically active polypeptide, as well as its fragments, homologs, orthologs, and allelic variants, which derivatives exhibit a qualitatively similar agonist or antagonist effect to that of the unmodified polypeptide.

As used herein, “allele” of a polypeptide can refer to a polypeptide sequence containing a naturally-occurring sequence variation relative to the polypeptide sequence of the reference polypeptide. Similarly, an allele of a polynucleotide encoding the polypeptide can be a polynucleotide containing a sequence variation relative to the reference polynucleotide sequence encoding the reference polypeptide, where the allele of the polynucleotide encoding the polypeptide encodes an allelic form of the polypeptide.

As used herein, “operably linked” can refer to a situation wherein the components described are in a relationship permitting them to function in their intended manner. For instance, a control sequence “operably linked” to a coding sequence is ligated in such a manner that expression of the coding sequence is achieved under conditions compatible with the control sequence.

A “coding sequence” can be a polynucleotide sequence that is transcribed into mRNA and translated into a polypeptide when placed under the control of (e.g., operably linked to) appropriate regulatory sequences. The boundaries of the coding sequence can be determined by a translation start codon at the 5′-terminus and a translation stop codon at the 3′-terminus. Such boundaries can be naturally-occurring, or can be introduced into or added to the polynucleotide sequence by methods known in the art. A coding sequence can include, but is not limited to, genomic DNA, mRNA, cDNA, and recombinant polynucleotide sequences.

As used herein, “sequence identity” can refer to the subunit sequence similarity between two polymeric molecules: for example, the sequence similarity between two polynucleotides or two polypeptides. When a subunit position in both of the two molecules is occupied by the same monomeric subunit, then they are identical at that position. The identity between two sequences is a direct function of the number of matching or identical positions. For example, if half of the positions in two peptide or compound sequences are identical, then the two sequences are 50% identical. The identity between two sequences can be a direct function of the number of matching or identical positions. Thus, if a portion of the reference sequence is deleted in a particular peptide, that deleted section is not counted for purposes of calculating sequence identity. For example, when comparing a first polymer including monomers R₁R₂R₃R₄R₅R₆ with another polymer including monomers R₁R₂R₃R₄R₆, the two polymers can be considered to have 5 out of 6 positions in common, and therefore could described as sharing 83.3% sequence identity.

As used herein, an antibody “specific for” a polypeptide, or that “specifically binds” a polypeptide, can be considered to include a material that binds with substantially higher affinity to that polypeptide than to an unrelated polypeptide. In addition, an antibody specific for a particular polypeptide also can have specificity for a related polypeptide. For example, an antibody specific for a polypeptide derived from a particular peach tree, can specifically bind another related polypeptide from a different cultivar.

As used herein, “gene” or “genes” can be used to mean nucleic acid sequences (including both RNA and DNA) that encode genetic information for the synthesis of a whole RNA, a whole protein, or any portion of such whole RNA or whole protein. Genes that are not part of a particular plant's genome can be referred to as “foreign genes” and genes that are a part of a particular plant's genome can be referred to as “endogenous genes.” The term “gene product” can refer to RNAs or proteins that are encoded by the gene. “Foreign gene products” can be RNA or proteins encoded by foreign genes and “endogenous gene products” can be RNA or proteins encoded by endogenous genes.

As used herein, “nucleic acid” can refer to natural and synthetic linear and sequential arrays of nucleotides and nucleosides, for example cDNA, genomic DNA, mRNA, RNA, oligonucleotides, oligonucleosides and derivatives thereof. For ease of discussion, such nucleic acids may be collectively referred to as herein a “constructs,” “plasmids,” or “vectors.” Representative examples of the nucleic acids of the present invention include bacterial plasmid vectors such as expression, cloning, cosmid and transformation vectors (for example, pBR322, lambda and the like), plant viral vectors (modified TMV, tobamovirus, and the like), and synthetic oligonucleotides like chemically synthesized DNA or RNA.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular vector is disclosed and discussed and a number of vector components including the promoters are discussed, each and every combination and permutation of promoters and other vector components and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Provided herein are genes responsible for winter dormancy in a perennial plant. Thus, also provided are compositions and methods for modulating winter dormancy in a perennial plant using the herein disclosed winter dormancy genes and variants and homologues thereof. Modulating winter dormancy in a perennial plant has many uses, some of which are disclosed herein. For example, winter dormancy can be inhibited to increase the growing season of the plant. Also, winter dormancy can be inhibited to increase the biomass of a plant expressing the gene(s). Alternatively, winter dormancy can be activated to prevent winter damage. The specific advantages and uses of the herein disclosed compositions and methods are not meant to be limiting and can be used or adapted for use for other purposes, systems, or outcomes.

### 1. Perennial Plants

A perennial plant or “perennial” is a plant that produces flowers and seeds more than once in its lifespan, and therefore lives for more than one year. As used herein, this term applies to all plants which flowers and produces seeds more than once. A plant that flowers and produces seeds only once in its lifetime is called a “monocarp”. These include annual plants, which flower in their first living year, then die, or biennial plants, which flower in their second season. Some monocarp plants can live for many years before flowering (and dying) as bamboo and agave.

Herbaceous perennials are plants that do not form permanent woody tissue. In warmer and more clement climates they may grow continuously. In seasonal climates, their growth pattern is adapted to the growing season. In cooler temperate regions they generally grow and bloom during the warm part of the year, and the foliage dies back every winter. Regrowth is from their existing tissue or root-stock rather than from seed, as with annuals and biennials. In some cases, these perennials may retain their foliage all year round, even in seasonal climates. Herbaceous perennials that retain their foliage all year round may be called evergreen perennials. Others are called deciduous. Woody perennials (ie. trees and shrubs) retain their woody structure permanently, but may lose their foliage in seasonal climates.

Perennial plants live more than 2 years and are grouped into two categories: herbaceous perennials and woody perennials. Herbaceous perennials have soft, nonwoody stems that generally die back to the ground each winter. New stems grow from the plant's crown each spring. Trees and shrubs, on the other hand, have woody stems that withstand cold winter temperatures. They are referred to as woody perennials. There are many perennial plants important to human food production including many herbs, shrubs, and trees. There are other commercial and ecological uses for perennial plants, including that of biomass or bioenergy.

### 2. Biomass

Biomass is a scientific term for living matter, but the word biomass is also used to denote products derived from living organisms—wood from trees, harvested grasses, plant parts and residues such as twigs, stems and leaves, as well as aquatic plants and animal wastes. All the Earth's biomass exists in a thin surface layer called the biosphere. This represents only a tiny fraction of the total mass of the Earth, but in human terms it is an enormous store of energy—as fuel and as food. More importantly, it is a store which is being replenished continually. The source which supplies the energy is of course the Sun, and although only a tiny fraction of the solar energy reaching the Earth each year is converted into biomass, it is nevertheless equivalent to over five times total world energy consumption.

Biomass energy or “bioenergy” includes any type of energy, including any solid, liquid or gaseous fuel, or any electric power or useful chemical product derived from organic matter, whether directly from plants or indirectly from plant-derived industrial, commercial, or urban wastes, or agricultural and forestry residues. Thus bioenergy can be derived from a wide range of raw materials and produced in a variety of ways. Because of the wide range of potential feedstocks and the variety of technologies to produce them and process them, bioenergy is usually considered as a series of many different feedstock/technology combinations. The term “biopower” describes biomass power systems that use biomass feedstocks instead of the usual fossil fuels (natural gas or coal) to produce electricity, and the term “biofuel” is used mostly for liquid transportation fuels which substitute for petroleum products such as gasoline or diesel.

Energy crops, also called “bioenergy crops”, are fast-growing crops that are grown for the specific purpose of producing energy (electricity or liquid fuels) from all or part of the resulting plant. The plants that have been selected by the U.S. Department of Energy for further development as energy crops are mostly perennials such as switchgrass, willow and poplar. They were selected for their advantageous environmental qualities such as erosion control, soil organic matter build-up and reduced fertilizer and pesticide requirements. There are many other perennial plant species which could be used for energy crops.

### 3. Winter Dormancy

In northern areas, low temperature is the major environmental factor limiting the productivity and the geographical distribution of perennial plants. Low temperature decreases biosynthetic activity of plants, disturbs the normal function of physiological processes and may result in permanent injuries that finally bring about death. Adaptation to seasonal changes in temperature is a precondition for woody plant life in temperate and boreal vegetation zones. The annual process of cold acclimation involves structural and metabolic adjustments that result in a transition from a lower to a higher level of cold hardiness. The ultimate survival of woody plants is dependent on not only the maximal capacity of cold hardening, but also on the timing and rate of both cold acclimation and deacclimation, the stability of cold hardiness, and the ability to reacclimate after unseasonably warm periods. Hence, the successful performance of a woody species in a particular locality implies synchronization of the annual development of cold hardiness with the seasonal temperature changes.

The seasonal cold acclimation of woody plants native to the temperate zones is a three-stage process. The first stage is strongly affected by photoperiod. In many woody plants short days induce growth cessation, which is a prerequisite for cold acclimation. The first stage, during which abundant organic substances are stored, depends mainly on photosynthesis and proceeds at relatively warm temperatures in autumn. Cells in the first stage of acclimation can survive temperatures well below 0° C., but they are not fully hardened. The second stage of cold acclimation is induced by low temperature, especially subzero temperatures. During this stage plants undergo metabolic and/or structural changes, which lead to a considerable degree of cold hardiness. In many woody taxa, the maximum level of cold hardiness is obtained first after an exposure to low freezing temperatures (−30° to −50° C.). This can be defined as the third phase of cold hardening.

Thus, low temperature and shortening photoperiod are the two major factors triggering cold acclimation in woody plants. The sequence of these environmental cues is essential: short days should precede low temperatures for the whole acclimation capacity to be manifested. Furthermore, photoperiodic effects on growth and development can be modified by temperature. Also water availability, mineral nutrition, and plant age can bring about alterations in the cold acclimation process.

### 4. Winter Dormancy Genes

Mutants that fail to cease growth and enter dormancy under dormancy-inducing conditions have been described in only two tree species, Corylus avellana L. (Hazel) (Thompson et al. 1985) and Prunus persica (L.) Batsch (Peach) (Rodriguez et al. 1994). The Evergrowing (EVG) peach is the best described of the mutants. The EVG peach mutant does not set terminal buds, cease new leaf growth, or enter into a dormant resting phase in response to winter conditions. The EVG mutation segregates as a single recessive gene. A local molecular genetic linkage map around EVG was previously developed using amplified fragment length polymorphism (AFLP) and simple sequence repeat (SSR) markers, and a bacterial artificial chromosome (BAC) contig that contains the EVG mutation was assembled. As disclosed herein, a MADS box coding open reading frame (ORF) was found in a BAC of this contig and used as a probe. The probe detected a polymorphism between the wild-type and mutant genomes, which was indicative of a deletion in EVG peach. This region is referred to herein as the EVG locus or deletion region. This BAC was completely sequenced (SEQ ID NO:24), and sequence analysis thereof predicted a number of putative genes. The EVG gene region contained six putative MADS-box transcription factor sequences, and the deletion in EVG affected at least four of these. Additionally, there was a Ca²⁺ binding protein.

As disclosed herein, the genes of EVG locus of the peach tree have homologues in other perennial trees. Thus, this locus is generically referred to herein as evergreen locus. Provided herein is a method of modulating the growing season of a perennial tree, comprising deleting, disrupting, or suppressing one or more target genes in the evergreen locus. Thus, the evergreen locus can be an allelic variant of the EVG peach locus.

### 5. Gene Deletion or Disruption

The disclosed method can comprise targeted gene deletion, disruption, or modification of one or more target genes in the evergreen locus in any perennial plant that can undergo these events. Gene deletion, modification and disruption refer to the methods, techniques, and compositions that surround the selective removal or alteration of a gene or stretch of chromosome in an organism, such as a perennial plant, in a way that propagates the modification through the germ line of the plant. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are known and disclosed herein.

For example, gene silencing in perennials is described in Broothaerts, Keulemans & Van Nerum, 2004, Chao, 2002, Hily, Scorza, Malinowski, Zawadzka & Ravelonandro, 2004, Mlotshwa, Voinnet, Mette, Matzke, Vaucheret, Ding, Pruss & Vance, 2002, which are incorporated herein by reference in their entirety for these teachings.

For example, a vector can be designed for homologous recombination based on the disclosed nucleic acids SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

i. Functional Nucleic Acids

Gene disruption of one or more target genes in the evergreen locus can also comprise using a functional nucleic acid. Functional nucleic acids can be either transiently expressed by a target cell or integrated into the target cell genome. Disclosed herein are any functional nucleic acids designed based on the sequences for the herein disclosed winter dormancy genes set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8. In one aspect, the functional nucleic acid is designed to be specific for the herein disclosed winter dormancy genes. In another aspect, the functional nucleic acid is designed to disrupt gene expression of homologous and orthologous genes. Specificity of the functional nucleic acid can be chosen based on the whether the target sequence is conserved among species or protein families (e.g., MADS-boxes) or whether the target sequence is a divergent region specific to the gene target.

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹². A representative sample of methods and techniques which aid in the design and use of antisense molecules can be found in U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with K_{d}'s from the target molecule of less than 10-12 M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule (U.S. Pat. No. 5,543,293). It is preferred that the aptamer have a K_{d} with the target molecule at least 10, 100, 1000, 10,000, or 100,000 fold lower than the K_{d} with a background binding molecule. It is preferred when doing the comparison for a polypeptide for example, that the background molecule be a different polypeptide. Representative examples of how to make and use aptamers to bind a variety of different target molecules can be found in U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203; International Patent Application Nos. WO 9858058 by Ludwig and Sproat, WO 9858057 by Ludwig and Sproat, and WO 9718312 by Ludwig and Sproat) hairpin ribozymes (for example, U.S. Pat. Nos. 5,631,115, 5,646,031, 5,683,902, 5,712,384, 5,856,188, 5,866,701, 5,869,339, and 6,022,962), and tetrahymena ribozymes (for example, U.S. Pat. Nos. 5,595,873 and 5,652,107). There are also a number of ribozymes that are not found in natural systems, but which have been engineered to catalyze specific reactions de novo (for example, U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a K_{d} less than 10-6, 10-8, 10-10, or 10-12. Representative examples of how to make and use triplex forming molecules to bind a variety of different target molecules can be found in U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). This silencing was originally observed with the addition of double stranded RNA (dsRNA) (Fire, A., et al. (1998) Nature, 391:806-11; Napoli, C., et al. (1990) Plant Cell 2:279-89; Hannon, G. J. (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200; Bernstein, E., et al. (2001) Nature, 409:363-6; Hammond, S. M., et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al. (2001) Nature, 411:494 498) (Ui-Tei, K., et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAs (shRNAs). Kits for the production of vectors comprising shRNA are available, such as, for example, Imgenex's GENBSUPPRESSOR™ Construction Kits and Invitrogen's BLOCK-IT™ inducible RNAi plasmid and lentivirus vectors. Disclosed herein are any shRNA designed as described above based on the sequences for the herein disclosed inflammatory mediators.

RNAi can be designed based on the conserved domains specific to a class of genes, for example the MADS-box domain, the K-box domain, or the conserved C-terminus domain. For example, MADS box structure, evolution and conservation citations have been described in Alvarez-Buylla, Liljegren, Pelaz, Gold, Burgeff, Ditta, Vergara-Silva & Yanofsky, 2000, Aswath & Kim, 2005, Aswath, Mo, Kim & Kim, 2004, Brill & Watson, 2004, Garcia-Maroto, Carmona, Garrido, Vilches-Ferron, Rodriguez-Ruiz & Alonso, 2003, Johansen, Pedersen, Skipper & Frederiksen, 2002, Kim, Mizuno & Fujimura, 2002, Lohmann & Weigel, 2002, Parenicova, de Folter, Kieffer, Horner, Favalli, Busscher, Cook, Ingram, Kater, Davies, Angenent & Colombo, 2003, Prakash & Kumar, 2002, Ratcliffe, Kumimoto, Wong & Riechmann, 2003, Rosin, Hart, Van Onckelen & Hannapel, 2003, van der Linden, Vosman & Smulders, 2002, Vergara-Silva, Martinez-Castilla & Alvarez-Buylla, 2000, Yao, Dong, Kvarnheden & Morris, 1999, Yao, Dong & Morris, 2001, which are incorporated herein by reference in their entirety for these teachings.

### 6. Transgenic Expression

It can also be desirable to activate winter dormancy in a tree in order to avoid winter injury. Thus, also provided is a method of modulating winter dormancy of a perennial tree, comprising administering to the tree a nucleic acid comprising one or more target genes in the evergreen locus. The nucleic acid preferably has all appropriate sequences for expression of the nucleic acid, as known in the art, to functionally encode, i.e., allow the nucleic acid to be expressed. The nucleic acid can include, for example, expression control sequences, such as an enhancer, and necessary information processing sites, such as ribosome binding sites, RNA splice sites, polyadenylation sites, and transcriptional terminator sequences. As an example, the nucleic acid encoding the winter dormancy gene can be operably linked to a non-native or modified expression control sequence (e.g., promoter).

With the availability and on going development of plant transformation techniques, most commercially important plant species can now be genetically modified to express a variety of recombinant proteins. Such transformation techniques include, for example, the Agrobacterium vector system, which involves infection of the plant tissue with a bacterium (Agrobacterium) into which the foreign gene has been inserted. A number of methods for transforming plant cells with Agrobacterium are well known (Klee et al., Annu. Rev. Plant Physiol. (1987) 38:467-486; Schell and Vasil Academic Publishers, San Diego, Calif. (1989) p. 2-25; and Gatenby (1989) in Plant Biotechnology, eds. Kung, S. and Arntzen, C. J., Butterworth Publishers, Boston, Mass. p. 93-112), all of which are hereby incorporated herein by reference for their teaching of plant transforming methods.

The biolistic or particle gun method, which permits genetic material to be delivered directly into intact cells or tissues by bombarding regeneratable tissues, such as meristems or embryogenic callus, with DNA-coated microparticles has contributed to plant transformation simplicity and efficiency. The microparticles penetrate the plant cells and act as inert carriers of a genetic material to be introduced therein. Microprojectile bombardment of embryogenic suspension cultures has proven successful for the production of transgenic plants of a variety of species. Various parameters that influence DNA delivery by particle bombardment have been defined (Klein et al., Bio/Technology (1998) 6:559-563; McCabe et al., Bio/Technology (1998) 6:923-926; and Sanford, Physiol. Plant. (1990) 79:206-209), all of which are hereby incorporated herein by reference for their teaching of biolistic and particle gun methods.

Micropipette systems are also used for the delivery of foreign DNA into plants via microinjection (Neuhaus et al., Theor. Appl. Genet. (1987) 75:30-36; and Neuhaus and Spangenberg, Physiol. Plant. (1990) 79:213-217), all of which are hereby incorporated herein by reference for their teaching of micropipette systems.

Other techniques developed to introduce foreign genes into plants include direct DNA uptake by plant tissue, or plant cell protoplasts (Schell and Vasil (1987) Academic Publishers, San Diego, Calif. p. 52-68; and Toriyama et al., Bio/Technology (1988) 6:1072-1074) or by germinating pollen (Chapman, Mantell and Daniels (1985) W. Longman, London, p. 197-209; and Ohta, Proc. Natl. Acad. Sci. USA (1986) 83:715-719), all of which are hereby incorporated herein by reference for their teaching of plant transforming methods.

DNA uptake induced by brief electric shock of plant cells has also been described (Zhang et al., Plant. Cell. Rep. (1988) 7:379-384 and Fromm et al., Nature (1986) 319:791-793), all of which are hereby incorporated herein by reference for their teaching of plant transforming methods.

In addition, virus mediated plant transformation has also been extensively described. Transformation of plants using plant viruses is described, for example, in U.S. Pat. No. 4,855,237 (BGV), EP-A 67,553 (TMV), Japanese Published Application No. 63-14693, EPA 194,809, EPA 278,667, and Gluzman et al., (1988) Communications in Molecular Biology: Viral Vectors, Cold Spring Harbor Laboratory, New York, pp. 172-189. Pseudovirus particles for use in expressing foreign DNA in many hosts, including plants, have also been described, for reference, see, for example WO 87/06261. All preceding references are hereby incorporated herein by reference for their teaching of plant transforming methods.

The production of recombinant proteins and peptides in plants has been investigated using a variety of approaches including transcriptional fusions using a strong constitutive plant promoter (e.g., from cauliflower mosaic virus, Sijmons et al., Bio/Technology (1990) 8:217-221); transcriptional fusions with organ specific promoter sequences (Radke et al., Theoret. Appl. Genet. (1988) 75:685-694); and translational fusions which require subsequent cleavage of a recombinant protein (Vanderkerckove et al., Bio/Technology (1989) 7:929-932). All preceding references are hereby incorporated herein by reference for their teaching of transcriptional fusions for production of recombinant proteins in plants.

The application of such genetic transformation techniques has allowed the incorporation of a variety of important genetic traits for crop improvement and also for the biotechnological production of extractable, valuable, foreign proteins including enzymes, vaccine proteins and antibodies.

Foreign proteins that have been successfully expressed in plant cells include proteins from bacteria (Fraley et al. Proc. Natl. Acad. Sci. U.S.A (1993) 80:4803-4807), animals (Misra and Gedamu, Theor. Appl. Genet. (1989) 78:161-168), fungi and other plant species (Fraley et al. Proc. Natl. Acad. Sci. U.S.A. (1983) 80:4803-4807). Some proteins, predominantly markers of DNA integration, have been expressed in specific cells and tissues including seeds (Sen Gupta-Gopalan et al. Proc. Natl. Acad. Sci. U.S.A. (1985) 82:3320-3324; Radke et al. Theor. Appl. Genet. (1988) 75:685-694).

The nucleic acid encoding the protein of interest can be introduced into a host cell in a form where the nucleic acid is stably incorporated into the genome of the host cell. One may also introduce the nucleic acid as part of a recombinant DNA sequence capable of replication and or expression in the host cell without the need to become integrated into the host chromosome.

The nucleic acid introduced into the plant cell may also comprise sequences for regulation of transcription which are recognized by the plant cell. The regulatory sequences can comprise one or more promoter(s) of plant or viral origin or obtained from Agrobacterium tumefaciens. Thus, the nucleic acid can comprise a constitutive promoter, for example the CaMV 35S, the double 35S, the Nos or OCS promoters, or promoters specific for certain tissues such as the grain or specific for certain phases of development of the plant. The nucleic acid can comprise promoters specific for seeds, such as the promoter of the gene for napin and for the acyl carrier protein (ACP) (EP-A-0,255,378), as well as the promoters of the AT2S genes of Arabidopsis thaliana, that is to say the PAT2S 1, PAT2S2, PAT2S3 and PAT2S4 promoters (Krebbers et al., Plant Physiol., 1988, vol. 87, pages 859-866). The nucleic acid can comprise\the cruciferin or phaseolin promoter or pGEA1 and pGEA6 of Arabidopsis, promoters of genes of the “em, Early Methionine labeled protein” type, which is strongly expressed during the phases of drying of the seed.

The introduction of a nucleic acid molecule(s) into the plant cell can be carried out in a stable manner either by transformation of the nuclear genome, or by transformation of the chloroplast genome of the plant cell, or by transformation of the mitochondrial genome.

For the transformation of the nuclear genome, conventional techniques may be used. All known means for introducing foreign DNA into plant cells may be used, for example Agrobacterium (e.g., Agrobacterium tumefaciens and Agrobacterium rhizogenes), electroporation, protoplast fusion, particle gun bombardment, or penetration of DNA into cells such as pollen, microspore, seed and immature embryo. Viral vectors such as the Gemini viruses or the satellite viruses may also be used as introducing means.

The introduction of the nucleic acid into the plant cell can also be carried out by the transformation of the mitochondrial or chloroplast genomes (see for example Carrer et al., Mol. Gen. Genet., 1993, 241, 49-56). Techniques for direct transformation of the chloroplasts or the mitochondria are known per se and may comprise introducing transformant DNA by the biolistic technique (Svab et al., P.N.A.S., 1990, 87, 8526-8530); integrating the transformant DNA by two homologous recombination events; and selectively removing copies of the wild-type genome during repeated cell divisions on selective medium.

Chimeric or transgenic plants can be generated from transformed explants, using techniques known per se. The term “transgenic plant” refers to a plant that contains genetic material, not found in a wild type plant of the same species, variety or cultivar. The genetic material may include a transgene, an insertional mutagenesis event, an activation tagging sequence, a mutated sequence, a homologous recombination event or a sequence modified by chimeraplasty. Typically, the foreign genetic material has been introduced into the plant by human manipulation.

A transgenic plant may contain an expression vector or cassette. The expression cassette typically comprises a polypeptide-encoding sequence operably linked (i.e., under regulatory control of) to appropriate inducible or constitutive regulatory sequences that allow for the expression of polypeptide. The expression cassette can be introduced into a plant by transformation or by breeding after transformation of a parent plant. A plant refers to a whole plant as well as to a plant part, such as seed, fruit, leaf, or root, plant tissue, plant cells or any other plant material, e.g., a plant explant, as well as to progeny thereof, and to in vitro systems that mimic biochemical or cellular components or processes in a cell.

Thus, provided is a recombinant perennial plant comprising a heterologous winter dormancy gene, wherein the gene has 70%, 75%, 80%, 85%, 90%, 95%, 100% sequence identity to the winter dormancy genes set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

### 7. Isolated Nucleic Acid

Also provided herein are isolated nucleic acids encoding winter dormancy genes. The nucleic acid can have the sequence set forth SEQ ID NO:1, SEQ ID NO:2, SEQ ID NO:3, SEQ ID NO:4, SEQ ID NO:5, SEQ ID NO:6, SEQ ID NO:7, or SEQ ID NO:8. The nucleic acid can hybridize to the nucleic acid sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8 under stringent conditions or other conditions as disclosed herein. The nucleic acid can comprise a sequence with at least 70%, 75%, 80%, 85%, 90%, 95% identity to the sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8. The nucleic acids can encode polypeptides that can comprise conservative mutations, deletions, substitutions, or additions.

Also provided is a nucleic acid encoding SEQ ID NO:1, SEQ ID NO:2, SEQ ID NO:3, SEQ ID NO:4, SEQ ID NO:5, SEQ ID NO:6, SEQ ID NO:7, or SEQ ID NO:8 functionally linked to an expression control sequence.

i. Nucleic Acids

There are a variety of molecules disclosed herein that are nucleic acid based, including for example the nucleic acids that encode, for example SEQ ID NOs: 1-8, or fragments thereof. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a. Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b. Sequences

There are a variety of sequences related to winter dormancy genes disclosed herein, for example SEQ ID NOs: 1-8. The sequences for the analogs and alleles of these genes, and splice variants and other types of variants, are available in a variety of protein and gene databases, including Genbank. Those sequences available at the time of filing this application at Genbank are herein incorporated by reference in their entireties as well as for individual subsequences contained therein. Genbank can be accessed at http://www.ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

c. Primers and Probes

Disclosed are compositions including primers and probes, which are capable of interacting with the disclosed nucleic acids, such as the SEQ ID NOs: 1-8 as disclosed herein.

For example, forward and reverse primers for amplifying MADS-1 are set forth in SEQ ID NO:10 and 11. For example, forward and reverse primers for amplifying MADS-2 are set forth in SEQ ID NO:12 and 13. For example, forward and reverse primers for amplifying an alternative variant of MADS-2 are set forth in SEQ ID NO:14 and 15. For example, forward and reverse primers for amplifying MADS-3 are set forth in SEQ ID NO:16 and 17. For example, forward and reverse primers for amplifying MADS-4 are set forth in SEQ ID NO:18 and 19. For example, forward and reverse primers for amplifying MADS-5 are set forth in SEQ ID NO:20 and 21. For example, forward and reverse primers for amplifying MADS-6 are set forth in SEQ ID NO:22 and 23.

In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the gene typically will be used to produce an amplified DNA product that contains a region of the gene or the complete gene. In general, typically the size of the product will be such that the size can be accurately determined to within 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

d. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

It is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

ii. Peptides

As disclosed herein there are numerous variants of the winter dormancy genes that are herein contemplated. In addition, to the known functional species variants, there are derivatives of the genes which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative Substitutions,
others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

iii. Sequence Similarities

It is understood that one way to define the variants and derivatives of the disclosed proteins herein is through defining the variants and derivatives in terms of homology/identity to specific known sequences. Those of skill in the art readily understand how to determine the homology of two proteins. For example, the homology can be calculated after aligning the two sequences so that the homology is at its highest level.

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

It is understood that there are numerous amino acid and peptide analogs which can be incorporated into the disclosed compositions. For example, there are numerous D amino acids or amino acids which have a different functional substituent than the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

### 8. Cells

Also provided herein is a cell comprising any of the herein provided nucleic acids or vectors. The cell can be any cell that can be transformed with a nucleic acid molecule provided herein. Host cells can be either untransformed cells or cells that are already transformed with at least one nucleic acid molecule (e.g., nucleic acid molecules encoding one or more proteins provided herein). Host cells provided herein either can be endogenously (i.e., naturally) capable of producing the proteins provided herein or can be capable of producing such as a result of engineering, such as by the methods provided herein. Cells provided herein can be any cell capable of producing at least one protein provided herein, and include bacterial, fungal (including yeast), parasite (including helminth, protozoa and ectoparasite), other insect, other animal and plant cells. Thus, the provided cell can be a bacterial, mycobacterial, fungal (e.g., yeast), helminth, insect or mammalian cell.

Thus, provided is a cell comprising a nucleic acid having the sequence set forth in SEQ ID NO: 1, 2, 3, 4, 6, 7, or 8. Also provided is a plant cell comprising a nucleic acid having the sequence set forth in SEQ ID NO: 1, 2, 3, 4, 5, 6, 7, or 8. Also provided is a mammalian cell comprising a nucleic acid having the sequence set forth in SEQ ID NO: 1, 2, 3, 4, 5, 6, 7, or 8. Also provided is a bacterial cell comprising a nucleic acid having the sequence set forth in SEQ ID NO: 1, 2, 3, 4, 5, 6, 7, or 8. Also provided is a yeast cell comprising a nucleic acid having the sequence set forth in SEQ ID NO: 1, 2, 3, 4, 5, 6, 7, or 8.

### 9. Product-by-Process

Provided herein is a perennial plant produced by any of the methods disclosed herein. Thus, provided is a plant produced by a process comprising deleting or disrupting one or more genes from the evergreen locus. Thus, provided is a plant produced by a process comprising administering to the plant a nucleic acid comprising one or more genes from the evergreen locus functionally linked to an expression control sequence.

### 10. Antibodies

Also provided herein is an antibody specific for any of the herein provided polypeptides. Thus, provided is an antibody specific for a polypeptide encoded by the nucleic acid set forth in SEQ ID NO: 1, 2, 3, 4, 5, 6, 7, or 8.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with the winter dormancy proteins. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

Administration of the antibodies can be done as disclosed herein. Nucleic acid approaches for antibody delivery also exist. The antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### 11. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

### 12. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved. Disclosed are computer readable mediums comprising the sequences and information regarding the sequences set forth herein.

### 13. Specific Embodiments

Provided herein is a method of modulating the growing season of a perennial tree, comprising deleting or suppressing one or more target genes in the evergreen locus. The perennial tree can be a poplar. The evergreen locus can be an allelic variant of the Evergrowing (EVG) peach locus. The growing season of a perennial tree can be increased. For example, the biomass of the perennial tree can be increased at least about 5%, 10%, 15%, 20%, 30%, 40%, 50%, 60% per year as compared to a control tree. The gene can be a MADS-box gene encoding a transcription factor. The gene can comprise a sequence having at least 70%, 75%, 80%, 85%, 90%, or 95% identity to the nucleic acid sequence set forth in SEQ ID NO:1, SEQ ID NO:2, SEQ ID NO:3, SEQ ID NO:4, SEQ ID NO:5, SEQ ID NO:6, or SEQ ID NO:7. The gene can encode a calcium binding protein. The gene can comprise a sequence having at least 70%, 75%, 80%, 85%, 90%, or 95% identity to the nucleic acid sequence set forth in SEQ ID NO:8. The gene can be deleted using homologous recombination. The can be suppressed using a functional nucleic acid. The functional nucleic acid can be an antisense, ribozyme, siRNA, or shRNA. The functional nucleic acid can comprise a sequence having at least 70%, 75%, 80%, 85%, 90%, or 95% identity to a portion of the nucleic acid sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

Also provided is a method of modulating winter dormancy of a perennial tree, comprising administering to the tree a nucleic acid comprising one or more target genes in the evergreen locus. The nucleic acid can be administered to the tree in an Agrobacterium vector. Winter dormancy can be prematurely activated in the tree. The nucleic acid can be operably linked to an expression control sequence. The expression control sequence can be an inducible promoter. The perennial tree can be a poplar.

Provided is a nucleic acid comprising the sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8. Also provided is a nucleic acid that hybridizes under stringent conditions to the nucleic acid sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8. Also provided is a nucleic acid comprising a sequence having at least 70%, 75%, 80%, 85%, 90%, or 95% identity to the nucleic acid sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8. Also provided is a polypeptide encoded by a nucleic acid sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or a conservative variant or fragment thereof. Also provided is a polypeptide having at least 70%, 75%, 80%, 85%, 90%, or 95% identity to a polypeptide encoded by a nucleic acid sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or a conservative variant or fragment thereof.

Also provided is a vector comprising a nucleic acid sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or a conservative variant or fragment thereof. The nucleic acid sequence can be operably linked to an inducible promoter.

Also provided is a method of making a transgenic organism comprising administering the nucleic acid sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or a conservative variant or fragment thereof.

Also provided is a method of making a transgenic organism comprising administering a vector comprising a nucleic acid sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or a conservative variant or fragment thereof.

### 14. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include the herein provided cells (e.g., seeds) for producing the herein disclosed transgenic plants, and the reagents for cultivating said plants.

### C. METHODS OF MAKING THE COMPOSITIONS

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert -butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process Claims for Making the Compositions

Disclosed are processes for making the compositions as well as making the intermediates leading to the compositions. For example, disclosed are nucleic acids in SEQ ID NOs: 1-8. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth in SEQ ID NO: 1, 2, 3, 4, 5, 6, or 7 and a sequence controlling the expression of the nucleic acid.

Also disclosed are nucleic acid molecules produced by the process comprising linking in an operative way a nucleic acid molecule comprising a sequence having 80% identity to a sequence set forth in SEQ ID NO: 1, 2, 3, 4, 5, 6, 7, or 8 and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence set forth in SEQ ID NO: 1, 2, 3, 4, 5, 6, 7, or 8 and a sequence controlling the expression of the nucleic acid.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are plants produced by the process of transfecting a cell within the plant or seed with any of the nucleic acid molecules disclosed herein.

### D. METHODS OF USING THE COMPOSITIONS

1. Methods of Using the Compositions as Research Tools

The disclosed compositions can be used in a variety of ways as research tools. For example, the disclosed compositions can be used as either reagents in micro arrays or as reagents to probe or analyze existing microarrays. The disclosed compositions can be used in any known method for isolating or identifying single nucleotide polymorphisms. The compositions can also be used in any method for determining allelic analysis. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Sequencing and Analysis of the Peach EVG Locus

i. Materials and Methods

Genomic DNA Isolation: Peach leaves for DNA extraction were obtained from individuals of an F₂ mapping population individuals and from moderate chilling cultivars (Musser Fruit Research Center, Clemson University, Clemson, S.C., USA). Leaves were weighed as 1.0 g fresh weight samples, wrapped in aluminum foil, frozen in liquid N₂, and stored at −80° C. Total DNA was isolated from the frozen leaves using a CTAB (hexadecyltrimethylammonium bromide) extraction buffer protocol modified from Doyle and Doyle (1990).

SSR marker Identification: New SSR loci were identified from subclones of BAC PpN018F12 were located, sequenced, primers designed, and mapped in the evg mapping population using the methods detailed in Wang et al. (2002).

BAC DNA Isolation: All BAC clones used in this study were obtained from a genomic library of the peach rootstock Nemared (wild-type dormant) (Georgi et al. 2002). BAC DNA was isolated as detailed in Bielenberg et al. (2004).

BAC End Sequencing: Cesium chloride purified BAC DNA was used as a template for end sequencing of PpN018G07 and PpN089G02. Sequencing reactions were performed using Big Dye™ v2.0 terminator chemistry (Applied Biosystems, Inc., Foster City, Calif., USA) according to the manufacturer's instructions for large insert DNA. Sequencing reactions were analyzed on an ABI PRISM 377 sequencer.

Probe Amplification and Labeling: Forward and reverse PCR primers for each of the amplifications were designed from BAC end sequences using Primer3_www.cgi v 0.2 (Rozen and Skaletsky 2000) and obtained from Integrated DNA Technologies (Coralville, Iowa, USA). The BAC from which primer pairs were designed was used as template to amplify probes. The amplification products were excised from the gel and purified using the QIAquick® Gel Extraction Kit (Qiagen, Inc., Valencia, Calif., USA).

DNA probes were labeled with α³²P-dCTP (Perkin-Elmer Life Sciences, Inc., Boston, Mass., USA) following denaturing and incubation with random DNA hexamers and the Klenow fragment of DNA polymerase (Promega Corp., Madison, Wis., USA) at 37° C. for 3 h (Sambrook and Russell 2002).

Southern Analysis: Genomic or BAC DNA digestion, separation, and Southern hybridization was performed according to standard protocols. Full details are provided in Bielenberg et al. (2004).

cDNA Isolation: Total RNA was extracted by the procedure outlined in Gevaudant et al. (1999). Total RNA samples were reverse transcribed using a poly(T)₁₇ oligomer as a primer with Invitrogen's SuperScript III First Strand Synthesis System for RT-PCR. Subsequent amplification of gene specific products was accomplished following the method of van der Linden et al. (2002), which is designed for use with low abundance transcripts and which we have employed to successfully isolate the EVG candidate cDNAs. In brief, this method entails an initial round of linear amplification with a forward primer and subsequent logarithmic amplification with a second nested forward primer and a downstream reverse primer (van der Linden et al., 2002).

Northern Analysis: Northern Hybridization was performed using RNA isolated as above and size fractionated on a 1.2% agarose formaldehyde gel using 1×MOPS as a running buffer. After separation was complete, the gel was rinsed in DI H₂O to remove the formaldehyde and the RNA was then transferred to Hybond-XL membrane by capillary blot using 10×SSC as a transfer buffer. Following transfer, the RNA was fixed to the membrane by baking at 80° C. for 2 h. The northern membrane was hybridized and washed in conditions similar to those used for the Southern hybridizations above, with the exception that the hybridization buffer consisted of 5×Denhardt's solution, 5×SSC, and 0.5% SDS. Following hybridization and washing the membrane was then exposed to autoradiographic film (Kodak X-Omat Blue XB-1, Perkin-Elmer Life Sciences, Inc., Boston, Mass., USA) for 60 d as determined by signal strength.

Lambda phage library construction and screening: Genomic DNA from a mutant tree was prepared as detailed above. Genomic DNA (25 ug) was partially restricted by incubation with 7.5 U or 5.0 U of Sau3AI (Promega Corp., Madison, Wis., USA) for 15 min at 37° C., resulting in DNA fragments ranging from 9 to 23 kb in size. Restriction reactions were stopped by adding 0.5 M EDTA (pH 8.0). The two partial restriction reactions were combined (50 μg DNA) and purified by phenol:chloroform extraction. The DNA fragments were ethanol precipitated, washed in 70% ethanol, air dried and resuspended in TE. A genomic library of the evg mutant was created using the Lambda FIX II/XhoI Partial Fill-In Vector Kit (Stratagene, La Jolla, Calif., USA) following the manufacturer's instructions. In short, sticky ends of the partially Sau3AI restricted genomic DNA were partially filled in with dGTP and dATP followed by ligation to Lambda FIX II DNA which had been predigested with XhoI. The resulting Lambda clones were packaged with the Gigapack III XL Packaging Extract (Stratagene, La Jolla, Calif., USA) and coincubated with the bacterial host strain XL1-Blue MRA(P2) to establish the titer and subsequently amplify the genomic library. Plaque lifts were made onto nitrocellulose membranes and prepared for hybridization following the manufacturer's instructions. Radiolabeled probes were generated as above and the membranes were hybridized using the same conditions as used for Southern blots, described above.

ii. Results

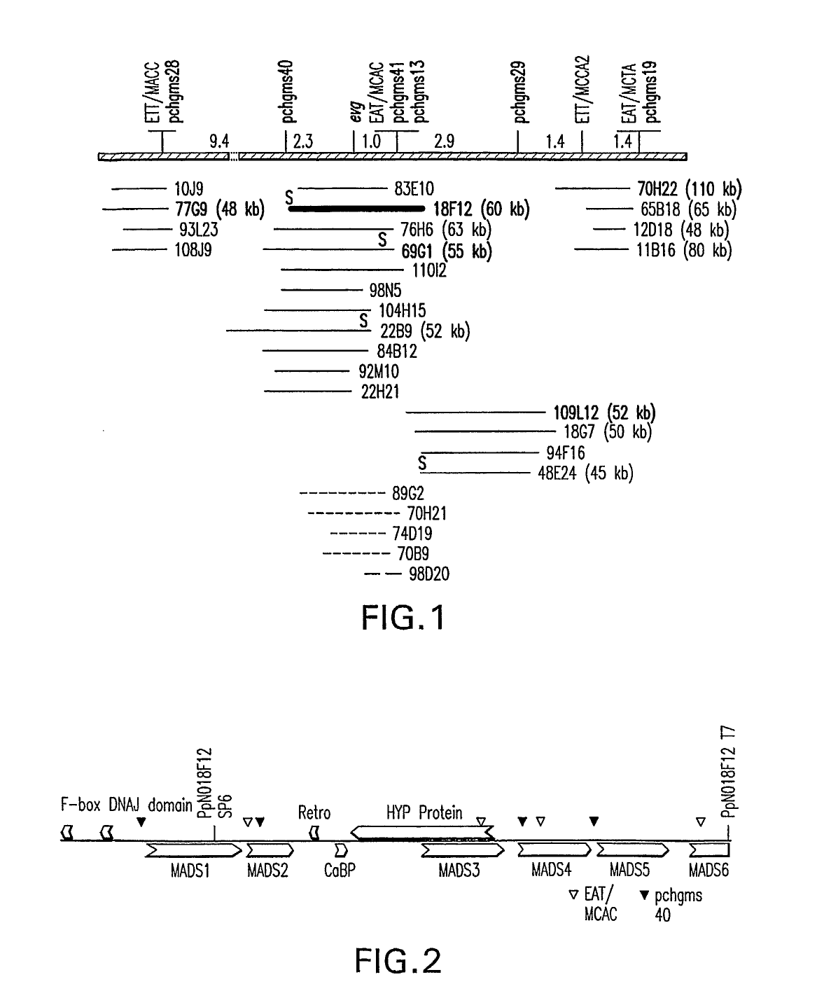

Fine mapping the evg locus: A physical map of the evergreen region was initiated from the closest STS markers. The mapping analysis of SSR markers developed from three contigs confirmed the genetic map positions of these contigs. a chromosomal walk was initiated in both directions from the BAC PpN18F12 (Prunus persica ‘NemaRed’ 18F12) containing the EAT/MCAC marker (FIG. 1). SSR marker pchgms29 was developed from PpN109L12 and mapped between EAT/MCAC and ETT/MCCA2, with 2.9 cM from EAT/MCAC. SSR markers, pchgms40 and pchgms41, were developed from PpN069G01. Pchgms41 cosegregates with the EAT/MCAC AFLP marker, while pchgms40 mapped between evergreen locus and ETT/MACC. Subsequent preliminary sequencing showed PpN069G01 to be completely internal to PpN018F12. Therefore, the evergreen locus was understood to be covered by the BAC clone PpN18F12 (FIG. 1).

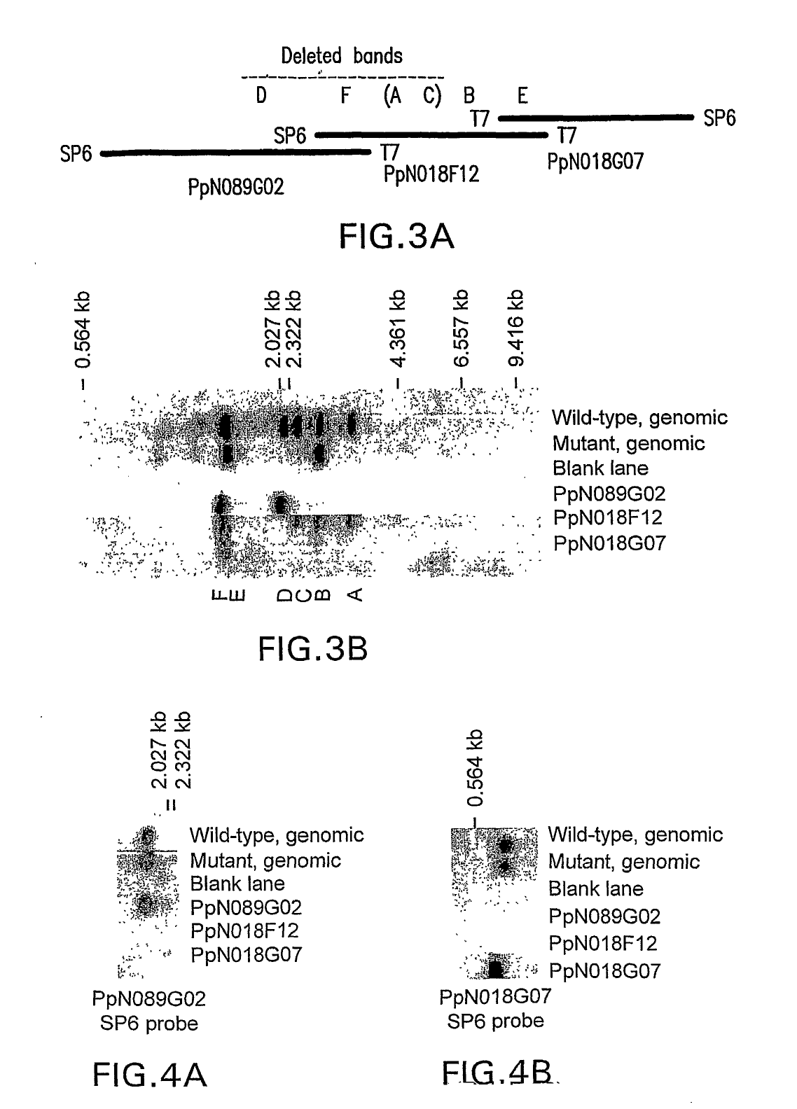

Subsequent sequencing and Southern analysis (see below) revealed the presence of what appeared to be a large tandem duplication of genes and markers in the peach genome at the EVG locus (FIG. 2, Table 3, FIG. 3). Therefore additional BACs (PpN089G02 and PpN018G07) flanking PpN018F12 were included in the sequencing effort to assure that all possible duplicated marker sites were identified.

Sequencing of the EVG locus: sequencing was completed for a 70.5 kb region (BAC PpN018F12 and partial sequencing of PpN089G02) of the peach genome putatively containing the EVG locus. Analysis of the sequences in the sequenced locus predict several candidates for the evg mutation (Table 3, FIG. 2). Analysis of the sequences information from all three BACs confirms that all of the flanking markers used to fine map the position of the locus were identified and therefore the extent of the candidate genes in the region should have been delimited. The region sequenced appears to contain at least six highly similar copies of the MIKC structural class of MADS-box genes as inferred from the presence of multiple MADS-box, K-box, and conserved C-terminal motifs (Johansen et al., 2002). This gene copy number has been confirmed by Southern hybridization of a probe for the MADS-box ORF against restriction enzyme digested peach genomic DNA as well as the BACs comprising the genomic region (FIG. 3). These MADS-box genes are highly similar to AGAMOUS-LIKE 24 from Arabidopsis. Recent evidence has shown that AGAMOUS-LIKE 24 mediates the integration of photoperiod, vernalization and GA-responsive pathways that regulate the transition from vegetative to floral meristem identity in Arabidopsis (Yu et al., 2002). LeJOINTLESS is a MADS-box transcription factor controlling the development of the abscission zone in tomato fruit pedicels. Due to their homologies with genes known to be associated with control of vegetative tissue development, the MADS-box genes found in the EVG locus present strong candidates for the evergreen gene (Mao et al., 2001; Johansen et al., 2002).

In addition to the MADS-box sequences (FIG. 2, Table 3), the sequenced EVG region contains one other strong candidate for the mutant phenotype, a Calcium-binding protein (CaBP). The fragment hybridizing to a probe of the CaBP is missing in the evg mutant genome when compared to the wild-type genome, lending support to the suggestion that a large deletion spans this region. This gene consists of two predicted ORFs, one of which encodes an EF-hand Calcium binding domain similar to those found in calmodulin-like genes. The second predicted ORF encodes a novel amino acid sequence that contains multiple poly-valine stretches, which may indicate a hydrophobic localization, possibly to a membrane. Since signal transduction cascades in response to environmental stimuli often involve transient calcium signaling, this predicted gene is a strong candidate gene for the evg mutation.

The region affected by the apparent deletion also contains two other gene sequences. One is a copia-like retrotransposon which is would not be predicted to play a functional role in the dormancy behavior of perennial trees and as such this sequence was not considering to be a candidate for the mutation (FIG. 2, Table 3). A large, hypothetical protein is also predicted in the region (FIG. 2, Table 3).

Structural analysis of the evg mutation: Analysis of Southern hybridizations of the EVG region using probes of a MADS-box ORF found in the EVG locus show the complete absence of fragments in the evg mutant DNA samples compared to the DNA of a wild-type sibling (FIG. 3; Bielenberg et al., 2004). This indicates an extensive rearrangement or deletion in this region affecting four to five of the candidate genes present in the sequenced region. The EVG gene region contained six potential MADS-box transcription factor sequences, and the deletion in EVG affected at least four of these.

TABLE 3
Gene prediction in the EVG region.

| ID | Designation | Strand | Region | Exons | Predicted gene product |
|---|---|---|---|---|---|
| EVG.1 | Putative protein | + | 186-1079 | 1 | Sim. to auxin-induced protein in |
| | | | | | Arabidopsis |
| EVG.2 | Putative carbonic | − | 14934-12558 | 7 | Similar to Arabidopsis carbonic |
| | anyhydrase | | | | anhydrase |
| EVG.3 | DNAJ-like | − | 44759-43777 | 1 | |
| EVG.4 | MADS1 | + | 49584-56338 | 7 | MIKC type MADS-box transcription |
| | | | | | factor |
| EVG.5 | MADS2 | + | 58627-63224 | 7 | MIKC type MADS-box transcription |
| | | | | | factor |
| EVG.6 | Retro | − | 65515-64934 | 1 | Copia-like retrotransposon with LTR |
| EVG.7 | CaBP | + | 67735-68878 | 2 | Ca²⁺-binding protein |
| EVG.8 | MADS3 | + | 76444-84735 | 7 | MIKC type MADS-box transcription |
| | | | | | factor |
| EVG.9 | MADS4 | + | 86773-94258 | 7 | MIKC type MADS-box transcription |
| | | | | | factor |
| EVG.10 | MADS5 | + | 95960-103242 | 7 | MIKC type MADS-box transcription |
| | | | | | factor |
| EVG.11 | MADS6 | + | 105539-111492 | 7 | MIKC type MADS-box transcription |
| | | | | | factor |
| EVG.12 | PsRT17-1-like | + | 113332-116939 | 10 | Similar to pea protein PsRT17-1 |
| EVG.13 | Kinesin-like | + | 131386-131496 | 1 | Sim. to chromosome associated |
| | | | | | kinesin |

Region values are inclusive of all exons in protein. Only predicted proteins with significant similarities to accession numbers in the NCBI and SwissProt database are listed.

A number of structural rearrangements could be responsible for the absence of hybridization bands in the mutant (FIG. 3, Bielenberg et al., 2004). These sequences can be a series of tandem duplicated loci, of which certain pairs may be independently lost in the mutant trees. Such a series of closely linked small deletions would still potentially result in the simple 3:1 Mendellian inheritance of the Evergrowing trait (Rodriguez et al. 1994) seen in the F₂ mapping population. However, the simplest explanation would be presence of a single large deletion in the mutant that results in the four missing bands (FIG. 3).

Polymorphism between the wild-type and mutant DNA for a probe, which falls within the BAC contig (the MADS-box probe), showed that the BAC contig covered the region affected by the mutation (FIG. 3). The lack of polymorphism observed between wild-type and mutant DNA for probes covering the ends of the BAC contig (FIG. 4, Bielenberg et al., 2004) indicated that these probes hybridized in a region not affected by the mutation and that there was at least one HindIII restriction site unaffected by the mutation between the probes used and the deleted MADS-box fragments. Therefore, the mutation (i.e., deletion) appeared to be contained wholly within the region spanned by the three overlapping BAC clones.

Candidate Gene Expression Analysis: cDNA corresponding to the 3′UTR and 5′UTR of each of the six MADS box genes found in the EVG region were cloned from terminal shoot tissue of a wild-type dormant F₂ sibling that was shown to be homozygous dominant for the EAT/MCAC STS marker (Wang et al., 2002). In addition cDNAs was isolating from ‘Nemared’ peach (the variety used in the creation of the BAC library) at July and October time points. Therefore the predicted genes MADS1, MADS2, MADS3, MADS4, MADS5, and MADS6 from the EVG region are all transcribed in wild-type shoot tip tissues (See Table 3).

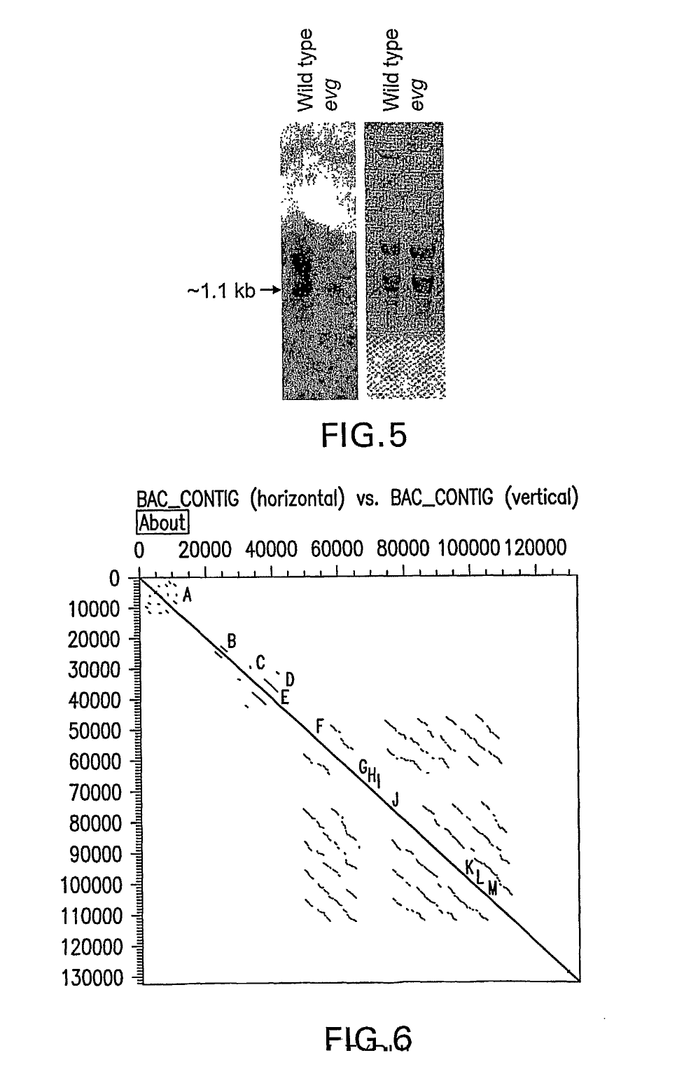

The presence of two similar copies of this gene in the genome has been confirmed by Southern analysis and can indicate a past duplication event. Northern analysis (FIG. 5) has confirmed the fact that the CaBP gene found in the EVG region is expressed in June terminal shoot tissues and that the expression is markedly reduced in the evg mutant. The residual expression that appears to remain in the mutant can be the result of cross hybridization to the alternate gene which is highly similar to the gene we have found in the EVG locus. The calculated transcript size corresponds well to the predicted 850 bp coding region with the addition of a 250 bp 3′UTR which is found on the similar, non-EVG region gene for which cDNA was successfully isolated by 3′RACE methods.

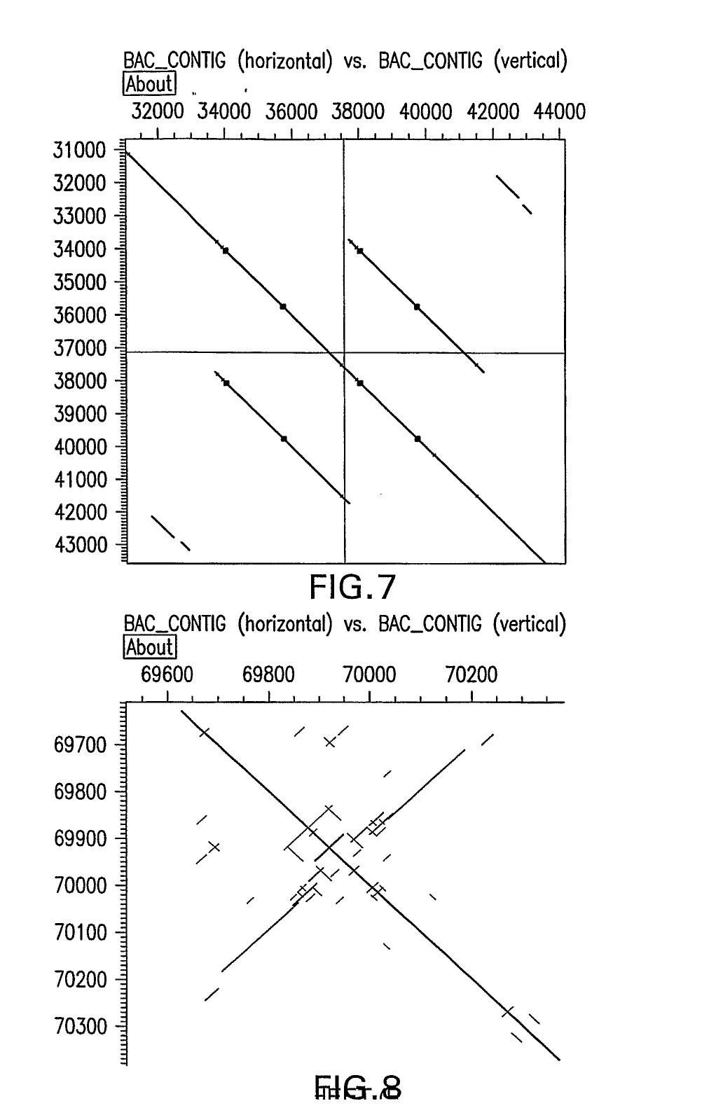

The EVG region contains multiple tandem and inverted repeats: The program ‘Dotter’ was used to perform dot-matrix analysis of the sequenced EVG region (Sonnhammer and Durbin, 1995). Dot matrix analyses of the sequenced EVG region showed multiple repeats within the region (FIG. 6). The high degree of similarity between the tandemly repeated six MIKE-type MADS box genes was evident from the dot plot (FIG. 6). Numerous other repeat regions were also located in the sequenced region. Two large tandem repeats were found at approximately 25,000 bp and 34,000 bp in the sequence (FIG. 6), the largest of which was a 4000 bp direct repeat beginning at approximately 34,000 bp and ending at approximately 42,000 by (FIG. 7). Several inverted repeat structures were also detected in the region, the largest of which was an approximately 200 bp inverted repeat centered around 70,000 bp (FIG. 8).

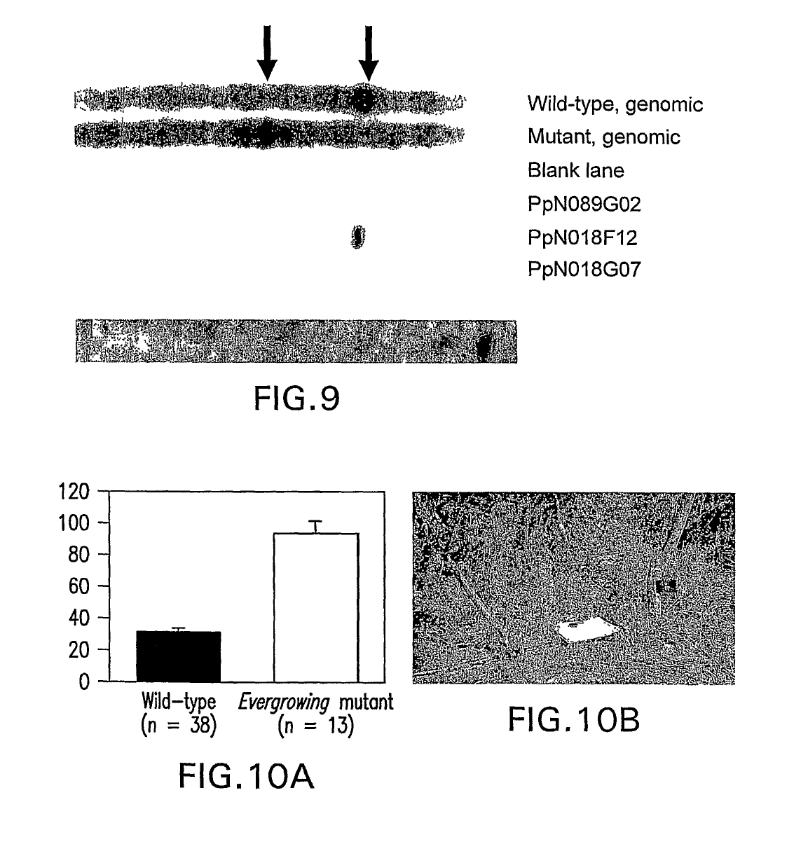

Cloning of mutant genomic locus: Previous experiments suggest that the evg mutant phenotype results from a large deletion present in the mutant genome that affects up to six candidate genes (Bielenberg et al., 2004). Southern analysis was subsequently used to walk from each end of the sequenced EVG region with probes from the wild-type region hybridized against digested genomic DNA from the wild-type and mutant trees. The hybridizations showed that the ends of the sequenced contig are not affected by a deletion. Probes from the middle of the sequenced contig fail to hybridize with the mutant genome suggesting a deletion of approximately 40-45 kilobases. Probe 18HB09 from the hybridization walking experiments resulted in a restriction fragment size polymorphism between the wild-type and mutant genomic DNA (FIG. 9). This polymorphism was interpreted as indication that 18HB09 was located on the border of the deletion in the mutant genome. This DNA probe was then used to screen a Lambda phage library created from the evg mutant genome. All positive clones identified from the lambda library were isolated and rescreened three times, resulting in six positive clones that hybridized to the 18HB09 probe. These lambda clones were fingerprinted by restriction fragment digestion and appear to have significant overlap.

### 2. Example 2

### Modifying Winter Dormancy Traits Increases Tree Biomass

A major life history trait of trees is the ability to become physiologically dormant in order to avoid unfavorable climatic conditions. Mutants that fail to cease growth and enter dormancy under dormancy-inducing conditions have been described in only two tree species, Corylus avellana L. (Thompson et al. 1985) and Prunus persica (L.) Batsch (Rodriguez et al. 1994). The best described mutant of the two is the Evergrowing peach [P. persica (L.) Batsch] mutant, a non-dormant genotype identified from southern Mexico (Rodriguez et al. 1994; Werner and Okie 1998). Evergrowing peach maintains continuous apical growth and has persistent leaves even when exposed to shortened days and low temperatures (Rodriguez et al. 1994). Although the frost hardiness of the trees is reduced as a result of impaired dormancy (Arora and Wisniewski 1996; Arora et al. 1996; Arora et al. 1992; Rodriguez et al. 1994), in relatively mild winter climates such as the southeastern United States, the many weeks of extended growth period available to this mutant relative to wild-type peaches leads to dramatic increases in tree size. After only two years of growth in the field, Evergrowing mutant trees have three times the cross-sectional trunk area of F2 sibling wild-type trees (FIG. 9). Thus, the increased growing season resulting from inhibited winter dormancy leads to increased tree biomass.

### 3. Example 3

### Generation of Transgenic ‘Knock-Outs’ in Poplar

Poplar was chosen for use as a transgenic system in which to investigate EVG candidate gene function. Two approaches can be taken. First, the peach EVG candidates cDNAs (the MADS box genes and the CaBP) can be used in the creation of chimeric constructs for RNAi and overexpression studies to attempt to phenocopy the evg mutation in poplar. Two such constructs are completed, and poplar have been transformed with these constructs in the calli stage. Second, the genes from poplar that have a high sequence similarity to the EVG candidate genes from peach can be identified and used in similar RNAi and overexpression experiments to phenocopy the evg mutation with the native poplar genes. Peach cDNA sequences can be similar enough to the poplar orthologues to induce suppression. For example, the poplar orthologues can comprise a gene having a nucleic acid sequence with at least 70%, 75%, 80%, 85%, 90%, 95% identity to the sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

A set of eight MADS-box genes have been identified from the preliminary poplar sequence assembly that have considerable similarity (40-70% amino acid identity) to the MADS-box genes in the EVG region. In addition to the relatively highly conserved MADS and K-box domains shared by many MADS box sequences, the peach and poplar genes also share a conserved 3′ amino acid motif (SLKLGL, SEQ ID NO:9) immediately preceding the stop codon that is conserved among vegetatively expressed MADS-box genes (Alvarez-Buylla et al., 2000). Interestingly four of these poplar MADS box genes are found on one poplar sequence contig of approximately 330 kb, which indicates that they can have a similar clustered arrangement as the genes in the EVG locus. poplar genomic sequences can be searched to identify sequences that are similar to the peach CaBP candidate gene.

i. Methods

Isolation and cloning of complete cDNAs for each peach candidate gene: A gene has already obtained specific 5′UTR or 3′UTR containing cDNA for the MADS genes in the EVG locus that are affected by the large deletion we have detected in the mutant genome between flanking genetic markers.

Eight poplar MADS box genes of high similarity to the MADS-box containing genes in the EVG locus of the peach genome have identified by blast algorithms. Amplifications of gene specific regions of the poplar genes can be carried out on genomic poplar DNA or by RT-PCR of poplar mRNA isolated from tissues shown to express the genes in peach. RT-PCR experiments can be carry out to obtain complete sequences of the poplar putative orthologes prior to transgenic phenocopy experimentation.

Vector Construction: For construction of chimeric genes for poplar transformation the Agrobacterium binary vectors pB7GWIWG2II and pBtWG2 can be used. These are GATEWAY™ T-DNA binary vector that facilitate the construction of both RNAi and overexpression genes (Karimi et al. 2002; http://www.plantgenetics.rug.ac.be/gateway/pB7GWIWG2map.html). For RNAi vectors the appropriate cDNA can be cloned into the pENTR D-TOPO entry vector followed by recombination into the GATEWAY™ T-DNA binary vector pB7GWIWG2II using LR Clonase™ enzyme mix (Invitrogen). The resulting binary vector can contain an expression cassette consisting of the CaMV 35S promoter::cDNA sense—Arabidopsis intron—cDNAantisense::35S terminator and the plant selectable marker bar. For the overexpression of chimeric genes, the appropriate gene or full length cDNA can be cloned into the pENTR D-TOPO entry vector followed by recombination into the GATEWAY™ T-DNA binary vector pB7WG2 as described for the RNAi vector. The resulting binary vector can contain the expression cassette CaMV 35S promoter::cDNA or gene::35S terminator and the plant selectable marker bar.

Plant Transformation: Transgenic poplar can be generated using the hybrid aspen clone 717-1B4. Genetic transformation can be conducted as described by Leple et al. (1992). Transformed shoots can be selected based upon resistance to glufosinate ammonium (5 mg/l). Regenerated shoots resistant to glufosinate ammonium can be rooted in vitro, and the transgene insertion can be verified by DNA gel blot analysis. Transformed plants can be in vitro propagated for experimental use. At least 5 independent RNAi lines can be regenerated and multiplied for each cDNA.

Phenotypic evaluation: All transgenic lines can be vegetatively propagated to provide sufficient plants for replicated studies. The morphology and physiology of apical buds of transgenic trees can be compared with non-transformed regenerants and empty-vector transformants during the induction of bud dormancy of plants exposed to natural conditions and plants grown in controlled environment chambers. To compare bud morphology, representative apical buds can be collected at regular time intervals from the various plants grown in natural or controlled environments, immediately fixed in formalin-acetic acid-alcohol (FAA), dehydrated in an ethanol series and paraffin embedded. 10-15 μm thin longitudinal and cross sections can be made using a microtome and stained with safranin O and fast green. Sections can be examined microscopically for differences in bud morphology and anatomy and photographed. The number and size of the various bud organs (bud scales, stipules and embryonic leaves) can be made and recorded.

Comparisons of bud physiology can concentrate on two traits, growth cessation and bud dormancy. Growth cessation can be evaluated in the various transgenic lines and controls in both natural and controlled environment conditions by making regular measurements of stem growth during the induction of bud dormancy. To measure dormancy status, plants of the various transgenic lines and controls exposed to natural conditions or controlled environments can be defoliated at regular intervals during dormancy initiation and returned to growth permissive conditions (long-days and warm temperatures, i.e 25° C.). Plants are then observed daily and the days recorded until at least one leaf has emerged from the bud. For both dormancy measurements and growth cessation, differences can be determined by ANOVA using the GLM procedure of SAS with each bud or stem serving as a replicate.

### 4. Example 4

### Use a Transgenic Tobacco System for the Rapid Assay of Peach EVG Candidate Gene Regulation in Response to Environmental and Hormonal Cues

Molecular and functional genomics approaches are increasingly being adopted for economically or ecologically important species that do not have developed transformation and regeneration methodologies, such as is the case for peach. Once genes are cloned and identified in these species, further functional testing of promoter elements and the use of reporter gene constructs for localization at the cellular and tissue level are sometimes difficult in the species from which the genes were isolated. A common strategy to overcome this limitation is to make use of an easily transformed model species such as tobacco, arabidopsis (Arabidopsis thaliana), or tomato (Lycopersicon esculentum) for detailed analysis of tissue or cell-type localization of gene expression, regulation of expression by environmental or biochemical stimuli, and the analysis of cis-regulatory elements located in the promoter region (Moon and Callahan, 2004; Avila et al., 2002; Ishizaka et al., 2003; Ko et al., 2003).

In parallel with the transgenic analysis of the poplar putative orthologues of the EVG candidate genes, a heterologous transgenic expression approach can be used to analyze the tissue localization and environmental and biochemical regulation of the EVG candidate genes in tobacco (Nicotiana tabacum). the expression patterns and environmental responsiveness of the promoters of the EVG candidates can be analyzed as an initial step to place these genes in a framework of environmental and biochemical signaling that must take place for shoot tissues to arrest growth, develop buds and enter dormancy in response to dormancy inducing conditions. Because the candidate genes have been identified by a forward genetics approach, it is not yet clear how these genes are receiving information from the environmental perception that must be taking place during dormancy induction. The use of a transgenic tobacco system with peach promoter-reporter gene constructs can be an efficient strategy to screen the set of candidate genes under a variety of conditions.

i. Methods

Promoter region isolation and cloning: 5′ upstream regions of the six MADS box containing genes and the CaBP gene can be amplified from the BACs PpN018F12 and PpN089G02 by PCR. PCR primers for each of the promoter regions can be designed with additional adaptors at the 5′ ends of the oligonucleotides containing six-base restriction enzyme cut sites to allow for directional cloning of the amplified promoter into the binary plasmid to be used for transformation. An approximately 1500 bp region upstream of the transcription start site for each gene can be used for initial promoter activity analysis. This length corresponds to the approximate sequence length between the experimentally determined transcription start site of the MADS-box genes MADS2 through MADS6 and the end of the 3′UTR region of the gene immediately upstream of the respective gene.

Vector construction: An Agrobacterium binary vector containing the Nospro-nptII-Noster and the 35Spro-sGFP-35Ster cassettes can be used to construct the chimeric genes for promoter analysis. Promoter regions cloned by PCR using ligation adaptors can be restricted, isolated and ligated into the binary vector in position to replace the 35Spro region in the GFP cassette resulting in a EVG candidate promoter::sGFP::35Ster chimeric construct. One construct can be created for each of the 6 MADS-box containing genes within the EVG locus as well as the CaBP gene.

Plant transformation: Transgenic tobacco can be generated using N. tabacum cv. Xanthi using the leaf disc transformation method described in Horsch et al. (1985). Transformed shoots can be selected based upon resistance to kanamycin. Regenerated shoots resistant to kanamycin will be rooted in vitro, and the transgene insertion can be verified by DNA gel blot analysis. Transformed plants can be in vitro propagated for experimental use. At least five independent lines can be regenerated and propagated for each promoter-GFP fusion construct.

Reporter gene evaluation: Reporter gene activity can be monitored by examination of tissues using a fluorescence stereomicroscope while exciting with blue light. GFP excitation fluorescence is easily distinguished from the background chlorophyll fluorescence in green tissues and eliminates the requirement for substrate incubation and tissue clearing necessary in reporter systems such as GUS. Photographic documentation can be used for data acquisition and comparison of expression data between promoters and environmental conditions.

GFP fluorescence can be observed in all transgenic lines for evaluation of tissue localization. Propagated replicate plants can be to assay for promotion or inhibition of fluorescence under a wide variety of environmental stimuli and biochemical activity that are associated dormancy inducing conditions. Specifically these can include: transfer from long day to short day conditions, low temperatures, combinations of altered photoperiod and low temperature exposure, progressive drought, and exogenous treatment of plant tissues with the plant hormones ABA, GA, auxins, and ethylene. While visual intensity can provide some information for responsiveness of the promoter to inducing conditions, quantitative PCR approaches can also be used to determine relative expression levels. Potential differential response patterns of the promoters can be scored and correlated with the presence or absence of conserved cis-element motifs in the promoter sequence. When an EVG candidate gene is found to be specifically regulated by particular environmental or biochemical exposure, promoter deletion experiments can will be used to correlate specific regions in the promoter sequence with responsiveness to stimuli.

### F. REFERENCES

Alvarez-Buylla E. R., Liljegren S. J., Pelaz S., Gold S. E., Burgeff C., Ditta G. S., Vergara-Silva F. & Yanofsky M. F. (2000) MADS-box gene evolution beyond flowers: expression in pollen, endosperm, guard cells, roots and trichomes. Plant Journal, 24, 457-466.
Arora R, Wisniewski M (1996) Accumulation of a 60-kD dehydrin protein in peach xylem tissues and its relationship to cold acclimation. Hortscience 31:923-925
Arora R, Wisniewski M, Rowland L J (1996) Cold acclimation and alterations in dehydrin-like and bark storage proteins in the leaves of sibling deciduous and evergreen peach. Journal of the American Society for Horticultural Science 121:915-919
Arora R, Wisniewski M E, Scorza R (1992) Cold-acclimation in genetically related (sibling) deciduous and Evergreen peach (Prunus persica L Batsch). 1. Seasonal changes in cold hardiness and polypeptides of bark and xylem tissues. Plant Physiology 99:1562-1568
Aswath C. R. & Kim S. H. (2005) Another story of MADS-Box genes—their potential in plant biotechnology. Plant Growth Regulation, 46, 177-188.
Aswath C. R., Mo S. Y., Kim S. H. & Kim D. H. (2004) IbMADS4 regulates the vegetative shoot development in transgenic chrysanthemum (Dendrathema grandiflora (Ramat.) Kitamura). Plant Science, 166, 847-854.
Bielenberg D G, Wang Y, Fan S, Reighard G L, Scorza R, and Abbott A G, 2004. A deletion affecting several gene candidates is present in the Evergrowing peach mutant. J Hered 95:436-444.
Brill E. M. & Watson J. M. (2004) Ectopic expression of a Eucalyptus grandis SVP orthologue alters the flowering time of Arabidopsis thaliana. Functional Plant Biology, 31, 217-224.
Broothaerts W., Keulemans J. & Van Nerum I. (2004) Self-fertile apple resulting from S-RNase gene silencing. Plant Cell Reports, 22, 497-501.
Chao W. S. (2002) Contemporary methods to investigate seed and bud dormancy. Weed Science, 50, 215-226.
Doyle J J and Doyle J L, 1990. Isolation of plant DNA from fresh tissue. Focus 12:13-15.
Garcia-Maroto F., Carmona M. J., Garrido J. A., Vilches-Ferron M., Rodriguez-Ruiz J. & Alonso D. L. (2003) New roles for MADS-box genes in higher plants. Biologia Plantarum, 46, 321-330.
Georgi L L, Wang Y, Yvergniaux D, Ormsbee T, Inigo M, Reighard G, and Abbott A G, 2002. Construction of a BAC library and its application to the identification of simple sequence repeats in peach Prunus persica (L.) Batsch. Theoretical and Applied Genetics 105:1151-1158.
Hily J. M., Scorza R., Malinowski T., Zawadzka B. & Ravelonandro M. (2004) Stability of gene silencing-based resistance to Plum pox virus in transgenic plum (Prunus domestica L.) under field conditions. Transgenic Research, 13, 427-436.
Johansen B., Pedersen L. B., Skipper M. & Frederiksen S. (2002) MADS-box gene evolution-structure and transcription patterns. Molecular Phylogenetics and Evolution, 23, 458-480.
Kim S. H., Mizuno K. & Fujimura T. (2002) Isolation of MADS-box genes from sweet potato (Ipomoea batatas (L.) lam.) expressed specifically in vegetative tissues. Plant and Cell Physiology, 43, 314-322.
Lohmann J. U. & Weigel D. (2002) Building beauty: The genetic control of floral patterning. Developmental Cell, 2, 135-142.
Mao L, Begum D, Chuang H W, Budiman M A, Szymkowiak E J, Irish E E, and Wing R A, 2000. JOINTLESS is a MADS-box gene controlling tomato flower abscission zone development. Nature 406:910-913.
Mlotshwa S., Voinnet O., Mette M. F., Matzke M., Vaucheret H., Ding S. W., Pruss G. & Vance V. B. (2002) RNA silencing and the mobile silencing signal. Plant Cell, 14, S289-S301.
Parenicova L., de Folter S., Kieffer M., Horner D. S., Favalli C., Busscher J., Cook H. E., Ingram R. M., Kater M. M., Davies B., Angenent G. C. & Colombo L. (2003) Molecular and phylogenetic analyses of the complete MADS-box transcription factor family in Arabidopsis: New openings to the MADS world. Plant Cell, 15, 1538-1551.
Prakash A. P. & Kumar P. P. (2002) PkMADS1 is a novel MADS box gene regulating adventitious shoot induction and vegetative shoot development in Paulownia kawakamii. Plant Journal, 29, 141-151.
Ratcliffe O. J., Kumimoto R. W., Wong B. J. & Riechmann J. L. (2003) Analysis of the Arabidopsis MADS AFFECTING FLOWERING gene family: MAF2 prevents vernalization by short periods of cold. Plant Cell, 15, 1159-1169.
Rodriguez J, Sherman W B, Scorza R, Wisniewski M, Okie W R (1994) Evergreen peach, its inheritance and dormant behavior. Journal of the American Society for Horticultural Science 119:789-792
Rosin F. M., Hart J. K., Van Onckelen H. & Hannapel D. J. (2003) Suppression of a vegetative MADS box gene of potato activates axillary meristem development. Plant Physiology, 131, 1613-1622.
Rozen S and Skaletsky H J, 2000. Primer3 on the WWW for general users and for biologist programmers. In: Bioinformatics Methods and Protocols: Methods in Molecular Biology, (Misener S, eds). Totowa, N.J.: Humana Press; 365-386.
Sambrook J and Russell D W, 2002. Molecular Cloning: A laboratory manual. Cold Spring Harbor, N.Y.: Cold Spring Harbor Laboratory Press.
Thompson M M, Smith D C, Burgess J E (1985) Non-dormant mutants in a temperate tree species, Corylus avellana L. Theoretical and Applied Genetics 70:687-692
van der Linden C. G., Vosman B. & Smulders M. J. M. (2002) Cloning and characterization of four apple MADS box genes isolated from vegetative tissue. Journal of Experimental Botany, 53, 1025-1036.
Vergara-Silva F., Martinez-Castilla L. & Alvarez-Buylla E. R. (2000) MADS-box genes: Development and evolution of plant body plans. Journal of Phycology, 36, 803-812.
Wang Y, Garay L, Georgi L L, Reighard G L, Scorza R, and Abbott A G, 2002a. Development of Bacterial Artificial Chromosome contigs in the Evergrowing gene region in peach [Prunus persica (L.) Batsch]. Acta Horticulturae 592:183-189.
Wang Y, Georgi L L, Reighard G L, Scorza R, and Abbott A G, 2002b. Genetic mapping of the evergrowing gene in peach Prunus persica (L.) Batsch. Journal of Heredity 93:352-358.
Werner D J, Okie W R (1998) A history and description of the Prunus persica plant introduction collection. Hortscience 33:787-793
Yao J. L., Dong Y. H. & Morris B. A. M. (2001) Parthenocarpic apple fruit production conferred by transposon insertion mutations in a MADS-box transcription factor. Proceedings of the National Academy of Sciences of the United States of America, 98, 1306-1311.
Yao J. L., Dong Y. H., Kvarnheden A. & Morris B. (1999) Seven MADS-box genes in apple are expressed in different parts of the fruit. Journal of the American Society for Horticultural Science, 124, 8-13.
Yu H, Xu Y F, Tan E L, and Kumar P P, 2002. AGAMOUS-LIKE 24, a dosage-dependent mediator of the flowering signals. Proceedings of the National Academy of Sciences of the United States of America 99:16336-16341.

1. A method of modulating the growing season of a perennial tree, comprising deleting or suppressing one or more target genes in the evergreen locus.

2. The method of claims 1, wherein the perennial tree is a poplar.

3. The method of claim 1, wherein the evergreen locus is an allelic variant of the Evergrowing (EVG) peach locus.

4. The method of claim 1, wherein the growing season of a perennial tree is increased.

5. The method of claim 1, wherein the biomass of the perennial tree is increased at least about 5%, 10%, 15%, 20%, 30%, 40%, 50%, 60% per year as compared to a control tree.

6. The method of claim 1, wherein the gene is a MADS-box gene encoding a transcription factor.

7. The method of claim 6, wherein the gene comprises a sequence having at least 70%, 75%, 80%, 85%, 90%, or 95% identity to the nucleic acid sequence set forth in SEQ ID NO:1, SEQ ID NO:2, SEQ ID NO:3, SEQ ID NO:4, SEQ ID NO:5, SEQ ID NO:6, or SEQ ID NO:7.

8. The method of claim 1, wherein the gene encodes a Calcium binding protein.

9. The method of claim 8, wherein the gene comprises a sequence having at least 70%, 75%, 80%, 85%, 90%, or 95% identity to the nucleic acid sequence set forth in SEQ ID NO:8.

10. The method of claim 1, wherein the gene is deleted using homologous recombination.

11. The method of claim 1, wherein the gene is suppressed using a functional nucleic acid.

12. The method of claim 11, wherein the functional nucleic acid is an antisense, ribozyme, siRNA, or shRNA.

13. The method of claim 12, wherein the functional nucleic acid comprises a sequence having at least 70%, 75%, 80%, 85%, 90%, or 95% identity to a portion of the nucleic acid sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

14. A method of modulating winter dormancy of a perennial tree, comprising administering to the tree a nucleic acid comprising one or more target genes in the evergreen locus.

15. The method of claim 14, wherein the nucleic acid is administered to the tree in an Agrobacterium vector.

16. The method of claim 14, wherein winter dormancy is prematurely activated in the tree.

17. The method of claim 14, wherein the nucleic acid is functionally linked to an expression control sequence.

18. The method of claim 17, wherein the expression control sequence is an inducible promoter.

19. The method of claim 14, wherein the perennial tree is a poplar.

20. A nucleic acid comprising the sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

21. A nucleic acid that hybridizes under stringent conditions to the nucleic acid sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

22. A nucleic acid comprising a sequence having at least 70%, 75%, 80%, 85%, 90%, or 95% identity to the nucleic acid sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

23. A polypeptide encoded by the nucleic acid of claim 20.

24. A vector comprising the nucleic acid of claim 20.

25. The vector of claim 24, wherein the nucleic acid is functionally linked to an inducible promoter.

26. A cell comprising the vector of claim 24.

27. A plant comprising the vector of claim 24.

28. A method of making a transgenic organism comprising administering the nucleic acid of any of claim 20.

29. A method of making a transgenic organism comprising administering the vector of claim 24.

30. A method of making a transgenic organism comprising administering the cell of claim 26.


SEQUENCE LISTING (24 sequences)

SEQ ID NO: 1 - DNA, 998 bp, Artificial Sequence
  Description of Artificial Sequence/note = Synthetic Construct
ttcttgaacagccagccaccaccagcagcggcagtcaactctctctctctctcctctctctcattctcttaaattttctggcttcagaccctgaaactccccgacaaaggggacgatgaaaatgacgagggagaagatcaagatcaagaagattgacaacttgcctgcaaggcaagtgaccttctcaaagaggaggagagggatcttcaagaaagctgcagagttatctgttctgtgtgaatcggaggtggcagttgtcatcttttctgctactggcaagctttttgattattcaagctcaagtatgaaggatgttattgaaaggtaccaagagcacataaatggtgctgaaaaatttgacgagccgtctattgagttgcagccagagaaagaaaaccacatcagattgagcaaggaacttgaggagaagagccgccagctgaggcagatgaaaggagaggatcttgaagagctgaattttgatgagttgcagaagttagaacaactggtggatgcaagccttggccgggtgattgaaactaagatgctatccagaggaaatattggacctgcgcttatggagccggagaggttgaataataatattggtggtggaggagaagaagaaggcatgtcatctgaatctgctacctccaccacctgcaacagtgctcccagtctctctcttgaagatgactccgacgacgtcactttatctctcaaactggggcttccataatttctctggttaaaaagtggagggttgtgattgtgtgtaagataaataaagaaaatcgagatattgtaagatatcagtgattgtgtgtgtcttaactcgatacaagattgaaaaatattgaggggaaagtaacttgtgttgtatctaaacctatgtattttatgcaacctgacatattgctctttgacttgtattgtctttagaactctgtatcaatgcagcctgctttggagactctaaaaaaaaaaaaaaaaaaaaa

SEQ ID NO: 2 - DNA, 1059 bp, Artificial Sequence
  Description of Artificial Sequence/note = Synthetic Construct
gcctttgaattattctctttcttgaacagccagccaccaccagcagcggcagtcaactctctctctctctcctctctctcattctcttaaattttctggcttcagaccctgaaactccccgacaaaggggacgatgaaaatgatgagggagaagatcaagatcaagaagattgacaacttgcctgcaaggcaagcgaccttctcaaagaggaggagagggatcttcaagaaagctgcagagttatctgttctgtgtgaatcggaggtggcagttgtcatcttttctgctactggcaagctttttgattattcaagctcaagtatgaaggatgttattgaaaggtaccaagagcacataaatggtgctgaaaaatttgacgagccgtctattgagttgcagccagagaaagaaaaccacatcagattgagcaaggaactggaggagaagagccgccagctgaggcagataaaaggggaggatcttgaagagctgaattttgatgagttgcagaagttagaacaactggtggacgcaagccttggccgtgtgattgaaactgagggagctgagctggtagaagccaacaaccagctacggcagaggatggtgatgttatccagaggaaatattggacctgcgcctacggagccggagaggttcgttaataatattggaggtggaggagaagaaggcatgtcatctgaatctgccacaaatgcaaccatcagcagctgcagcagtggtcccagtctctctcttgaagatgactgctccgacgtcactttagctctcaaactggggcttccctaatttctatggttaagaaatggtgaaagatgaaagtttactctccttatagctaatcttaatttctatggttaagaagtgaagaaagatgaagatttactctccttatagctaaataaaattaatgtattgtaagatattagtgattatatgtcttaactcgatacaaaattaaaaaatattgaagggagagtaactttgtgttgtatctaaacctatctattacatgcagcctaacatatttctctttg

SEQ ID NO: 3 - DNA, 1096 bp, Artificial Sequence
  Description of Artificial Sequence/note = Synthetic Construct
acgcggggaatcccttccttcgttaaacagtcagccagcaggagaagcagccatttctctctcccattctcttagtttttctgagaacaccaaaacttcagaccctgaaaccccccaaaaaggggggatggtgaagacgaatgaggaagaagatcaagatcaagaagattgactacttgcctgcaaggcaggtgaccttctcaaagaggaggagagggatcttcaagaaagctgaggagctatctgttctgtgtgaatctgaggtggcagtagtcatcttttctgctactggcaagctttttgattattcaagctcaagtacaaaggatgttgttgaaaggtatcaagcgcacacaaatggtgtcgaaaaatcggacgaaccgtctgttgagctgcagctagaaattgaaaaccacatcagattgaccaaggaacttgaggagaagagctgccagctgaggcagataaaaggggaggatcttgaagagctgaattttgatgagttgcagaagttagaacaactggtggacgcaagccttggccgtgtgattgaaactgaggaagaactgattatgagtgagattatggcacttgaaagaaagggagctgagctggtagaagccaacaaccagctacggcagaggatggtgatgttatccagaggaaatattggacctgcgcctacggagccggagaggttcgttaataatattggaggtggaggagaagaaggcatgtcatctgaatctgccacaaatgcaaccatcagcagctgcagcagtggtcccagtctctctcttgaagatgactgctccgacgtcactttagctctcaaactggggcttccctaatttctatggttaagaaatggtgaaagatgaaagtttactctccttatagctaatcttaatttctatggttaagaagtgaagaaagatgaagatttactctccttatagctaaataaaattaatgtattgtaagatattagtgattatatgtcttaactcgatacaaaattaaaaaatattkgaagggagagtaacytttgtgtttgtatctaaacctatctattacatgccagccctaaccattt

SEQ ID NO: 4 - DNA, 1062 bp, Artificial Sequence
  Description of Artificial Sequence/note = Synthetic Construct
ctctctcattctcttagtttttctggacacaccaaaacttcagacctgaaacctcccaacgaaggggggatggtgaagatgatgaggaagaagatcaagatcaagaagattgattacttgcctgcaaggcaggtgaccttctcaaagaggagaagagggatcttcaagaaagctgcagagctatctgttctgtgtgaatctgaggtggcagttgtcatcttttctgctactgacaagctctttgattattccagctcaagtatcaaggatgttattgaaaggtacaaagcacacacaaatggtgtcgaaaaatcggacaaaccgtctgttgagctacagctagagaatgaaaaccacatcagattgagcaaggaactcaaggagaagagccaccagctgaggcagatgaaagcagaggatcttgaagagctgaattttgatgagttgcagaagttagaacaactggtggacgcaagccttggccgtgtgattgaaactaaggaagaactgagaatgagtgagattatggcacttgaaagaaagggagctgagctggtagaagccaacaaccagctaaggcagacgatgatgttatccggaggaaatactggacctacgcttatggaaccggagaggttgagtaataatattggaggtggaggagaagaagaaggcatgtcatctgaatctgctatctccaccacctgcaatagtgctctcagtctctctccctctcttggagatgactccgacgacgtcactttatctctcaaactggggctttcctaatttctctggttaagaagtggagaacgataagagtttactctccttgtagctaaataaagaaagtcgaggtattataagatatcagtgattgtgtgtgttaactcgactttgttttgtatctaaacctatgtattacgtccagcctgacaaattgcgctttgacttgtattgtatttagaactttgtatcaaaatagtctgcatggagactcttgtggattttgtattgcttcaatgtcacagcacaggataacgttacgaaaaaaaaaaaaaa

SEQ ID NO: 5 - DNA, 1080 bp, Artificial Sequence
  Description of Artificial Sequence/note = Synthetic Construct
gaacagccagccatttctctctctcgttctcctagtttttctgtacgcacaacttcagaccctgaaggccccaaaagaagggggatggtgaaaatgatgagggagaagatcaagatcaagaagattgactacctgccagcaaggcaggtgaccttctcaaagaggagaagagggatcttcaagaaagctgcagagctatctgttctgtgtgaatctgaggtggcagttgtcatcttttctgctactggcaagctttttgattattcaagctcaagtatcaaggatgttattgaaaggtacgaagtgcgcacaaatggtgtcgaaaaatcggatgaacagtctcttgagctgcagctggagaatgaaaaccacaccaaactcagtacggaacttgaggagaagaaccgccagctgaggcagatgaaaggtgaggatcttgaagagctggatctggatgagttgctgaagttggaacaactggtggaagcaacccttgtccgtgtgatggaaactaaggaagaactgattatgagtgatattgtggcacttgagaaaaagggaactgagctggtagaagccaacaatcagatggtgatgttaagggagaggatggtgatgttatccaaaagaaatactggacctgcgcttatggagccatctgaatctgctacctccaccagctgcaacagtgctctcagtctctctcttgaagatgactgttccgacgacgtcgttttatctctcaaactggggctaacggtacgcgcgggtcgtagaccgatgtgcttaaagacttagatgtctttcttcattgttttcacgttacaaagctcatttcaagatatcgtgaagtttagtcccacgaatgacattataggaaccagaatgtttaagaagaaactcatataattgtctgcgtggattaaatcaacacaagtggcctggggaaagtaacctgagcaagataaagacgaagccgtgtgtcatggtgggctcaatcaagtggcttgacagcgcatgacctgtgtcaaagtgccatcctccacacccaaaaaaaaaaaaaaaaaaaaaaaaagtact

SEQ ID NO: 6 - DNA, 1045 bp, Artificial Sequence
  Description of Artificial Sequence/note = Synthetic Construct
aacaacaagacagcagcagcagcagccatttctctctctcatttcgaacgcagaccccgaaacccaccaacgaagatgatgaggaataagatcaagatcaagaagattgactacttgcctgcaaggcaggtgaccttctcaaaaaggagaagagggctcttcaagaaagctgcagagctatctgttctgtgtgaatctgaggtggcagttgtcatcttttctgctactggcaagctttttgattattcaagctcaagtaccaaggatgttattgaaaggtacaacgcagacataaatggtgtcgaaaaattgaacaatcaagagattgagctgcagctggagaatgaaaaccacatcaaactgagtaaggaactcgaggagaagagccgccagctgaggcagatgaaaggtgaggatcttgaagggctgaatctggatgagttgttgaagttggaacaactggtggaagcaagccttggccgtgtcatggaaactaaggaagagctgattaagagtgagattatggcacttgaaagaaagggaactgagctagttgaagctaacaaccagctaaggcagacgatggtgatgttatccggaggaaatactggacctgcgcttatggatccggagaggttgaataataatattgaaggtggaggagaagaagaaggcatgtcagctgaatctgctatctcccccccctgcaacagtgctgtcagtctctctcttgaagatgactcctccgatgaggtcactttgtctctcaaactggggcgttaagaagtgaagaaagatgaaagtttactctccttataactaaataaataaagttgaggtattgtaagatatcagtaattgtgtctcttaactcgacataacattgaaaaatatcaaagggagggtgactgtgtgttgtatctaaacctatgcgttacatgcaagcttgaaattgctctgactttgtattgtatttaaaactctgtattaatgcagcttgcatggtgagctaaaaaaaaaaaaaaaaaaaaaaaaaaaaaaagta

SEQ ID NO: 7 - DNA, 1099 bp, Artificial Sequence
  Description of Artificial Sequence/note = Synthetic Construct
gagccaccaccagcagccgccctttatctctctagttctcttagtttttctggacagaccaaaacttcagaccgggctgaaaccccgacaaagggaaacggtgaaaatgatgagggagaagatcaagatcaagaagattgactacctgccagcaaggcaggtgaccttctcaaagagaagaagagggctcttcaagaaagctgcagagctatctgttctgtgtgaatctgaggtggctgtcatcatcttctctgctactgacaagctctttgattattcaagctcaagtaccgaggatgttattgaaaggtacaaagcgcacacaaatgatctcgaaaaatcgaacaaacagtttcttgagctgcaactggagaatgaaaaccacatcaaactgagtaaggaactcgaggagaagagccgccagctgaggcagatgaaaggtgaggatcttcaagggctgaatatggatgagctgctgaagttagaacaactggtggaagcaagccttggccgtgtgatagaaactaaggaagagctgattatgagtgagattatggcactggagaaaaagggagctgagctggtagaagccaacaaccagttaaggcagaagatggcgatgttatccggaggaaatactggacctgcgtttgtggagccggagacgttgattactaatgttggaggtggaggagaagaagacggcatgtcatctgaatctgccataattgccacctccaccagctgcaacagtgctcacagtctctctcttgaagatgactgctccgatgtcactttatctctcaaactggggcttccctagtttcttggttaagaagtggacaaggaagaaagtttactcactttatagctaaataaacaaaatcgagataatgtgagacgcatcagtagtgatcgtgtgccttaactcgatatgacattgaagggagagtaactttctgtgtatctaaacctatggtattacatgcagcctgacatactgctcttcgactttgtattgtatgtattaatgcagcctgcacggagactactagatgttgtgttgattgggccaaaaaaaaaaaaaaaaaaaaaaagt

SEQ ID NO: 8 - DNA, 693 bp, Artificial Sequence
  Description of Artificial Sequence/note = Synthetic Construct
atggaagctttagcggctctcttacgcatcagcctcgccgtcatgagccgaaacctcgccttcaacaccaccgagaaaggcttccccatctatgaaaacttgaatcaaattcagaaaaaaaatgggcaacgatatgagcgacaaattaatatcgtcaataaaggaggcattcacgctctcgtcatcgacaacgacggtaagattgttccttcggaattagggaacctaatgcgattgcttggagtcaactcgacccaggcccaacacaaatccatcgctgccgaggagaagctcactgcccccttcaacttccctcacttcctcgaccttatgggaaagcacatgaagcccacgcccttcgatcatcagctctgcaacaccttcaaagtcctcgacaaggactccaccggcttcgtctttgtctcggagcttcgacacatactcactagcatcaacaagaaattagagccatccgagttcgcaagtggatctgggaggtcaatgtcgggtccagtgccaagtgagaagctgaacggtgtgttttcactggttaaggcccagtttgggattgctatcacttttttaaaaagttacttatgttgtgctttgaaaataattagttgtacagtaaagcaactccatatttggtaaacaatatttttaaagtgttgttaacacaaaaaaaaaaaaaaa

SEQ ID NO: 9 - protein, 6 aa, Artificial Sequence
  Description of Artificial Sequence/note = Synthetic Construct
SLKLGL

SEQ ID NO: 10 - DNA, 25 bp, Artificial Sequence
  Description of Artificial Sequence/note = Synthetic Construct
ggggacgatgaaaatgacgagggag

SEQ ID NO: 11 - DNA, 25 bp, Artificial Sequence
  Description of Artificial Sequence/note = Synthetic Construct
caatcacccggccaaggcttgcatc

SEQ ID NO: 12 - DNA, 22 bp, Artificial Sequence
  Description of Artificial Sequence/note = Synthetic Construct
cagccaccaccagcagcggcag

SEQ ID NO: 13 - DNA, 24 bp, Artificial Sequence
  Description of Artificial Sequence/note = Synthetic Construct
ggcttctaccagctcagctccctc

SEQ ID NO: 14 - DNA, 25 bp, Artificial Sequence
  Description of Artificial Sequence/note = Synthetic Construct
cagtcagccagcaggagaagcagcc

SEQ ID NO: 15 - DNA, 25 bp, Artificial Sequence
  Description of Artificial Sequence/note = Synthetic Construct
gctgcagctcaacagacggttcgtc

SEQ ID NO: 16 - DNA, 23 bp, Artificial Sequence
  Description of Artificial Sequence/note = Synthetic Construct
gggggatggtgaagatgatgagg

SEQ ID NO: 17 - DNA, 25 bp, Artificial Sequence
  Description of Artificial Sequence/note = Synthetic Construct
caagagagggagagagactgagagc

SEQ ID NO: 18 - DNA, 26 bp, Artificial Sequence
  Description of Artificial Sequence/note = Synthetic Construct
gaacagccagccatttctctctctcg

SEQ ID NO: 19 - DNA, 24 bp, Artificial Sequence
  Description of Artificial Sequence/note = Synthetic Construct
gtaccgttagccccagtttgagag

SEQ ID NO: 20 - DNA, 24 bp, Artificial Sequence
  Description of Artificial Sequence/note = Synthetic Construct
ccccgaaacccaccaacgaagatg

SEQ ID NO: 21 - DNA, 24 bp, Artificial Sequence
  Description of Artificial Sequence/note = Synthetic Construct
gatacaacacacagtcaccctccc

SEQ ID NO: 22 - DNA, 24 bp, Artificial Sequence
  Description of Artificial Sequence/note = Synthetic Construct
agcagccgccctttatctctctag

SEQ ID NO: 23 - DNA, 20 bp, Artificial Sequence
  Description of Artificial Sequence/note = Synthetic Construct
gtagtctccgtgcaggctgc

SEQ ID NO: 24 - DNA, 132178 bp, Artificial Sequence
  Description of Artificial Sequence/note = Synthetic Construct
aagcttgcacgccccaagccttcaaccaaaagacctttgctgcgtgccataatctaccgattttgaactccacgatacactggaattattacccctccaaaggcaccgtcgacgtggcgttcgcacaagccgtggtgagcgactccagatgggttgcatgggccataaacccaacctccactggcatggtgggctcccaagctatcgtggctttcaagagaacagacggagctatgtccgtttactcgtccccgatcaagagctatggaactcgtttggagcaaggaaatctcagcttccctctgtttgatgtctccgccgtgtacgagaacaaccagatcgtcatcttcgccaccgtaggccttcccaacaacgccagtgtcgtcaaccatgtttggcagcaaggaactttgtccggaaacacaccgcagatgcactccgtgtccggaccaaacgttcagtcctttggaactctggattttctttccgggaaagtggaaacagtcaggaggggaacgagttttgtattcagagtgaagatttctcatggaattattaataccatcagttggggcattctgatgcccgttggggctattgtggcgaggcatttcaaggctgctgatccagcatggtttcatgttcatagggcgtgtcagatgctgggatattttggtggagttgctgggttcgcaaccgggctttggctcggccataaatcttcaggggttgaatataaaggacaccgatgcataggcatcactctttttgctcttgcaacacttcaggtgttggttgctttgggtttgaggcccaacaagacagacaagaagagagttttctggaactggtttcactacttggtcggttatggaacaatcattctcggcattgtcaacatcctaaaaggatttgatatgcttcagccaggaaaatggtggaaattttcatatcttatcaccatcggtgttttgggttgtgttgctgcagtgttagaagcacgcgcatggtttctagttttgataaggaagaccgatcaagctgcagaacaaaataaggatgacacgagtgtagtttaaggtagtgtaagaagtgataattagctttacaattgtatttacttattttcgtgttagttatttacactttgtatttgtaatactcatttttaatttcaaggatcatatataatattttttgtttttgttaaagtacgtctcaatttgatgatatacgaatgtcaaccaatacctataaatttattttaactattttaacatcgttggttggaaattaccttcaaatagaagcccttcttctcaaacctgctaatgatgtcaccaatctgcaacaaacatgaacctctcagaaaatatgaaaactttcacataattaaacaatacgcacgaattggattaaaatagtttattggattctgtgtaagaaaaaaactatggccgaagttacaaaggacggcctttgttcacccacttcacacatccacagaagcaagattatgcatgtaaaatccaaccactacatactacaaatattaaaacttgatgcatgtaaataaaaacctaaaatcccagctcaggagtgttaaaatttgcttgctccccagtccaccaaaaagaaaaccaaccccttcaattcctaggttttcccaccaaccaaacaccataccagtaacacttgctatgaaattcgtaaacaaaaacaagaacttaggcaaacccactcaaacacagataagattttgcccagtaactaattaattactgtttttgccagctcagaattattgttttatctctcttttgccagtaactaattaattaaaaggctaaaactagttgaaggtgtcttccccttcattccagaattggaataaacaattatgtatgcttcttttgacatcaccaaagccataaggattatgcatatgtacactgcaagcctccgaaaaaaggtgtacaaaatttcaaccaaaattttctttgaatctgttgaagaagatttcattggcccgtactattacctcctcctcaatatcaaactcattcacatagtctaaagtagcgtccacccctacagtgctattccacttgtacagtcaaaatatttaaatgaataaataaatttataagcatgtagttgcagagcagaagatgcaaggattttctaagtgactttattagacagtaacattaggcttctaatactagaaagaaatcaataaagcagaaaggcatgggtgagcaaggcccaattttccagtccttcgacaactagggtttagcaatcatcttcacattcatcatacacagaggttcctccgcacccatgagagacaaagcttcccatcttatactattgctctgtctctcttctcttcttttgctctctccttgaaattacatggcaacccgttcgtacaattctctatctgtatttttatctgtaaatttgaatgcgtttgtttttcttctttgtgaaaatttggttttgttttgttgattttacagcgggtggcaagatcctctcttcaggtggcagagacagaagaggttgcttcccctctcagaatgttcagatcgaaggattggtaactctccatgattcgcgtcttttctctgttttgctgctagtaaatttttggaaaactatgcatgcctaagttgattataactatggacacgacccgcccaggccacctagatcctcccaggccgcctaggcgccgcctagatccgcccaggccgcctagacgccgcctagatcctcccaggccacctagatccttccaggccgccaaggcgccgcctagatcctttcaggccgcctaggcaccgcctagatccttccaggccgcctaggcgccgcctaactccctggcgtaggtgtcctcccgcctagtgcctaggccgatttttcgaacactgcttgaaacctggcacgggcttgctctgtgtcctagacgttcgtagtgacgagttcgagtcctctatccgtggctaccatggcgtccaattaacgtatgtttctgattttgcaattttgtttgtttgtttacatggaaaaggattatcagagctaaaccctggatacattaaagagtacagccaaccggctatacctttaaaatattcaaatatattcaaagagtatagccgcacggctatgcctctaaaactagtgttacaggtaccatctgccatttccaataggaattgcaagacaatcactctagcaataattatgacaataacaactttggacacttacaagtatatttttcttaaatggtgatgagtagaagtgttgatcaatgcttgaagctccctttaagaacctttgagtagaaaatataaggggttcttagatctaagtccaaaataattagttgtcattgaatttagtccacatgttttttgttttagatgtaagttctttgacttttattacttttttgttgtgtcgatttacctatgggggtaaataaagaaaacacattgaaaatctaaatttaatgcattatttcctcgtaattgaatttcataatttaaattttcaattctttccttggcaatatttacctaatatatggataaaatttaattaattcatgacagtgaaaatccacatattatactaattactcaggcaagcacccacatgaaattatgtacctaacatacaggtgaagagtgttattattagcaatggaaaaaacttagtgataaattgtgttacctataagtcagaaacataaattaaaagactacctataagtttggaacataaataagaagaccacctataatttagatacaaaaatagacaaataaaaattgtatgttacctattaaaaaaaaaggtacataaaatactattatatgttgtcgaaaatatgaaaaatacatatatataccatgcaataggcaagagaacaatatacctacaccatatgataatatatctaccagagtatgtaatatacctccaccatagggtgaacaaaaataaaatttgttaccaataagtcatgtgctactgtatttgcttccccaaaaacatgtaaaataaaggctcaagcaaattgtgccaaaatagacatgcagtcatccaccaagcaccatgctatgccaaatgaatttcaagagactttccgtatcttacatcaaaattgacatcaaaatggtccccaagaaaaaggaattgcttgctgcaatgaaggttgctgatgtgaagaagaagtgggagaacagctcttggggtatgcaactgattgttcataggagaagtgcatcttaggaaatatcatgatatttagtttacagttttgccacggattttcttttggaaggtttatgtttggtacagtgagggaggggtgagagaggtgggttggcacagtggggcggcacagcaggttggaaggagaatgagataaaggtttttatttaaatgctctctgggatgtgtttgtgatattttccttttctattcaatgacaaacgtcataagtggatgaatattatcacataatttactattactcgattgaaggcataaggggcaatattgaaacaaaaaataattttttaaaaaataaaaataaaaatacattagattatatttaagagtaatgctatttagacacacaacattgaccaccttaactgaccaccttatgtggcaactgatgtgtcatgccatgtcaattaatgaatgttgccatgaggccactcattaggaatgctagggttttttaatttgttttttaattgacatgacgacattcattaattgacatggcatgacacatcagttgccacataaggtggtcaatgttgtgtgtctaaatagcattactcttaaaataaacttctcaatgaaaatttggatttatatcaacttttctaaaaatataacatcctagttaatatacatagatcatttcaccagctattgaaacaaaactagagattttttccctattattgaggcccaaaatgtcaagagaagcccaagaatattcaaagcctaatataacgtccaaaagttgtcacatgctaatccaagctctatttcaggaatttatttccaaatgaatatacaaatatttttttatacatgatcaaatagtgtgcatgtcatgccaagcaaatgtcaaataactatgtgcaagctaatacatttttcaatgttaaaccacctaacccacttaagtaggttcacactagaaacatgcatgggacttgctgagcaagttttggtgtggatggttacaagtgtctataaggcccgttgaggggtcaaagagtggaagttttggtccgcttgggggcaccaaaactcagacccactttcatgagcccaattacaagagtaagcagagagaacaaccagcctcttctaccagcatcaaagccagagctacccacatctcttccacaagcattaaaacctagcttctaccatctcaccatccatcattaaaaactatatttaacactcaaccgccaacgtcttacctgccagtattaaaggccaaagacttatctttgcttctcctccagcgcctataaatgggaggcattggttactgagaaattgagtatcgactcaaaacttgacaaaaaagaaaaggaaaaaagagtatcgactcaatttatgcactagcggctagaccattttttattttattttatatttaaagcatatagcagctcggctatatccttttctatattagagtatattatatttatttttaacgagtaaccgggcggctttactctttttattttttatgagtatagccgctcggctgtaatcttttattattattattattatttttgtattgtatcgacgctcggctatattctttaaatatacaatttttttagatcttggggctaaaataccactttagctaattttgctagagttatttcttcaattattttgggcaagaataagaaaaggggttaaacccataaatacaataacaaaagggatccaccaactttttgtggatcaaagaatgttgaatttcaaagtataaaattgcttgaatgcgttttgaatctaaattttcatgtcctaaaaaaagtcttcatggtatttactctttttaaccccttccttctagggttagagtctgagaagtatatgtgggagagggaaagagactcaactcacaactactactcaatctcactcaaatacataaagaaggaaatatatatatatatatatttaaatagtatagccgtgctgttatactttgtaaatatatttgaatatttactgatatacaccttaaggtccaatgtattgatactttcttaaaactatctataaccgtacaccttaaggtccaatatgtatctactgataatatatcttgctttggttactgcagtttaataatttgcttagataacaatctgaaattaaaatcaatttcatcccctggaaagcatggtaaaacagatgttagcaagcaaagcatgaaaaagccacgtagtacctggagagtcctaggaactgatggtaactccatgtgagtaaccaagtgatcagagaaagatactcctatgagagcatataattcccatgttgaggtgttcccaaggaccaaccaccatgcttcgtttttcacctgtaaaagagaatctgaagctcagtttggtttctcattactagtttagaacaagaagaagtttttaccttagggaaacgcggggtaaacgctcttgatttgttttgcccgaaattggtgtttaccagtgtgatgtctaaagagcgagactttctgctgtccttatgctgaagttttaatttcagtttgatacgtggaaaaggctgcagatcctagaagaacaaaatattctagatgcaaattgcaatgcctactcaaatcagttaacatgcctgaaagcaaataattgacttgtatttcaatataacctacccaggccagtcaaaaagttgtccagtcatgttaaatcttgacctgttggttagtcagccatgtgtcaactatcttagaagtctatacctgatacaattttgaagcaggaaaattttcaatcatggtctgcaaagttgccttggggagatacaatagttgcggtacgctgaatattcctcttttgcttagcgagtcagcaagttcaacattcatgcatggcatcatatccataaggaagaatccccatcaaaccataaaccctgaaaagaacccaaaagatatgagaagagacgaaaactctactttcttaaggaacaaatcatccgagtcaaagagtaattgttaaaaataaataattagataaatcaaagcttcacctttttcagaaacaattagtgtaccatttaagtgcaagtgctgcacatcgtcctcacagacaaaaatatatatggctaaattccaaagtggcccttctaacaacgagtaagtcataatttctcaggctttaggggtctctgaaggtctggttaactcacatccaacccttcctcaggccatataaatctacatgagaattgagcatggtattacctagacaattaagagctgagaacttcaggttcagtattgtccaagccatagtaagctggattaaacatctgtagaaaactatgctcatgtaagatcttgtataggctcaataaatcaaatactaaggacaataatcaaaaccaaaggcttttaacttgtgtcttcaccatatttaaggacaagaggatcattgccaattgaagttgaaaactatgcaactttgcatctagaaaattgtaaatgataggtgttctcaacttttttatattctcatttcctaatgtgtttttcaggaaagactcatgcttactgaggtaaaacagaaatgcctgaatcttgttgtttgcaaaaagctcctcaaccagagatctatctttgtcagaccggcatggtgtaatccaatgccaaactgcaaggtgtgcctcaggttaaagaaccatctactgggcttcttctggcatacttagaaactgcctttgatgttcatctgactttgcgaacttcagtaacagaaaagcacaaaaaaaaacaaaacaacgcagcagccgaaaccgaagaaaaaaaatcgaagcttgagttgagaaagaggctcaagaccgagaatttcaattttgaagattggactgcgagtgcaagcttcgtgttcgtttctctttctcttcatatcactcgcgcaccacattcctctacgagtgcaagctttatctgtcttcatttggaaaaaaaaaaaaaaaggacactacccgcccaggccgcctagatccgcccaggcggcctagaatcctcctagatccacccagtacgcctaggcgccgcctagatcctctcaggcagcctaactccctggcgtcggtgtcctcccacctagttcctaggctgattttcgaaactgctcgaaacctggaacgggcttgctctgtgccctagatgttcgtagtgacgagttcgagtcctctatccgtggctaccatggcgtccaatttacgtatgtttctgattttgcaatttttgtttgtttacatggaaaaggatcatcagagctaattaaaatattcaaatatatttaagtatagccgagcggctatacgtttaaagtagctacactgttatactaaaagagtatagccgcccggctatactgttaaaatattcgaatatacttacagggtatagccgctcggctatactgttctccaacaacccattaacatttaggaggagaagcatccaaagctgaatctcttgtcttgtggcggtagcccgcccaaaccaaaagaaaggggcaaaacgtctctatccgtctgtctctggtgtttctctttaaacgtcaaacaagaagcagcaaagtcagccctccgcatgctgtgagtatgtgactgttggcttggattcattgaacttgtttgaaattgaaaccaaccatgacaaaagtgaaaggacatatacatagaacatagagagtagagggaagcctcagctgtgcaaattgcaagcaagaaaagaagaacggaattggagagaaggtgaccactttccataaatatatggggtccacgccatctttctttgatctcttctctgcactcctaaaactaataccagtttttttatgcccccactttgaccagacaacacaccatgctgctgcagatagaaagaattaaatggttccactagcctcctcttacttgtaagttgtaagttgttgttcatgggatgggatcaattacctctttcctccatcagcaacaaaagggtatctgcactgcagcctagctagctttgctttcacttccaagagccctctatcttatcatctcaaagagttcagttaaatctagcagagccagagagactacaacaaggtgtgctttctttctctcctatgtgataatgaccttccctcagtggagaccccaacaatacacacctacgactccttgaagcaagtaactaattaattaaaaggctaaaactagttgaaggtgtcttccccttcattccagaattggaacaaacaattatgtatgcttcttttgacatcaccaaggccataaggatgatgcatatatctacattacaagcctccaaaataaggtatacacaatttcaaccaaaattttctttaaatctgttgaagaagatttcattggcccatactattacctcctcctcaatatcaaactcattcacatagtctaaagtagttgcagagtcagaccacaaaaaaaaaaaaaaaaagttgcagagcagaagatatgtaaggattttctatgtgactttattagacagtaacattaggcttctagtactagaaagaaatcaataaagcagaacggcatgggtgagcaatgccaatgcgcctccctccactaaccgacctctccctgcatgcgtgttgacgactaatttatcttttccttttctttaccccaaccaagaccaccaggaattataaatctctcattccctcttcatttgcctcccaattctcactctctcaacaacagaaacacagttttctatctcttcctttcattccacacgaaataaacccctcatttcttatgcctttccttattttatgtcacctttcctctttctcacacactatacacgtcacctttgctccttcgcaccagcaatagttaatacaactacaaatcctctctctctcttgaagcaagttataggatcaccttttcagagctacacatatctcccaatcggctccattatcccatttgttattccatatggtcatgtgtgaccaaaaccatcatttgcaatatatctgtgattgatattggaaaatcaacatatctccttgttttcccatcataatattctgttccctgcaagagaatcaacacttacgtaaatttgtcctgaagaacagctgctgcaatatactcatgctagaaacctttgtaaatataggaaccaaatgaaaaagataccacacaaattttgcattaccttaataatgacgagatgatgacctggaaggtttacacctcatgccaatgtgctggtacaaaccaatacctataatttaatacatatgcctaatcagtatatagagttagaaaatgatcttttttatattctcttttcctaatgtgtttttcaggaaagactcatgcttactgaggtaaaacagaaatgcctgaatcttgttgtttgcaaaaagctcctcaaccagatatctttctcattcagaccggcatggtgtaatccaatgccaaacggcaaggtgtgcctcaggttaaagaaccatctgctgggcttcttctggcatattaagaaactgtcttgcatgttcatctaaggttgcgaacttcaataacagaaaagcaccaacaataaaattgaatcaacgcagcagccgaaaccgaagaaaaaaaatcgaagcttgagttgagagagaggctcaagaccgagtatttcaattttgtagattggactgcgagtgcaagcttcgtgctcgtttctctgcctcttcatatcactcacgcaccacattcctctgcgagtgcaagcttcgtctgtcttcatttggaaaaaaaggacacgacccgcccaggccgcctaggtccgcccaggccgcctaggcccatcctagatccgcccaggcctcctagtcgctgcctagatcctcccaggcagcctagccgcaggcctagatttccccagaccgcctagatccgcccaggcctcctagatcctcccaggccgcctaactccttggcgtcggtgtcctcccgcctagcgcctaggccgatttttcgaaactgctcgaaacctggaacgggcttgctctgtgccctagacgtttgtagtgacgagttcgagtcctctataaccaatcaactgatggttttggtgacgagttctgtgctctgtgcccgcgcggctatactctgtaaacattcaaatttactgaaagcgtatagccgggcggctatactgtttaaatattttaatacgtgttaatagttacgccgaacggctatacttttaaaatattcggatatatctagacatatactctttaaatagatttatttatcgggtatagccgtgcggctatactatttaaaattataattttcttttttaaaatataaaattagtttctttaggtcaataaatagttttttttgtgagtaattaatattaattattttaattaactccataaattatattacttaataaatagtaattaactatatgatgagtaatgtttaatatccaaatcaaatttgaagcctaattggcttctacttaacccaacttccaattactaagtataggactaccacacttataaagacttcactttttcatctttctactaatatgggacaaaaatccttgaatttcattcaaacttccaacaagtagaactataaagcattagctatgatccttcttgaacaaataagcagtttatcattaacacctataggtcccttagtgctgcctcgctgcaggtccctaaagccaatcaactaccactcccctatcttcttttgcagtaataagactcatgtaggtgtgtatatttttttgtggtattattgagagcaatgttatgtgcgtggcaagagattttctttgacctttatccctttatatatacaaatagagataagatgcctagtatcacaaagccatattgggtataatagtgaatttcccttaaaacaattgtttggtttgtattaaattacattcgagtacatgcaaatgtgtagtgagtacaagcacgcaatgcggctatgcaaatggtttcatctttagttctttttttgaatataagggatagtctaaattgcaaaggggagggaagtttctctctctcacacacacacacactaccaatgtgctatgtgaattcgaacctgagaccactagtctgcaagtcaagacccctttctactaagctacatcccgttgatctttagtttttactacataagaaaaaaaaaaaaaaaagttacaacatgcaagttatgatgtgatttaccccacccccttacactttctccccataattattaataataattatatttaatggaagaagcaagaaatttagccccaaagttctcgatctttgtccaagtatctgcctcctccaacaggtctgataccattcatatcaagagtccatttctcaaacgtacacctcaagaaatcataatatcctccatgaagagagagcatctctttctttgctctgtcttctatccatgggtatgtccgtaaattcaataatgaactattgattgattcctgtaatagaatgaaaatttttataataccccaaggatgaccacatttaaagacttgctctatattttttttaaaaaccaattggtaacgagtggaaattgcatgaatcttttaatattctcaggcaatacttttaattccccgtcctgggattcacactctcaatagaatctgttttgttgaatcgccattagttagaaacaatctattggtcgcattttagagacgtttaaaaccttcctcttagaaattaatcacaagtaaataagcctaaaaagagtatcaagtggaaaggactcaaggagtaccttctcacagtgtctgcattgctgatcaaagctaaggtgtggtgcaacagcttttgttcttagtttggcaactttggcattgataacccaactatgagttaagctactgaaagcagaatcacagaaaaaaaaaattgcagcatttaaagaaaattgatgtcctgaaaataccatgtgaacaaaagtgagaaaggatcaattgttaagaaaccttgaatcaccatcatcttgcattctcatgagggtctcaattcctgcacagctactgtggccaatgactaatatattcttaacctgaagtcagccataaacattttgatcaattttcaaggtcgctagctccataaccccgaatttggagatggattccgattttaagcaaagcacagttaagttatacataaacacagaggttgaaagagattcgagagcaagatgacatacttcaagagtatttactgcaaactcaagggcagcattagtttctgatgcttcattctgcaaaagacaaaggttgatttgaaggatttgaaggattatatttcagactcgaggtaatatgtcacgactcagggaagcgcacgcttaatacatacctcaaatgggggaacaaggtttgctacatttctaatcatgaatgcttctccaggttgaaagcctaggatgttagagggacataccctagagtctgcacaagcaatcaccatgaactgtttttgaacttgtaagtttatttatatggaactgtccaatttcaagctgaacagaagcaagaagtattaaaaacaagataaaaattaaatttgtataattaccttgggtgcttgagcttgagctagagtttggaaatgctctgactctttcctgttcaatatagtataattaccacacagaccaatatgcaatcaagttgtgtgtaacattaaactgctacttacaaaaatttgtgcttcttaaaactcagaaacctctctttcatctcaccaaacaagtcaggcccattattagcttttgacacattttctccttcacaatttgaacattcatgggcaagttccatggaatcacttgaagcctccagccttctaactggttgtcttctgcaaaacgacagccgatcatagcagttatggtggagctcttagtggtatcattatatatctataagattaaaattcaagagtttccctagtcatgaatttctgttttcttttttgggtgaaaatgaatatcctcttttcaataaagacagattaaatcatatttcttgtgatccagatattcaaacatggaactaaaataatagaggttgtgaggtaaaaaaaaaaaagaatgtgacatttcaagcacaataattcatatcaaagagcaatgattggaagatatacttacttgactgaaggcagtaattctacatgggttccttgttcaacttctgcagaatttgtctaggaattttcacatattaaagaaacaaaatctgggtatcagtagtgacacaaccgatttgataataaatatatatattattgagagcttgaaacaagtaccaggtttgaggcagaggattttcttctgtaattgctacaggttgaggaaggtcttacagctgccattgttgaaagaatccgacgagcttttgagcctgcaattgtgaaaacaaacaaagattcatgagtttaatgatatacaggtaagttggttggttggtttctgataatacttacggattgggaagaccatctctgttcgttcccgccttcacatgcggtcggtccaaaaagatgagtttttagttcaagaaataattttttatttttttattataatgaaatgtgaatacaatacagaaaaaggcttcaatcttatccaatcccgacctgtagtcatccagagtttgtaaagtgacgtggatatgcctatttggaactacatgatttttgaatgggaaaaataaatttagaatctgaaaaagaaaattctgaatttttaaattgacatacaccaataatcttatttgaattcataaaaaaaaattaaaaattaaaaattaatcatatattacaattaaaaatttaaactaaacataagaggttatagtttgattgtttcagtctgcttctttttttcctcaaagagaagaaaaaaaaaaaaaatctgatctcagtatatctcatatatataaatccaaaaacaaaaaacaaaaaacaaaaaaatcctactgattttattccatgtaatccaggatccaggtttctgtacaccaacccaaaatatgagtttccctttttgccctcactacttccttaatattcattctcatcctcataaaccgacccataaacccgagctcgctttttccccaaaaccaaccgacccggcccacaacctgaggcccgattctccaccacagccttcaaagcccgcttcaaagccctaacctcctcctccccgccaccgccggcccattgcatcacaaccacattcttggtccgccccccaacagtcatcatctcggcccggatcgccctggcctgaacggaccggatcgcctggatcaagtcccggttcagacccggcctatcctcgcaacacagcgtcgccttcagaagcctcgcctcgccgtcgcaatagctcaccgtggcctcgtccgactcgcccggaaacggccagggctccgaaccggtgctgccaccgcagcacgagcccccgtcctgtcgcgcaaggtcaccagcctgcctcctcagctcccgaacgtgctgcaccacctccgctagcaacgacgccttgtccgtctgcgcataaaattaaaatcacgccgagtacaattaccataatgcccctatattgaaatcgaaataacgagaaagggagcatgttttggttttttcacccgggtggtgttggggaggagagtgcggagggtggagaggtgggtgttgatacgctgcctgcgtctcctctcggcttccttgtggctcttggatgcttctgctgacctggaggtctcggtcatggaggctgctgggctcaggatggaatctgcgccgtccatcaggttgaccagcgacgggtcgtgtgcgtagtgattttgatcagaccagctcaggaaaggaagcattttttttttttctctcgtaatgaaaatcaagtgtttgagaataagaagagttgggttttgttgggtaggtgtagttatagggaactggggtgtactgagagagagagagagatgggtttgaagaagaagaagagagaaagtgagaggaagaagagcaagtaggagttgggagaagtgaagtgaacaagaaaaacagtgagttatttgacttggtttttacttttttatttatttaccgctgatgggttgatgagataataaataaataaataaaatattaaaatttaaacaagcgaacatgaatatagattatttagataaataaaaggaaattaaagaaagaacatggaaaacgtgaatcgaggcaggactagaaacttggagaaagggcaaaaagagtgaaagaggggaagggtttggaaggggaccaagggaatattgttaaagaatccgtgtgacatgtccatgttaccaagtatgtcaattttattattatttgaaagttcaagcatgttacattgtattatccacccaaaaaaataaacatgtcatattgtgattaaaataaaatcgcttataagttaacaaataaaggttaacgtagtaatacgtacttttcttgcgagtatttgcgcatttgggaggagatcgaatccaagacctaatcaactttagttttttcgaagtcaagaagaagtaacttttgagcttcagtaaaatcacattaaagttaacaaaagtttaactagtaatggatttgttgttggtgctcaaaactagatgatcagtagtgggctgaaacttaaagatgaggtgtgatctgatttgcattcagtaagaacttcccttcggcaagctgaggattatctgcacagagagagagagagggaaggaaagagcagtagataagacctaagacaccggtgtggtgccagctgaaagtcctttgatgctaaagttagccgaagaatagaggagttagctattgataaagtaaaggcataagtgtcaagtagctgttaccttgtaccttggaggcaatggactctatttatagaggtatttgtgtgtcctcttgatgtaaattttcgatgtgtgacttgtgggagtaacctaaaaggctgccacatgtcctagcgaatataccagtaaagaacctgagagattcctacaagataccttctgggaggctaaattgatagaaggtgccgagggtacctgccgactgtcgattgcgaacttatgcactgggccagccaatgtggaattggtcgtgggtgtcgagggcaccaaccgattgttgtaaggctgggctggttagtgtgggctcttgggccctaattgatgtagaatcagtaagtggtgtcgaggggcgactaccaattgtaatgccaaataggccagcatgggctcctgggccttgcttggtgtcaggtcctttccacgtgtcatggcatgaaaggccagtcagatatgatacaaacatttgtaaaaccccattatctaacccacataatcttacaaaattgatgtcataattgtgattaagaaaatattttatctgaacgagcaatgcatatttggtttaatagtttttattttcgtttttacagtgatgtagctttgttgtaaggaaaaaaaaccattaatattaaactcaaaatttaaaaagaggaaacacatatataaacacaagataatgtagaaaaagaaaggagagtacaaaattgaagttgatagctcttagcaattaatccaataaggttgcgtgaacggcggcggcaacagctttttttaggagaatcaaaggtgacagcagcgacatcattagccgacaacctcttttgccttcgccttcactttccagtttcagttattgcttttcttgcccttggtttcactttctcccaaccttttcctccttttgctctctctctctctctctctctctcctcttcttcttttttttttttctttaagaacttactggttttcaaaaatagcaaggatctaaacaattggagatgacaatagaaataacctaggatcctagattgcatcatcgactaagaaaaatcaatcaaaatgtgataataagaaaaacgtggtagcaaagaatatacatttgaactaagtgcaatttcctgaatgtttgatcaatcaagcatcatcagcatcgcttccaccaatctgagtttccagagttaaagtagttgcttccatcaggaccaagagtatggaatgtctcaaggataaatttcccttctttgtactacttctggcttttctcccttgcagcctcatatttcccaacccaccattgaactacacgaaacacaaacagtgtaatcgtgttatcatgatccactcttctctctccaagtgtgacatttacaaatttatcaaaaagataaaccctttcatgtcttcggttaaacgacttagaattttagaaagtatgtcatcaacaagagcaggatgagttttgcagtgcttgcagctagatgtctccttcaagactgatcccaaacaaccttcccatgtcgttactttagagtctacatctttagctttcttaatttctcttcccgcagttacaagggagcataatagcagcaaaatagattctgtgttgcactctcctattttattttattttttctttgtctaattgactttcttttatttatttatgtgagttttagttttgagggcatttaagtctatttaattgagtttttggctatttttaaaactgacatttatgaaagttgggcgtaaattttggctattttttataaaaactcatttgagaagggggggctttcgcatgcattgccattgccaatatgccaaaaaggttcaaatctaagattattaatatgcaagtcaatgtctttttcaactgggctaaaccccattgacatgggcatattttaatttaggtcatgttgcacaatttggatgtatcacttcgttatgttacgtctatcaaatcttagttcatgtgtgaagttttcaagggtaaactgtcttttcaaaacatgaaatctcatttaacccttatttggtcactaaaatttaaaaaaaatgatttaatgacgctaaaatattgtttaagtttcaacaagattcatttaaaattgaagaaaatgttctactttatatgttcaacgttgggtaataatttgtcattggtgacgtgaatgtacattatcaagtgttatttgggttcaaactgaccattggtctgcaagtcaatgccctttccactcaactagccctcgttgactatgacacaactgtattttgagtacgcatcccacaatattttttttctagatattaagacgatattttactgagcattaagaatcattagggtaaaggaggatcatcattccataacataataaaaccatcaataaatgttaccaagtttcaagcaatgagcaacctcggtacactgttgttcaccccaaaaagcatagttgtggattttaaagccatgaaattgattgaccatagccaattttcacccaaacaaactgtagagaaaagtatgatgaaggtcacaaacaaaaacttaaatacactacactaccaatatatctgatcattttggtccctaaaattggagaaaaagtttccatggcatggtctgtgttgtctcttctctccctgtcatcacttggaaaaaacccttcgtttcttttgccttaggttttaactgtgacccaatattagtacttcaatctgtccagaaatacatcaatttttttttcttataaaaaatttaattaaacaaaataaaattccaaagattacccatcttgtgaggactgaggatgctaggtggaccccacaaaggcatgaattgaaaacgttgagggagagaggaagtaagatcaacatcccatctcagtagggcagccgaccaacaagtgctgagagttcagattgtgatgcagaagagagcagaggttgcagtcataataaaaatttattcactatgggctatatgcaaattacaacagtttttatgaacatgacatctgcctataggctctctgtctctctctatatatataaatagtgattccaccatatttcagagcatgccatgcctctggttctggcttctggcttctggcttctggcttctgcctggaccacacaccctaaaatggcttttgtttgaaccccattatatttgtgtaggtcctgctgacagtccaaaacctagctataagttttttggctttgctaagagctaaaagcttgaatgctaacttgagccatgccaatgaataaaggaactgcagcaactaataacaagagcaataaagaaaccaaaaataaagtagccaaggtaaaaaacaaactacgcaggtgtagcatgttttcaaggggcaaatattggcgtatgcatggaatatgttaatgagaatgataacataatgatatagtttatttagtttataaaaattacaatttatggtaatgtataaaaccacaatttggcagttagtgtaacattccaattccctgaaaactgtgctaatttattttaactagttatatattgtcacaagagaacacaaactatatttcaaaggttaaaatagtaaaaatcaaaagaactgaacattcatggttgaatgttgttttaacgttaaaacaagtgtaccgcatcgttattggtttaatggtatttatcttcccaatatcgaaatagttttttcaacaaaaaaaatatttatttatttattaaaaaatattggaagaaatttcaagtttgaaacctcctaaccatcaaaaagattaaatgacaataaaaggttagtagaagacttgttcgggactatcgtagtactaagctaggcctccggctatctgtagtttatcggcaccctaaaaatcatgcactcctccctgaatttgacaataagtgtaatataagccaagcctagcattgttggctttagttcattgttttgctctcccaaccacttgggagaagaaaaatgtttaattgcgttgcagatcaatcaaggatgcttaaaatgtttagcaagtcagaaagaacaagttcaaagaagagcttaaatctccaactcaatggtttcatctgatcaaatgagacggatccaggatttcaaaatagggtagactagatttaccttattgaactattagattttaggcttaaggaattgatcatatatataaaatttaagaacatataatttttattttatttgtttaaaatgcctattttacaccaacaatgccaggtaaggtcaaccatgatgaagaaaaatgaattaagagaagaaaattcataagaaaattcaaaaatgaaaagttatcctcacaaagatagagtcagatagacagatgacaactagaggtaacgctacaattatccaattagttcaaattaatatttctgctataaatggtattaacattaacactaactataagaaataaataaaaaaaaactcacctgggctatagcccagggtgtacaagtcataggtccgtgtgatccaaaaaataaaagcgtttgtttttcaggccttaccagtatatctttcttctttttggttcaggccaaaagatagaattttttttcctacaaagaagaatttacataagatatacataaaattctgcatttcaaattcatcgctttgaaagtttccttctacattcaattgcttaacccagaagcacaaaaataaactgagcacaacccactagactttgcaaccacccataaacaaaaacgagcaaaaaaatccactagccgagaataatcaggcttactttgtcagtgaccttacagcacaaacctatgcatcaactatgcaattcttacatcaaaagctaacagctagcagcatctaacgacaaaaacaggttgttaagtctgaatggttctggccaaaattgaagtatctatatggcacatcataatagctgcaaaggaagagaaaagaaaacttagcattcttaacagataatcaataccataacaaacaacaacaatgtaataccttaagtcagacaatgagtccatttttgtggaacggagatcctgaaggagcaatcaggccaagatgttgcatcactcttaatctccgagcactcacttctgcctgatcacttgagccatttcgtttaagtttcttttgcaaaccatcttccctaggggattttctttttgcttcttcctctttggcaattggcatatttagctccagcttactttgtggtatagtgacatattgctgtcttctcggagaaacccttacctaatagtacatttattggataatgcaaatgcatgacaatgcaatccatgaagataagcatgtacatagaccagctaaaggaactcacagaaaattcagttctctgaacctcagtagtctgcaaccaagtttccgtatgtgacatcaagaataagcaatttacagaacaaggaccattgaaaatcagaacttactaagcttgacgaatctgtttggtgattttcccttgcatgattatgattggacacgcatgtaggagaataaactgctacactttctgctccataatataaacattttgaccaatataagaatctatttgtgagaaagagtagagtactttcccttcaggtcatttcaaacacagtaatttcataaacatcaaatagtaaattgctgcaataactggagctgtaaccttgacagaaacaaaacttgagctatgacctctactcacacacacaaacagggaattttggacatggaatcccatgatttacacaagaagaaagcaccatccgcctgttcgaaagcaccagtatagatgtaatctagccatgttctctatatccttctcaaatttatttaatatgaagtcttgaaagcatttgacaacctttcataattgtattttctttaggctctttagaagatgacaacttagcataggatctgctataaattctcaagacatccgtcaattttaatacttaaagcctttaaacttagataataatcaaaattcggtttaactgatacatattatgcaatacgttttaacagggattttgagttggctaacctttagactcagacccttgcttctttgatctattggattttaagcaagaaacatcctgtagacatatttaattgatataggagaaaccatcaactgcaagcaaacgttttcaatcctatgcaagagcaattatcaaggaaagaatagttacctgctccagagcttttagaagtatttccctataacaaccattgttctgcttgagtatctgaaagttcaacaatcaataggcataaaactattataatctggtggaaattaatgctcaaatacatcagcagaatttaaggagattctagaatgcattcaaaacatactatgtcaaattcagaatgatatttcagagggtccacatacaagctgcaagaaaacacgctcaaaagttatacgtaacaaggaatacagatgaagacaaaacacctgaccaaggaaaaaaagaattactatttatagcttccatagagccgaacaagaaccttccctgctgatcttttacttggcttgttgttcacattcacagtattgtcatcaacaacctaccaaataaacaagaagataacgatgtggattcttgaccatgacgaagtgaacatcaaatagaatctggtggtatcagcaaatgaaattaaatagcttgattactgaatgccacaacaaccagcaaatacagcatctcaaatacctgtgcaggccaccatgagcctccattgatcttcacccatatcagatctggcaccttaactttccctcccaattgattagcagcaacatcctcttcatctttcacatcaatacgatctttcccagttgaaaggcgtttgcttttcattgtctgccattgcagttgttaattgaaaacggaaaaaaacaaaaaacaagacaaaaacaaaaaacttcaaagtaatttcttgccctacaaacctctacagataacaaaattttacaaatggaacacgcatctcggtttcaaacaattatgcacaaacaattgtcacatgtactttgctcttttttttttcttttaatgaaactcatgacaatgcaacccatgaagataagcatgtacatagaccagctaaggaactcacagaaaattcagttctctgagcctcagtagtctgcaatcaagaataagtaatttacagaataaggaccattgaaaatcagaacttactaagcttgacgaatctgtttggtgattttcccctgcatgattgcgattggacatgtgtgtaggagaataaacagctacactttctgctccataatataaacattttgaccaatataagaatctttttgtgagaaagagtagagtactttcctttcaggtcattcctaacacagtaatttcataaacctcaaatagtaaattgctgcaataactggagctgtaaccttgacagaaacaaaacttgagctatgacctctactcacacacacaaacagggaattttggacatggaatcccatgatttacaaaagaagaaggcaccatctgcatgttcgaaagcaccagtatagatgtattataaccatgttctctatatccttctcaaatgaagtcttgaaagcgtttgaccttaccctttcataattgtatttactttaggctctttagaagatgacaacttaacataggatcagctataaaattctcaagacgtccgtaatacttaaagcctttaaacttagataataatcaaaattctgtttaactgatacatattatgcaatacatttttacagggattttgagttggctaacctttagagttggacccttgcttctttgatctactggattttaagcaagaaacatcctgtatacatatttaattgatataggagaaatcatcaactgcaagcaaatgttatcaatcctatgcaagagcaattatcaaggaaagaatagttacctgctccagagcttttagaagtatttccctataacaaccattgttctgcttgagtatctgaaagttcaacaatcaataggcataaaaggattataatctggtggaaattagagctcaaatacatcagcggaattcaaggagattctagaatgcattcaaaacatactatgtcaaattcagaatgatatttcagaaggtccacatacaagctgcaagaaaacacgctcaaaagttatacataacaagaatacagatgaagacaaaaccctgaccaaggaaaaaaagaattactgtttatagcttccatagagccgaacaagaaccttccctgctgatcttttacttggcttgttgttcacattcacagtattgtcatcaacaacctaccaaataaacaagaagataacgatgtggatccttgaccatgactaagtgaacatcaaatagaatctggtggtatcagcaaatgaaattaaatagcttgacgccacaacaaccagcaaatacagcatctcaaatacctgtccaggccaccatgagcctccatcgatcttcacccatatcagatctgccaccttaacttcccctcccaattgattagcagcaacatcctcttcatctttcacatcaatacgatctttcccagttgaaaggtgtttgcttttcattgtctgccattgcagttgttatataaaaaagacaaaaacaaaaaacttcaaagtaatatcttgccctgcaaacctctacagataacaaaattttacaaatggaacacgtatcatacaattatgcacaaaaaattgtcacatgtactttgctctttttttttttcttttaaagaaactcaagtcaactgagccactcatagtgatttgatcatactatggtatagccttttctatgttgcccatctacttttgagttattgttagacaattgataaaaatcacagcatacagaaagggataaacagtgtacagaaacatataagtataatggagaggtgaccttaaaaatgttgagacatgaaccagtgtgtggacacgcttactaattctgaaccaacccaatacgatcaccaatttaaccacctaagggagtcgagtatgggcaagcccaatgtaccaatcgtgtgtggggttttatctcaaaagtcatatgtgctattagactattagaaatggatcactcctcaaaaattgtagattgttgtgtcttctatctaatgggactaatccccattctcactcatggcttgcaccgagccacgtaaccccaacaacaaagttatagaaagggggagtaaggcagtcccaagtagcatgacaatgaagtatctaagtgggacatggtatgacacgagacagaagactcttgcattgtggaactgctatggtcgttcacataataactataattcatgagtctttgacgtaagcattgtgtaataatctaagattttctccaacctttagacctgcatccaggggctgcaaaagttcatcatgctttgcttcccaagttaacatgttattaacaagattttgtccatttagtaacatgttagttaattaaacgtgtatttaaatgtgatggagttagaaagaaagtttggatggtggtatataattaccatattggccttaattttaaagtttttattacactgatcaaaagtaataggatgtacttggcatttaaatataatggtgacaagatgattttcatactaaagattctgttgagacaaccatgcatagattactttgctaaaacgtaagtagtttgtatgaacgtttgaacctcattcaaagcttagaacttgatctcaccgtcatgttctttccttaacaatttgtgggaataaccattatcagattaccttgctaaaatataagtagttttctatgaacatttttacatcccattgtcaaatttgaccctcgttttcaaagcttaaaactttgatgtcttccttccttttgttctttcctacattttctcagcaaccaaacagaacaactcggaccatatgacaaaactagtacagtttaagtgacaacaaagtataaacagccaaagcagaacattcaaacaaaagcggatttgggttcttacagtttcaccagagacggcatagcaagagactgagattgagaacgaccgcggcgttctgtgtgcgcgaaggatgaacaggaaatagaaggtctgagtgggcggagaagtgtcttggaagttggaacgacaaaaccaagcaactttattgggttccttttctgtcagactgtatacgtttggtttttatttttttcaaatgtcgaaaattagaggtaattgaaggaaagaaattacgtgtaacgggaataccactgtttttttattcttatccaattaagtttcgtcatgtggaattttcatcacgtatgacaaaagatgattggttggaaatagcaatacaatattattttcatgatatacaagttttctcatattgaagcatgtttagttattaagaatctaagaagtcatgaattaagaatatgagaattaaagaattagaattggatcaattactctcctagttcatccgattactgattttgaaggtcaatactaattttaatgtgcaactcacaattttttattattttttttatgttaataaatacatgaaaagttataaattaaaataattccaatacagatgtgtttataaacatataaaaattataaattagaataattcaaatactcatccccaataaataaatatgctgttaactttttgatttttccttccgccataaaattataggcatggagtaatgtccatagtaccttttaatttttttggatataatgtccataataccttgaacattgttcgaatctatcaagaagtgtttgatccacgaattcacaattcaacggttatatttttgagactaggtggaaaaatcttttgtttttttatcttgtgggttggttgggcccgacctagttctcatccaagtttttatttcaattcaacccgagttggatgggcccaaaaacagtacaaaatctggtactcaagatccatagggtttgggcttgagtttcggccgagctttccaagttttcttttatggtgctaccctaagcttcaagctagtttagggttccaaaaaaaaaaatgtgagcttcaagctagtttacaatgagtgtgtgtttgacccaaaaaagaagaagaaagatgagtgtgtttttaaaaaatacatgacatgcgaacatggtttatatttacaacagttgctagggctccagctttttaataccaattgatgtatcacatgatatgacaatccatatcaaattacaggttattcattttagtttagtcgttttttttatattttttaactttatttgattaagtagaccaaatagaccaaaccgtagcttcatcctagccgcaacattgtcatcgatgtagtacttgatccgccgttgtcaaacgtccatagagccaatatgaatttttttttctttgctggttccccacgtcctggaaatcccaagttgatttgagggcatataacattgcaatgtgatggagaatatgatagaggaaaaatcaaattacctcttccaaacttggtcgtgacggaggatttgccctatatttgggtcaaaatacctcactctttctttgatttagttctcataagcgatgatatacaaagtttaatagccacaattttgaccccaaaaaaaaaaggagtttaatagccacaatgatgagatgtgaaattccagatcaagtccgagggtggatccagctaaaccctactatgggccataaccctcgctcactcaccctttgctatacatagggaataaaggcccacacatttcttcaacaaaaataaaaacaagcacgcataatagaaatgaagcttgtccacctttaaaaaacagaaaaattaatcaaagaaagaacatagaaggccaaacctttccacaggaagattctccctttctctggatctccatagttgggccgccaaggagtggaagacatcaattcatcaaagcaaattcattgtttgttagaactttttgcttctttttcaaggttttttttataccacacagaaaaaatcctagccgatcaagtaaactcttctatgttgtttactagattcgtactttttgtctatttagtttcctagctcaaacaaacttcttgcgacgggaatagtagtttttgctatctctagccaataactcaatgacacgcggaattgcatgaaagtctctctttccagtttgatgtttattaagtttgtatggatctctgtgcgttatttctctttattcaagcaatggctattttataggccaaaagatacaagaagaactgaactgaggagaagaaagagaaacatttgatgggttgaactcaacttcataaagaatgaaaaacaaatcaactttgttaaacatcattattgtgatgttatgcagaggtggagactggtgttgctggtggtggtggtggtgggataacttggcagagagtaataataaaattgaacaaaattaaaaaggtaatgtgacacatgacatggtaattcaagtcatttgtcaatgtcacataagttgatatgtaagttggtacctatagcattattgatatttacaaaaagaataatactagacatataacatttttataccattttttaatatcatattatgtgccatctcacatgataatccatgtcaccttccacatgatttattttagtttccatacaactttttagttttatcgtgtaagattttgttttcaggtaactatccatatcaccttccacacgaataaaataggcatttcatgtgatttggacaaataggtgacgttatttggagcatctaaataggaagttcgtgtgcttttacctcacgggagtgttatttgtgggcacccttagatatttgacaataccagtgattgatgggtttctcctttctacattactattgtcattgacgacccatttgaaccgttggtggtggtcgtcttcaatcacgttggtgagaaaagggatgatacgggaggagtgagaaagagacatttatttatttatttatttaaagaaaactgacatttgtattcataaagtaaacacaaagatcatcaactacaagggtaagaataaatacaaatacctacaaatgggtaatacaaacatgaaagcatagtaaccacatagagttaatcatgatacatgtcacataaggccaaaaaaattcggcataaaagtagcacgcctgaacaaacctgatatgatcgcccaaatatgccaatacaataacttttaaactctcataagtacaaaatacaagtggagactagattacaatcaaacaccaaactcacaaggagttgatgcaattattacaaatcaagaaaactgtaaatacacatcaagaaaataaaagttgcttagccacctcgtctatatccaccaacatgactaaacaacaaagccatccaaagctggggaggagagggaaaaatgaggggaagggcgggcaagaagtgaggggagaggaggggagatagaagaagggcggaggaggagagaagagggaaggggaggggcatatttttggatatatatattaatttgttcactaaattaaaaattaaaaattaataaatatgagttgatgtggtagatgacatgggttattatgttagttgccacataagagtttagaattattatgtcaactgccacataagttgatattataaagtggtataaaaacgtagcatgtctaagcattttccttacacaagcgacatacacacaataacttttccaaaatatgacggaatagagacagattccaaagtcccaatgaggtcatatcataatgaaaaagtatgtttggcctaagatgagtggtttcagattcaaaacctctagacacccatgtgtgagtatgtccccgccccttcctacctttgacaaaagaagaagagagattccaaaggttgagttgtcatactgtcccgcgaagcattatccaagagtagatgcttgggaataacacaacttattggctcttttaagataagaggaccatatgcctgcatagcttgttggagcagattatccaaactcaaattaaattacactcaactatgaacgaatttcatgtttttatttatttatttatttctattgggggggaacaataggtgtgtggagtataaaatacagactctaactttctaatctgtaagttgtttaagaagaagaaaaaaaaaaaaaaaaaagaagaagttatactgcactccttgtgttcagaataaccgcgaaaatccttaaatcaaggatcatccggacatgatcaaaactaataaccaattatatatgaaaaaaaaaaaaaaaaaaaaccctttcatatcagcagagcaattgggtgtctcagttccagttttgcgatcattggtgccaatgagtaggacctgtaatctttgcatgcacctattttttcttggttcacaaaattgatcaacttctcctcataaagtgaacaaatttgatgccagttaatcatccatcctaatttacttcatcccatttgtgtatgcctcagcgatgaagaatttgcaaaaggatcacattcacctttaaatgcgaaaaggaccattcacacaaaaaaaaaaaaaaaaaagtacaatgctttcctacttgaatggaaatggacatggaaattttctctaaagcatgtggtcccttgtaaaagtacaggcatttgtagggtataccattagtccagaaccaaatcttatcatgttgtttaaagcctctttttttccagtggtctctggttcctaaaatcgcagcaattgctctgagaagtattacaaggaaatttgttagtacttagtaccaaaccaacaaaatactgccagtctaagaaaggcagattaacatcaattctattctaaatttctaacaaatttttccccaaagggagtaccaagaaagattatgtatccaccggaaaaccttagcctccatatggacggaaggattccttagactagtacaagaatggatttcaaatgattccttaatcacatgtaaccttaatctaatgaggttattctttgttagcaaattttaccttattttcaaatccaaatcccttcttttatatccctccatccaaacgaagcctttgggaaccaagtaaggaataacaaccatgcaacaatgctatatattgctataaattcagtacacttcagcagctgcatcaaaaacatgtgcagcctgctgacactcatttataacaggggatgctgtaactgcaggtgcaactatacaagtaatgatatgaagcttggagtgcaagattgttttacaagttcaccaagacagcaaatatggatatgaataaacaatcagtcggccggttatccattagttaggattgctatctgaagcctgatcaatgaaaggcaatgagtttgtaacttcttagcccttcctgccaaggcatcagaaaccgaatttgcttgacgtgtaatccaacgccaaacacacgacctgaatctacgcacttcccggattctcatcgtcaaaagaggatagatgctagctccacttgccttttgagatatttcccttcccagtttgaattagctccttggagtcagattattcgataatgacatctttgccacctacattggcagcaaaatagcaccccttgagcgctgctgggggcctcagcccgacaaacgctccatgattgtttcaaatagcaatgccaatcctagctgcaaacttgttcttctcaccaaaggcataatgagatggcaatggcggagaaggacacgtttgcagctgggattctcaggcaaaatactgactttttaatactaacttacgtgcttagtcagcttttttatggtttctaaatttatttgatgaagtcaacacttctattttcttgattataattatatttttggtctatttattagtttcccaggtttgtatggctctctctgtgtgcgttatttctctttcttcaatgaatggatatattttacaggccaaaagatacaggaagaactaagctgaggataagaaagagaaacatgtgatgcttgacagagataataattatgaaaattaaaaagatgatgtggcaattcatgttggtgtgacataaagaataaagagaaattccaaagcttgaatcgtcagactgtccataaagcaagcattatccaagagtcttgggaataacacaacttattggctctcctaagagcacttccaccagttgactcttgccatggcaagattagccctagggcaggcactattcacgtgaatagtggctgccctagccccctccagtaaaatgtgtttccagcagttggggcttgccatggcaattactattcatttttttgtttttttcacaattttttttgcttaaacaattaatttggataatattttcggataaaatttttgggttcctacgtgtcaatactattcatatcggataagattttcggtttcaaatttcagataaatttcaaatttcagatacatttcaaaattcaaatttcagataaattcaaaatgtcaaatttcagatacatttcggaattcaaatttcagataaatttgaaatttcatttgaatttcaaatttcagataagattttcaccctctaagattgcgccgcgtgtcatatctatctgtgtacatttttctataaaatcagaggttcagctcatacctcccacaccagttcttctctacatttccatttgtcaaattttagatttcattctccattctcaatggcagacatggaagaggttttggagaggcaagagcgagaaactagagaaagaatgcgtagacgagctgcaagcaaaagggcgcagagagaactagatgagcaacttggcatagcagttgctttgctggaggaagaaaagcaggctcgccgtggttcacgagaaggccgtcgcccaaatgtggacagacatagacattcccggggtaagaatcttatggaagattattttatcccacaatctctgtactctgatgttcattttcgagggagatatagaatgcaaccccatttgttcaataaaatcatgcatgatatttgcaattatgatgaatattttgttcaaaagagaaattgtgctggaaatttgggacttcttccagagcagaaattcacagctgtgatacgaatgttggcgtatgggtcatctgctgatcaggtggatgagattgctcggatggggaagtccactattttggagagcttggtgcgattttgtgatgcagtggaaactctgtacaccagagactacctccgcaaacctacgcccagggacctgcaaaggcttctccaaaaagctgagtctcgaggattccctggcatgattggtagtattgactgcatgcactggcagtggaaaaattgtccaactgcttggcaaggggactacggaaatagaaaagggcagaaaagtatcatcctggaagcagttgcttgttttgatacatgggtttggcacgccttcttcggagttgccggatctcaaaacgatttgaatgtcctaggtcaatccccggtgttcaatgatgttttgagaggtgaagccccaaatatcacatatgaaattaacaataccatctaccagaccgggtattatctagctgatggcatatacccgaggtggacaacatttgtgaaaacaattccacatccccgatcccataaggaaaaattttttgcttgctatcaagaggggtacagaaaagatgttgagaggtgctttggtatccttcaagctcggtgggctattatcaggggcgcggcacgtctatttgacgaggaggtgcttaggagtataatgatgacttgtatcatcctccataacatgattgtggaagatgaatatgattacgatgctgatgacgtgtatgaaccaaatcccatggacacggccctaacacgaatttatgaaaaaccagtggggccaaatggagaagcagtgcagcatgaaccgttggttagagacggtagtttcatgcctcgtatgattgatcgctacacggagatgcaatcgtcgtatattcatgaacaccgtcaagttgacttgatggagcatttatgggcggtgaaaggcaatgaaggaaatgaaggtgaataaagtgaagtgaagaagttgtttttattttattatgtttatgttttatgctttggttgtgggttgtttattttttatgctttggttgtgggttgtttatgttttatgctttggttgtgggttgtttattttttatgctttggttgtggtttgttttttatgtatggaatgttttgaataaaaaggatttttgttgaatattttctttattgactaaaagaaaagcaatacaactaataataaaataaaatacatgaattaaacaaaacaaaaaatacaatgaaatgaaaggtacatgaattaaagaaaacaaaaaatacaatgaaatcaaaggtacatgaattaaagaaaacaaaaaatacaatgaaatcaaagccaagtaaactaagacatgaaaggcaaacaaactagtttaatggtttccatcatttaaccaatccgtgttgctaggtccatcgtcacgaaaaagtcttcgtctcataatatcccttcgttctagcttccaatattgttttgtttcaggggacatatggcttgtatccatggccatggtttcccgaccttttttttcaatgttttgttcgcgtacatactccctttctttgcgcacatactccctttcttttgcatattctacttgaatagccatatcatgctcttgttgtttcaagtccatttcaattctcatggcttggtgctttgaaagttcctccaaaaattgagatgcattcttgctagaattactccctctcttcgccttcgccgccttcctcccaataggtcttggctcattttcaattggtgagtctagattcatcggggaatccataggtgaatccgatgccggagtctcacgaagtggagtctcgttcaagactaccgtcggaccggttggaataatttggaatctcttacaagtcttcaccacctcccaacaatgggtatggatgaaactttttttcccttggccagtagcaccaaaccacatttgtgcttgtataatctataaaaaaaaatgaaatggaaatgcaagagaatttttaaaatattggcaatgcaacatgtaaaaaaaaaactaatggaaattaaagaattaataaaaaaatgcaacatgtattaaaaaaaaatagagacatattaacaaaatatataaattgcaagaaacatttaaataaaaattaatatgacatagaaattaatagaagaaaaatgacaaataatttacctcattgctaagattttctccgcttcgttggttgtcaattgcttttgctaaagcatttctccatttccccaactctttattaagaactttccacctactggataatgccatttccgtacgtgtagaaccaattgccctttcacaaaatgcttgatgaatttttttccacatatgagaaaatttaatctcattgcccgttactggacaatgactaatttggacccaagcctcacacaagctaacatcttccatcatgctccatgcccctccattttcattagaagaacccataatatgctagaaaaaaattacaacttgaaagtgaaaaaatattgaatagaaagtgaataaattttgaggagaaagttaacaatagtagaaggagaagaaaatatatgaggatttggtgttaaaagtgaagagtattggttggtatttatacacaaaaaattctgtaatttttgtgtatttttttaaaaaaaattcgattttttttcaattttttttggcagaaaaattggctgccgttggattgaagaaaaaattccaatcggagctaccagaggccgccacgtgtcaaagagccgttggcggcactgtagcgctgatgtgaaattttttttaacgttggcgcgtgcaatgcacgcgccaacggtaaaaaaaaattcgaaatttcagggctgacgccatgctggcgtcagctattttgtcctggacttcgggcgcgacttcgggccgatttcgccttcgggcctgcccgttttgctggggcccacgctcgcccgggctggacttttgctgctggaatcgattttttgcccaaaccccccccagcccgagtgtttccagccctgctggaaatgctctaagagcatttccaccagttgactcttgccatggcaagattagccctagggcaggcactattcatgtaaatagtggctgctctagtcccctccagcaaaatgtgtttccagcagttggggcttgccatggcaattactattcattttttttttttttcacaattttgtttacttaaacaattaatttggataatattttcggataagatttttgggttcctacgtgttaatactattcatatcggataagattttcggtttcaaatttcagataaatttcaaatttcagatacatttcaaaattcaaatttcagataaattcaaaatgtcaaatttcagatacatttctgaattcaaatttcagataaatttgaaatttgaaatttcatttgaatttcaaatttcagataagattttcaccctctaaggttgcgccgcgtgtcatatctatctgtgtacatttttctacaaaatcagaggttcagctcatacctcccacaccagttcttctctacattttcatttctcaaattttagatttcattctccattctcaatggcagacatggaagaggttttggagaggcaagagcgagaaactagagaaagaatgcgtagacgagctgcaagcaaaagggcgcagagagaactagatgagcaacttggcatagcagttgctttgctagaggaagaaaagcaggctcgccgtggttcacgagaaggccgtcgcccaaatgtggacagacatagacattcccggggtaagaatcttatggaagattattttatcccacaatctctgtgctctgatgttcattttcgagggagatatagaatgcaaccccatttgttcaataaaatcatgcatgatatttgcaattatgatgaatattttgttcaaaagagaaattgtgctggaaatttgggacttcttccagagcagaaattcacagctgtgatacgaatgttggcgtatgggtcatctgctgatcaggtggatgagattgctcggatggggaagtccactattttggagagcttggtgcgattttgtgatgcagtggaaactctgtacaccagagactacctccgcanacctacgcccaaggacctgcaaaggcttctccaaaaagctgagtctcgaggattccctggcatgattggtagcattgactgcatgcactggcagtggaaaaattgtccaactgcttggcaaagggactacggaaatagaaaagggcagaaaagtatcatcctggaagcagttgctggttttgatacatgggtttggcacgccttcttcggagttgccggatctcaaaacgatttgaatgtcctaggtcaatccccggtgttcaatgatgttttgagaggtgaagccccaaatatcacatatgaaattaacaataccatctaccagaccgggtattatctagctgatggcatatacccgaggtggacaacatttgtgaaaacaattccacatcctcgatcccataaggaaaaattttttgctngctatcaagaggggtacagaaaagatgttgagaggtgctttggtatccttcaagctcggtgggctattatcaggggcgcggcacgtctatttgacgaggaggtgcttaggagtataatgatgacttgtatcatcctccataacatgattgtggaagatgaatatgattacgatgctgatgacgtgtatgaaccaaatcccatggacacggccctaacacgaatttatgaaaaaccagtggggccaaatggagaagcagtgcagcatgaaccgttggttagagacggtagtttcatgcctcgtatgattgatcgctacacggagatgcaatcgtcgtatattcatgaacaccgtcaagttgacttgatggagcatttatgggcggtgaaaggcaatgaaggaaatgaaggtgaataaagtgaagtgaagaagttgtttttagagtaggatgtttatgttttatgctttggttgtgggttgtttattttttatgctttggttgtgggttgtttatgttttatgctttggttgtgggttgtttattttttatgctttggttgtggtttgttttttatgtatggaatgttttgaataaaaaggatttttgttgaatattttctttattgactaaaagaaaagcaatacaactaataataaaataaaatacatgaattaaacaaaacaaaaaatacaatgaaatgaaaggtacatgaattaaagaaaacaaaaaatacaatgaaatcaaaggtacatgaattaaagaaaacaaaaaatacaatgaaatcaaagccaagtaaactaagacatgaaaggcaaacaaactagtttaatggtttccatcatttaaccaatccgtgttgctaggtccatcgtcacgaaaaagtcttcgtctcataacatcccttcgttctagcttccaatattgttttgtttcaggggacatatggcttgtatccatggccatggtttcccgaccttttttttcaatgttttgttcgcgtacatactccctttctttgtgcacatactccctttcttttgcatattctacttgaatagccatatcatgctcttgttgtttcaagtccatttcaattctcatggcttggtgctttgaaagtcctccaaaaattgagatgcattcttgctagaattactccctctcttcgccttngccgccttcctcccaataggncttggctcattttcaattggtgagtctagattcatcggggaatccataggtgaatccgatgccggagtctcacgaagtggagtctcgttcaagactaccgtcggaccggttggaataatttggaatctcttacaagtctttaccacctcccaacaatgggtatggttgaaactttttttcccttggctagtagcaccaaaccacatttgtgcttgtataatctataaaaaaaaatgaaatggaaatgcaagagaatttttaaaatattggcaatgcaacatgtaaaaaaaaactaatggaaattaaagaaataataaaaaaaatacaacatgtattaaaaaaaactagagacatattaacaaaatatataaattgcaagaaacatttaaataaaaattaatatgacatagaaattaatagaagaaaaatgacaaataatttacctcattgctaagattttctccgcttcgatggttgtcaatcgcttttgctaaggcatttctctatttccccaactctttattaagaactttccacctattggataatgccatttctgtacgtgtagaaccaattgccctttcacaaaatgcttgatgaatttttttccacatatgagaaaatttaatctcattgcccgttacgggacaatgactaacttggacccaagcctcacacaagctaacatcttccatcatgctccatgcccctccattttcattagaagaacccataatatgctagaaaaaaaattacaacttcaaagtgaaaaaatattgaatagaaagtgaataaaatttgaggagaaagtgaacaatagtagaaggagaagaaaatatatgatgagttggtgttaaaagtgaagagtattggttggtatttatacacaaaaaattctgtaattttttgtgtattttttttaaaaaaattcaattttttttggcagaaaaattggctgccgttggattgaagaaaaaattccaatcggagcgaccagaggccgccacgtgtcaaagagccgttggcgacactgtagcgctgatgtgaatttttttttaacgttggcgcgtgcaatgcacgcgccaacggtaaaaaaaaattcaaaatttcagggctgacgccatgctggcgtcagctattttgtcccagccttcgggcccgagctcgggccgaattcgccttcgggcctgcccgttttggtggcccccactctcgcccgggctggacttttgctgctggactccgttttttgcccaaacccccccagtccgagtttttccagccctgctggaaatgctctaagataagaggatcatatgcctgcctgcaaacgttgttggaaatttccctcacccgtgaaaacgaatttcacgtttttgtttttttccatttgggaggacaaaaggtgttttttattattataaataaataaagaaagagaaacttcgttaatttgtaaccagaacaactggaaagaaaaaaaggggtgtacggtccctgaggttgtctcatcaaaacctcgtcataaaaagccccaaaacaagaaaatcccgtgaaggaaaaagcgtaccacacaaccctatgtttgttgtttgtatttttaaaaaaaactgcattcctccctcatgtcaaaatgtccgcaaaaatccttaaaccaaggattatcaggacatataaagacaatgacatgatccaaactcaaagataatcatacataatacatataaaaaaaataaaaaatcgtatgagtacagcagttgggcgtctaagttccagttctgcaataatttggtgcccaggaataggacctgtaaatttgcatgcacctactttttcttggttcacaaaattgatcaacttctcctcataaagtcaatcaaatttgatgccagttaaccatccatcctaattcatttcatcccatttgcgtacgcctcagcaatgaagaatttgcaaaaggatcactttcacctttaaatgcgaaaaggaccatccacacaccaaagtacaatgttttcctacgttaagttgaaatgggaattttctctaagcatgtggtcccttgtaaaagtactggcatttgtagggtacaccattaatccaaaaccaaatctatcatgtcgtacagccccttttttttttttttttttttttttgggcagtagtatgtttcctaaaatcgcagcaattgctctgagaagtgttacaaggaaattagtttgactcagaacaaaaacaataacaatgatgttaccaacaaaatactcagtctaaggaaaggcagattaacagcaaatccattctaaatttctttaaattcctccccaaagggaaaaccaagaatgtttatgtatctccacaaatcaccggaaaaacttatagccttaaaggtagtattcaccctttatagttaggggttgcgggttaagcctattagacgttcaaagtcgaaccgcacttacaatgaccgcacagaatactcccatacattatttctatccagtatacttcatcatctacatcaaaaactgctgctgacactcatttataacaggggatgctgtaacagatgtgatgcagcagctgcaactatagaagtaatgacatgaagtttcgtgtgcacgattcttttagagttccaccgaaacagaaaatatgaatatgaaaaaacaattagttaggctcgttagaatcagttaggatttctatctgaagcctgaatgcaaggtaatgagtttatatctaaccagtacttaaacttcatggagtctctaccctctgctctatagcctcggttaatttaattttagtcttcttgtttgtctgtcaacctctgcattcttttacatattgaacatcacacagtaactaccatagcaaaagaacaatcacaatttttttcttcaatacgatatcatagaactcgagtgttaaaaaaaaagaaaaaagatttcagttctgttcagttctgttcagttctgttcatctacacccaagatgattcagctaatcagctaccttgatcagacatgcacccacaaaatttacatgcgcatgcatccagtataaagaggcagcaaaaggctctatagaatggttgaaaaagcttaatggcatcacattactgattacaaatcaactacaaaatctacaaacgcagtcagttcaataacttcagaagacaacaacatctacaaagcaacaattacattctaccccaagatcaacaacaacaaaagtaaagactgtaaagagagtaccttccgcgttttccaaaccgagcagcttcagaaaaccaacctaaaaattacaaagagtcatctgccttaaaccaccaaatcaaataaccttcctgggccgtcccctcttcttctgattagtccccaaatccgggttcccatccgaaacatccatcactccacccgaattggccaccacggggaccgccgtggcagccaccggagttttactctgcggcggcgtcgtcgaaaagacaggtggcatccaattgggaaaggcggctgcggccgctgccggggccgccttacccttcccgccattggagtgggtcccgcccacaaatcccatcggaaaaaacgcccaacagcagtagtaggcctcctggccttgcaccaatggcggcaaattcggcacaaccaccgcctcaaacccgcgcttacaattctggcacctcaaacagcaattctcgtaaaccctagggtactcgtacagcacatagcagtacgggcacgtcgtccagaaagtcgacaatctcgacctctgctgctgatgatgctcaccgtcgtttgtcaaatcggcgtcgtttcggctccgattgactcgccgtacaggcaatttgttcgaattcggtgcgctgagatcgactcggctgaacgggcccaactcgttgtcgtaaatcggcttccgagtcgggtcggacaaaacggcccatgcatcggcgacaagcttgaacgcgtgctcggcataagcgtacttgttcttgtccgggtggaggagcaacgccagccgtcgatatgacctcttgatgagatcctgatcttcggatcggcgatcgacctggagaacggcgtaccagtcgtggtggttgtttacgcgcttgtcggcggccaagagcacgtcggcgacggccaagatctgatctgagccttccaatagcggctcggtctcttgcgccagaattgcgaagtcacggcagctgctcagatctcggctgtgtagaagcttctcggcgattccgagcaagcgctcggcttccactctgtttggatccatggatttggatggccgatagcgtttgctgttctggttttctgggaaaacaaaaaatgtcgaaacgcaaaatggaaattgaaatcgcaaagttcctctttcactcagtctcttttttttcttcgaagatatttattggggcttgattttgcttttaattgcagagtgcttcagaggtgatcttatttttggtcggggttgttttggtcattttaatgaacatccccctgatcatggccttgacccttcgcacggttttcgaatcggaatcggaaatttggaaatggtaatggcgcaaaaccaagtgcccaagcgtgccaagagatttcattgggaaattttttgaaaaatcctctcgtatacgtttcttttttgggttatgctagggaggtcaactttaaatactaatttgtgtaccaactctttaatagaggtggaacccaccaatacaatgggtcacacactctattagagagttggtacacaagttagtatctaaagttggtctccctagtattttccttatttttttgtgtcaaatctcacatacctctttttttactctaccaaaattatcgaaaaggaaaattactgtagtaaatcaatatcaactgcattgtctatcgcattgttagtcactccttcatatttgttacattttatgttaaatttagaaacaaatgataattttaagacataccagtagtttttaggagcaatagtccttgaacttttagtaaatttacattttgattctccaactcgaatattgacaccacgaattcatctacttcactttcaactcaaatattgggagcaatggtccatgttatgtagttttttgttttcccaccatcttccttcaaagtcattgcaagccatcattccctctccttacctgtaatgattttgtgtgcgtgtgtcagatttctatgatagaaagggatcgactaaccttgctagacttaacatggaagattttgagcagtgcaacgtcaccaatccatctagtacatggtttggacgtgatcattgcgataaaggacaaaaattagcaatttgtgttagaaatcctttacatacttgatctcctctaaaaagcaaaattcctttagactcaaatcacaggttataaattaaatcccaaccaataagaatttgccatgtatcacattgtttgatatttaaaaagtcaactggagacatattaatgaagagtttcatgcagtagaaagataataatgaagtgtttcaggttgttccatccacaacaaactgtatagatcaagttttttttgtaacttttttcattattaatgtggaggcattgttgatttcatatggtcgcatgaccactcattctttatgatcaaatagtaacttgtttacacaatcttatgttttcttcatgtagcacttgcttacatccagtttgaaaggaggtgaggggggcccgagaccacacttttcccttggtggttccgctcttgattatggtgatgattttgttgattactgtgaaaatgcatgcaagacaataaaggaaaatagaaacttaatatcttcaaaaacaagttaggctccatttagattgagagattccaaatctatgaattcatatgatgtgtaatttcactagtaaaaaatgtaagtgggcaaaatcaaagatcactggacaaaattgccctcaagcacagtttgcacacgcatgaaaaaggatccacacttaattacacatttattaggagcgaatgacatagtttcaagcatctagtagaggaagtcacttatgctagttggctagagcggatgtgctactcactctgcatttgaattcgaatcgcccttagtttaaattagattaaagtaaaatatcgtttgtaccaaaaacaaaaaaaacaaaaaaaaacaaaaaacaaaaaaaacaaagcttatcgctcctccatgctagcaatggtattttgtcaacctaagtggccactcagtccatccctatgcaattctcgaatcagtttctcccttaaagaggaaataggatgcacaaacggttgcctttaaataaaaatctttcattaatctgataatttttcatcggctgtttgcgaacacacttagcccaaatttcttgaaaatcttagtcaaccttatagagttctttaagaaattcaacacccacaacttctggagcgaaagaaacgagtaaggtggctcgctaactcaatgcatctgctatacggttaagaattcaagacttttcttattgacaaagttctgtagaaaagcggccaacatgacatgcatcttattgacacttttctagttgttaataaatttcaggacttggtggtcagtgtaaagcacaaattcccattgcactaaataatgcttccactgcttcaaagatcgacccataacataaaactcctgatcatatgtaacatcccacatcgaccaacggagagggggtgatgtgccttatatgtacatgtccacctccatctagcacgaggccttttgggagctcactggctttggagtccatggaaactcagaagttaagcgagttggggctagagcaatcccagaatgagtgacttactgggaagttgcttgtgagttcctaaaaacaaaaccgtgagggcagagaggggagcccaaagcggacaatatcatgctacgacggagccgatccaggatgtgacatcataagtgctccatttttgacgtgcttcattgagtttttctgaaggaaaaaaaatgcaaccaatctattctctttagtagaaaacaactcccacacccacttcactcacatcacattcttggaataaacttttcaactagaacggggtggtgctaagcttttctttaattatggtaaagctctgctcttggtcctctccccgattaaatttttcctgcttcagacattcagtgataggtgcagtaataatgctggaatttctgacaaacctcttataaaacgttgcaaggccatgaaaactccgcactcgctaactgttttcggggtaggccagtcccgaatagcttgcacattgtcttcatccatataaatgtcatatgcattaacaacatacatagaaaaagcaacttattggtgaggaaagtgcactttttcaagttaacgtataacttgttctcctgcaaaacttgtaaaaccttccgcaaatgtaccatacatcatgacaaagagatgaattttactcgtgacgtcattctctttgaaacgacagatcatggacctggttccggactcaaggcccaaacctatagttttctagctttgccttcacgcacacgacgctcctgctcttcctcgatcgctatagccatcagttctatatcacataggaagatgaaggatagcttaatagagttagcatttagttaaggtgagacgacaaatgtcagaaggataataagaagacatgaagggtacatacaaaatgggcacatgagaattgaacttggtgtggactgtggccctacagtagatgagccattaagggaccagtgtggtcttaggcccccactctatttttttctaattatatatatatgatatatgatattgtaatatattagcattagtttgaggatattcatcctaaaataaacaatatattaggttctcattgtctacaaattaaccataaaacttcgtgatctctcatctacttgtctaccttaaattttttaggttagtttatgatatttgtactaaaaactcctaaatttacaacatttctttctttttatcttttgtttgacttaaactttttaaattttttatttccgtccttttttcatgtataagttcattgagttgggaatcttagtgttgtgatttatgaacttttttttaaaattcttttctatgttggaatataaattattaagtccagtctttaataatgttgacgaatttaaaatatgaaatttatgtttctaaaaataaataaaaaattcattacaattatgattgtaacgaatatatattattaatttatatcaaatgatattatgaaatggaccaaaataagtagttagtgtcgaattaacttattaaacggttagcgttctaacacttttattcaaataaaaatttataattttataaaggccttttttaatatgaaatcttgactatgccactgtatcatgtgaagtaacccgagcaaagtaagaagtgtcgaatggtgagttcaatcaagtgagacgacagcgcatcacctgtatcaaggaccatcctcctccacataaaagcacaatttttaatatatttttttgaagatactaaacttagatagtgacaaaaccattcacaaaagtaagatgcaacattttttacctgtcacattacaaaatcaacggtggagatcaattaattgaaatatatatatatttttttcccataaaaggaagattgcctttgaattattctctttcttgaacagccagccaccaccagcagcggcagtcaactctctctctctctcctctctctcattctcttaaattttctggcttcagaccctgaaactccccgacaaaggtaaacattaaacaaagagaggaaacccagaatttaattagttaattaattaatggttttccctctttctcttcttcttcttctcctttgtgctctggtattctctaggcatgttgtgaacttgtgacctatattggttggtgggtttttctgggttttattcccttagatctgggggccattaaatctttaaaatttacaagaaacccagaaaatattgtgcagtttttgagtgtatgaacataatatatgtgaaaagtggttggtttgaatttttttgaaggggacgatgaaaatgatgagggagaagatcaagatcaagaagattgacaacttgcctgcaaggcaagtgaccttctcaaagaggaggagagggatcttcaagaaagctgcagagttatctgttctgtgtgaatcggaggtggcagttgtcatcttttctgctactggcaagctttttgattattcaagctcaaggtaccagcaacacttctttgacttttccttccttgaaaaaaataaccagttaggtatgatcatgaatcatgatgtctctgtactctgacttgacttctctctgtgtaagtgagagagggagagaagggtggaggagaagagtgtcttcatggaaatattggttctttcctgaaattccatggtttgtttttttggtcgaaaatccatgtaaagaaaaatacaatgcagaatccgaaagcttctaaaaaggggggagagagagagagagagagagagagagagagagagctagatagttgtactgtatttcaaaggaactagttcgacgattactacctaccatatacttaaggaaagtaaagtctacttttgattaagacaacttctatgaaacattattttattagttttccatgttttcatccttgctgcttcaatctcatcatattaggtggggcttatacagtgaaaaatatccttgtccccgtccaaggagttaaaactaatggtttccctgtgagagacacagtacgatagattatcaattcaatcatacacttaaataaaaatattagacacagtatgttagattatcaattcaattatacactcaaataagaatgttagacacagtattcccacaaacggcaaactttatactagattcatagtcacaccatataaaatgatatataatattccttcaaacttaagatagagggggggtcgtgtttgaaaaggcgtcctttctgtctcctcacctaaatgtggttacaaacattgaattatatgatttgtagttttcttactaggaataagttgatatgatgataactggggaattcatcataaagccttgaagactttcagcaggaatatgcacccattttctcaagtagggcaatgggtggtggagctattaccataacatgtttttgccatgaatgcatgcatgtgaggaaggaaaaagagggaggagggtgagaatgaaatgttaactgtggaataagaatttaaatatggtcatgcttgctttcggtcaacaaaaataattataatttatatgtgcatgcatgagatgagaggaagaaaaaaaaaacagggaggatgcgctgatcatttatgtttttttttttttggtttaaaaaaaaaaaaactagaaataccacctctaaggtggtcttattttacttttctcgaagtacaatttgaaaatgcaaattaagaacaaaattagatgacaaagaaaacaaaacacgcgtactaatggcctttttgatacacaagtggaggtctaatcatttgtaattatgaaatttctaatcaaattccattgaaaatttcatttaaaagttaattttctaggattccagaacctattagtttgtttacttgttttattttctattttttggagcaaaaacaaaaactataaaaacaaaaacaaaaactataaaaacgattttactgaaatcaacatacaattaaaactaaagaagaataagtgtgttgttctttagccttagattttattttaaattttttttcaaacttctccaagttccaagtagagcaatattaaaacgtttggaaaagtattgctctacttggttctattatactgagttttcgaaatatataatgtgccgaaagtcatcgaaaaaactatgatatgctaaaacaaatgattccttatgtagttaagtttcgagatgttatatttatatttgtatgtttgaaactctaagggcttgatatgcgaggacatcttaacacaagatatagcatatgttgtctattcttactcacatcaaattgtatatattaacgtgccaagcgtccttagccacttccactaagtttttttcccctaggagatcattcctcttcccattttagtaggtttcaccacttccattgcatccgtctaaatagtccgggatgtaaatttaggttgtagggtttttttaagggcatgtttctctaaaaccactagagagttgtcacgacccaacttcactagtaagatattgtccgctttgggtttgggcaatcatgtttttgttcatgtactcaaacttatgctatttttcccacaagacatcttggtaggaagtcaagtgcccacgcatatatggcacttcaattttccatccccgaggaatgtgagattgctcactgatcttgcccttcttgccttgcatccttcatggatcacaggtcttacaatccacccctcttggggctcagcgtcttcgctggtacactttcagtcggggtccagctctgataccattctatcatgaccctgcttcactagcaatatactggccgatttgggcgtaggccctcacatttttgttcataagatcaaacttatgctatcttacccaaaaacctcttgataggatgtcaagtgccacacgtatatgacacttcattttcctctccctgagggatgagggattgctcactgctcttacacattcttgtcttgaatcattcagggcttacaagtcttacaagagcgcccaacgtttgcttttgtcaaggttcggttgccaaactatataatagtttactaaacaactacttataggcccttccacttgtcacttttgaaatactaaggtcgggtttcgtccctgctcgagtgggtcttgtagtagctatcttcttccattgcactaaacggtaaggatattggcatggcattatgtcacttggttgtttcttaggatatggctttgacagtgtggcctaattgattccggaacttacaaaatattttgtacatttaactcgtcattgtttaatagtgaataacgaatgttattgactgttgttgcgccctaggttagaattacacaatacgagaaccataaacatacggcttttcctccttctccacttgacacacttcgatcctaagaatgtttggttttaagaatgagtggttagccttctagaagtatcggttttctcctaacatgtcttaatatgtccattagaatgagtagccaaagttggacttgatacatcgaaaaagtattggtaacttcaatcacaaagttacgaattattacgctagtaattccttttatctgtctaaacctaacttatattctaactctaacttagttgctataaggagagatgagaatagctcaagtggcttcaaatttgtattcaagtaggcaaaaaatttgggcgtagagagtgccacttaaaaagaattgcgattagcttgacatactgacggggccaatggtgtaacagtttgtgacgatacagttatttttgttatctctagtattactggcgtcatatcacaagggtgcaaaatgcatttgaccctggatatttttccaatacgtttggcatatatggcctacatagattcagccatccactgactaaaaaggaggcgaaccaaagataacaagattcaaaacatattgcaatgagatccagagttgatgcgagaggaattttacttgacgttttgatttgcgtaaccatgcagccagccagccagccatgcatgcagccagcaaatagcttaacgtacgttctcattgtgcatcagttttgggccttttggctgattttacttgcaacacgaccacatctcaaataaacgtgttcatttgatttcaaaatcttaaaaatcttacttgtgatgatggaatgaaatcttgattgatgatgggggtcagattatttttttccttgggtaaattagttcctctgtttttcactgtttgttgatgttttttttttttttggtaatgctctcagtgagcggaggtagagaattttagtattaaaaaaattacagattattaaaactctgagtattccttaaaattggtttcttattgaaatagtgcttcttaaaaggaagcataccatacaatagtatatacaaaagaaaatagacagaagtgtgcctccgtgtttgtaaaccagaatttttcataaggagttaattattttccttctttttttggtttctcatgtatgttttgtcacatgttttaatttgatcttctatgtctactaacttacaccagtgaacatgccaattgattgtgttttccttttgtgttgtgaagtatgaaggatgttattgaaaggtaccaagagcacataaatggtgctgaaaaatttgacgagccgtctattgagttgcaggtaaatgagtttaatggtttaccttttccatttcggacaatacttccctctttatctataaggaggaactccaccttttaccttatattttttaattgtcaaaactgctttttttttttcctttcactcttaattaaaaaagagaggacacatatcattttgtgattaagccacaatagaagcgaagagttcctccttacataaggaggaaagttttgtccttccatttccccgctttaaattgttgtgcaattaattataacatattctgtgaactgcactttggaccttcaaaatgcgtttctatagcatatatgaaggttctgattgtttgaacataagacaaaaacaccattttgttcgttacactaaggctcgtttgggactacttctttaaaaagtacgtttgctcataagtgctttcactagaaaaaaaaaaaagtggcaatagtcaccattatttggtgacatttgtgattttagtcaccccaacattagtcaccaaataatggtggctgttgccaatttttttttttttgggtagtgttctagcaaaaatgcttttattataaagatgttgaaattttcattaaaaaaaaagtgctttctagaaacgcactttggattgcttttataaaaaattacgcttttagcccttccagaagcactaaacaaaccttagacgtatgtatatatgcatgcagcaggtcgtcctcttgggctaaatatatgtaggtggatttgacaagtccatttgtagtgggacttaatttggtctctacataagaattagccaatgaaaaaattggaacatagacatcaactcagtcaatcagatttttatagtttactcttactgttagttgtgttccaactgttttgtttttattttttaattatacacaaggaagatagtaaattcttttaatttttaattattggtgacaaactggcttatgttcttgtatgtttaagtaattaatttgattccattttcaaaatagaaaaattgtaatccatttttttataagcttttcaagtgacagacagattagatttacctaaatgatggtttacgtaattcatttgattccattttcaaaataaagaaatcgtatgtttaagttgtttccttttctggactgatgtttataagcttttcatgtgacagagacagattagattacctaaatgacggttccatttctcattttagccagagaaagaaaaccacatcagattgagcaaggaacttgaggagaagagccgccagctgaggtaataattatttcattcaggaacttgtaagatatatgtggctgtttagctggtattatatcgtttgatgtatctgctgaaattggactggcttgcaacaggcagatgaaaggagaggatcttgaagagctgaattttgatgagttgcagaagttagaacaactggtggatgcaagccttggccgggtgattgaaactaaggtctgccctagctaatttcttttctattaagcatggtgcgaaggcatcactcagtgttgtggtttagggctcatttgagagtgattctggatgcgctagaatcacatctgggaaaaagcatattccatgtgcttctccatataatcacttttagtgattttaagtgattctggggagaagcacctcttatgtgtttcttcttaaaaacacttaaaatcactaacagtgattctccataaaagtgattattgaactatccagaatcacttgcaaacgagcccctaatgattgaaaccgttgaatggtttaatgatcgaaaccgttgcaaatgttccattcctttcaggacgaactgattatgagtgagattatggcacttaaaagaaaggttagacgattaatacgtactctgtaaatgagaaacaaatttcaactatttacaataatcaggatttttaaaaaaatatataaatttaacatcaataatcataaccaagtttggctctggttttctcttccatgcaccgaatctaatttcagagaagctgagcttgtagaagcccaacaaacagcttaaggcagagggcaagcaactaccacaatcatgtacatattttcttcaggattcttctttctgtttgtttttaaatgaaaaaaatgggtgatttttttcttaactttgtattttttttgcatagcagatgctatccagaggaaatattggacctgcgcttatggagccgggagaggttgaataataatattggtggtggaggagaagaagaaggcatgtcatctgaatctgctacctccaccacctgcaacagtgctcccagtctctctcttgaagatgactccgacgacgtcactttatctctcaaactggggtgagctagctacttttgctttgctatattttgataattatatgttttatatatatatatacacacacacatatatgttcatcaaaattttacatttttatttttatttttgttattgaaaataatagatggcttattagaattatcgtgagcattattgcgtattatggtggttcaaataaaattaaaattaaaattaaaattaagttgcccgcattgttctatcgaaaatacagaaactttcggttgcaggatttcaacacaaaaggggctattgaaaacatacagggaggaaatgtattccactctctctagactaaaacaacataacctgcataaagtcatatacwagttaaaatatcttcaccaacaaatgttgatgttgatgggcagttgcattcatggtatttcaattactcttcacctttttaataaatttgcttatcactttttattatctctgaatttcaggcttccataatttctctggttaaaaagtggagggttgtgattgtgtgtaagataaataaagaaaatcgagatattgtaagatatcagtgattgtgtgtgtcttaactcgatacaagattgaaaaatattgaggggaaagtaacttgtgttgtatctaaacctatgtattttatgcaacctgacatattgctctttgacttgtattgtctttagaactctgtatcaatgcagcctgctttggagactcttgtggactttgtatttgcttgggttatcaatgtaaattggagcatgtctcatagagcttatatatgaaataattgtccaatttaatgagttcaattttgttttttatttagcccagaatgtggggaaaaagacaactcattgactacaaaaaagaaatgatgtagaacagatgacggaaacaatcgaaaatgagttgaacgagactcacgcgcaaggataaatcgaacaaactgcatatttgggctccgaattcaaaatcaggcccatgattcgttgattcgaatagaacgacgtcatgagtaaagcttatttcttagccatgttgtatgggctttttcgggcacccgagattgtttagaaattcgaaatctagttttaaagtttttacaatttttagaaacttttttatttttttattttaattccttgattaatcctaaggtttctaggagtatttaaacaaattttacctgtgactagagttatgttttattatttcaattatcaaataaatcttgaaatttcctataaggttgtatatgcttggcccattggccctttgagtagggcctgggcttagtttattgaagatagggctgacccgattctgtcaactttatttgcgcatgaaaaaaacacccatatataatcgcattgtctgcgtggattaaattcatacaaaaaagaggagtgggctggaggagcaaagacaaagccaagtgttgcgactcgaatggtgcataacccgagcaaagtaagctgacaagacagcgcaggacctgtttcaaaggccatcctcctccacataaaaatgaaattcccttctttttttctttcgcagcctgacaagtactaagaattaaacccactccaataaaaaccaaaaaaaaaaaaagaattaaacccactccaacagaaccattcacaaaagtaagtaagatgccacattttctactgtcacgatacaaaatcaacggtggagatcgcatttatcaacttcaagtgcatctgtcaccgtcgaattgctcaggtttcgtacctagttttcccaaaagataccggaagtatgaaaaaaaacagagaataatagaaaaaggtatgtaattgtttgtccttaaaagggaaaaattcagtttgaaattcattttaatcccttccttcgttaaacagtcagccagcaggagaagcagccatttctctctcccattctcttagtttttctgagaacaccaaaacttcagaccctgaaaccccccaaaaaggtaaacattaaacaaagagaggaaaacccagaattttaattagttaattaatggtttttcctctttttcctcctcctcttcttcttcttcttcttcttcttcttcttctttgtgctttggtactctaggcatgttgctgtgaacttgtgacctattttggttggtgggtttttctgggttttttttttttcacttagatctgggggccattaaatgtttaaaatttacaagaaacccaggaaatgttgtgtagttttttagtgtatgaacataataatgtgaaaagtggttggtttgaatttttttgaaggggggatggtgaagacgatgaggaagaagatcaagatcaagaagattgactacttgcctgcaaggcaggtgaccttctcaaagaggaggagagggatcttcaagaaagctgaggagctatctgttctgtgtgaatctgaggtggcagtagtcatcttttctgctactggcaagctttttgattattcaagctcaaggtaccagcaacatacgttataattcttttgacttttccttccttgaaaaaataaccaatatgacttgtcttctctctgtatgggtgagagagggagagagagattctaagattcagtcatttgggtcaaaatatatttggaggagaagggtgcctttatggaaatattttttctttcctgaaattccatggtttgatatgaactctggtctagaatgcaaagaaagaaaaaaaggagaatccaaaagcttctaaaaaggagagagagagagagagagagagcggtagatattgttgtactctttgcaaaggaactagttcaacgtttactacattatctatgtacttcaggaaagtgaagtctacttttaattaagacaacgtctaggtgggtgtcccaatttgagaaatattattttattaggtgtcccagtttgagaaatagtattttattaggaaatggataaaattaatggtttcctggtgggaaacacggtatgttagaccatcaatcaaatcatgcactcaaacaagaatgtagatagtcaattctatctgcgtaatctgacaacataaaatgaaagatagtgaagagagtattatatgattcgagtattatatgatttgtagttttcttactagggataaagtgatgtgatgatgactttgggaattcatcatagaagccttgaagactttcagcaggaatatgattccatgctctcaagtagttctccccaagtagggcaatgggtggtggagccatgatcataacatgtttttgccatgaatgcatgcatgagaggaaggaaaaagagggaggagggtgagaatgaaatgttaactatggaataagaatttaaatatggtcacgcttcctttcagttaacgaaatataattgcacatattcagcactgatcatttgtattaaaaaatgtttttaaaaaaaaaaaaaaaaaactagaaatatcacctcactaaggtggtcttctttaacacaaaatacactgcataaaaaggtactctctatatgtcgatggttttttaagttttctatggtgtggttatgaattccgttatttagatgtttttattttgatttcttttgttaggatgagagagtgggagtattatattgggttgcctcaagattgtctcctacgtcaccattagacatgcgtctaatgaaggaaagagattaaaaggaggcacacatatctcgaactacaatttgaaaatgcaaatgcaaattaagaagaaaattattaaaagacaagggaagcaaaactataagtggcaaacacatactaatggcctgttttgataataaaaaatgatttgatacaaatggaggtctcagttaaaaattacaaaatggaagagaccagggcgaaattacctacaaggatatcctgggctgtagtccagcctaggtagctttttaaagatattttttttaaacctgttgtagtccatgtttaaagaaaaacaaaagtaaacctatatagcccatccacttgtcgttcccacttgtggttggggtcctcattttgttgtctttttgacaattttcccactatttctatgaatatggaaatattgttttttcaaagtctgttttgtggtcttcttgataattttcccattgttatcatgttaacccttttgttgcatttaatttgtgacccctattggtagtataattggatcaggtcggagcattggcctcaagtacatatccaagagtatcttaattggttagagtctaccatttactccctcatgcaaggttttgggttcgagtcctagcattcgtgtagtgtgtgtgagtttcgtatgctatcgcccctctcaataggaaaagttctcaaaaaaaaaaaaagttttattcatatttttatttttgaaactctaagggcttggttagcaaggccatgataacacaacatatagcatatgttgtctattcttaatcatagcaaattgtatatactaacattcttaagaaccttagccacttccactaacaagattcaaagcatattgcaatgaggcccacttcttattatttgacttgatatggtctcaggtcggtttgccttgtgttctaaaatatttcagctgcttggtcttatatataataattctataaaatagatattaaggaattcaatcaaaagcaagatgtcgtgccatgctttatggcttagcgctgaagttttgtcagtgagagaaggtagagaattttagtaattttattgaaatactccttaaaattggtctcttcttgaaatagtgattttattttaatagaaacgatagtctaaattagtctagattaatctaatttatggggaggggggttcgaacttgggtgcaaggtgggtgggctcagctcactgtcctggccaaccaacctaaccaacgtcagctgaaatagtgttttttttaaaaaagaagcataacatacaatagtctatacaaaagaaaataaacatttgtgttggcaaaccataaaacatgtatgcttcgtcacatgtgtcaatttgatcttctatgtctactaacttaaacttgtgaaaatgccaattggttgtttattccttttgtgttgtgaagtacaaaggatgttgttgaaaggtatcaagcgcacacaaatggtgtcgaaaaatcggacgaaccgtctgttgagctgcaggtaaatgagttcaatggtttaccttttccatttcccccctttaatttttctgcaattaattgtaacatattctgtgaaatgcactttggagcttcaaaatgtggctctatatcatctgaggttctgattgtaaaaccttttggtttgaaaaagtgaaaaagggaaatttaacaagtatgtcctacgtcacatcacatgttgtgcgtctctctcagtgttgctgagatccaaaccttgaaacttgtttaatctctccacaaagggtgccccaaacctctaggcggtgacaaactctcactttgttggttgcatttgaggcttatgttcttgtatgtttaagtaattaatttgattccattttcaaaaataaaaaaataaaaaaataaattgtaatccactttttataagcttttcatgtgatagacagattagattacctaaatgatggttccatttctcattttagctagaaattgaaaaccacatcagattgaccaaggaactggaggagaagagccgccagctgaggtaataatttgtaaggtatatgtggctgtttagctggtattatatcgtttgatgtatctgctgaaattggagtggcttgcaacaggcagataaaaggggaggatcttgaagagctgaattttgatgagttgcagaagttagaacaactggtggacgcaagccttggccgtgtgattgaaactgaggtctgcgctagctaatttcttttctagtaagcatggtgcgaaggcattaattactctgtattgtggtttaatgatcgatcgaatccgttggagaaagttagttaattggtttaagacattgaatttgtgttccaataaataaacaacacaaggtcctaaacaaacactgaaatcattaccatttaatgaaatggtcaaatatagtctctgatattgtttttttttcttctttctctcgatgattcctgaatgaaaacctaaataatgaagcgttggaacaatataaagtatgtcccacaattgtcctttgtgcaaacagggtatataattgttctttttattgaaaagcataggtgtattctgggttgtcactgaggttttgagttgctattttagcagctggtttattctctgaaaactataggtgcatgcagtttgggttacatatatgcaaataagctataagatgtaaatgcaaatattccattccttttcaggaagaactgattatgagtgagattatggcacttgaaagaaaggttagacgattgatatacatactctgtaaatgaaaacaaatttcaactatttacaataatcaggactttaacatcaataatcataaccaagtttggctcttgttttctcttccattcactgacattaatttcagggagctgagctggtagaagccaacaaccagctacggcagagggtaagcaactgccacaatcatgtacatgtttcttctttcttctttcttttctttttattttgtattttttgcatagcagatggtgatgttatccagaggaaatattggacctgcgcctacggagccggagaggttcgttaataatattggaggtggaggagaagaaggcatgtcatctgaatctgccacaaatgcaaccatcagcagctgcagcagtggtcccagtctctctcttgaagatgactgctccgacgtcactttagctctcaaactggggtgagctagctacttttgctttgctttgctagatatttgctatttatatatatagtttattgtttatatctctttgctttttaagtttgatcagatcagagaagatatgcttgatttttttttatggtactaaaaagaataaattttttagtacaagcttatgccatagggggagatatttctcacacacacaattatgatcatatatgagaacaagttaatacccttttcaactgagctaagcccgttggtttgtttagatttatttcttgattatggttttcatttttaactcttaccaaaccaaaatactggatttttagaagggaataaaaaaccaaactcatcgacaaagtcaacatgaatttcagataaattgtacaaagccaggtgcactatgattttttttccccatctggttcaatatttgcagtaatatgttataagtgctcatttggggctaactgttttcacctcattaccacttaccatcctaagtcttagccctgcttcgattgaagatgtcttatgggtttgtagggcaagtttattttgaagtttcttttttgtgtctttgggccatttcatttttgtgtttatttaaattgaaagaagttagtgactttcaccgaaatttcacatttaaaaaaatatgtttttatagaaaataatagatggtttatttgaattaacgtcagcattgttgcgttgcatgtaggtccaaataaaactattttaatccattttatataaaagtaataataactaacttgcccccatttctctatcgaaaatacagaaagcttcaattacaagatcccaacacagtgctattgaaaacatacgggagaaaatgtattccacgttctctaaactaaaatagcatgacctgcacaaggcatatacaacttgaaacttctccaccaatatatcttgatgcgcagttgcatttatggtagtttaattatccttcaccattttaataaatttgcttatcactttgtattttctctaaatttcaggcttccctaatttctatggttaagaaatggtgaaagatgaaagtttactctccttatagctaatcttaatttctatggttaagaagtgaagaaagatgaagatttactctccttatagctaaataaaattaatgtattgtaagatattagtgattatatgtcttaactcgatacaaaattaaaaaatattgaagggagagtaactttgtgttgtatctaaacctatctattacatgcagcctaacatatttctctttgagtcaagtattatatttagaactttgcatcaatgcagcctaacatattacttgattatttttatataaaaaaatttgaaaggtaagttttgaaggtaattatccctcatttgttgtgccactaactcaagtcttatgaagtttagattttctcacaccaactagtgttgagattttctatatttatacacagtatgaatcagttacagaaattcaaaagagctgatatcaaggaggcgttacacttgctatgatcaaggaaagattacaaaataattacaagattcttaggaaggagagactacagctggtgctaatatcatgggtgatctgcagctgttacagctgcatttgtaacggttgaaggtgcgtgatctgcagctgttacagctgcatttgtaacggttgaaggtgcaactaatacgccccctcaagttgagcgttcgtgaactctcaacttgtctcggatgaaggtgaagcgaggggaactcaatggtttggtcatgatatcagcaagttgatccttggtggagataaattgaacactaagctttcctgcagaaacttgatcgcgaacaaaatgataatcgatctctacatgctttgtacgagcatgaaaaattggattagcagaaaggtaggtagctcctaagttgtcacaccataaaacaggtgaagtagatgtggaaaatccaagttcctggagtaaggagcgaagccaagtaatttcagtggaagtattggcaatggcacgatattccgcctcagtggaggaccgggcaacagtatgttgctttttggctgaccacgagaccaagttacagccaagataaatagcaaacccaccagtggagcggcgatcatctggacatccagcccaatccgcatcacaaaaagcttgaatggcatgagaagaagatttgctgagaaaaaggccatgagagatagttttcttgagataccgaagaattctcttgacgctagcccaatgttcactggtgggattgtgcataaattgacaaactttgttaacagagaaggacagttccgggcgagttaaggtgacatattgaagagcaccaacgatgcttctgtattgttgtggattagataatggtaaggagttgggttactccccccgtagccaattggttttggggtggaacctcaacttccttcatggtatcagagcgggttgcccacgtgtgaaagcccaacggccacacgtgctccacgtcacccaaaatgtgttgtccacgtgttaggcttgaaaattcgccacacgtgcgggggcgtgtgagaatgtaaaggtaaagagtcccacattggaaagttgagaaacctagcaagggcttataaggagttgggttactccccccatagccaattagttttggggtggaacctcaacttccttcaagtctatatcattgtatatcagtgcgtaggaagtataaaacacgtgttgcaccactttcccactacttcgattactttctccaattaaagagtttcacttgagcgatctctcaaatagatatctttatagactctattatgcattgtattttaataatctaaaccatttagtttttagatattctctcaaagatcatcgctagaaaaaatcacttgaatccgaaaccatttaaccactcaattaaatggttgttattatagtcttttcttgaagcaccgtatccgtctattttgttggtcccaattagatgttttaatgaaattcaatttgcatgattttttgcaagtatgatttatgaataaagatttgaaaaataaacggtttgaatagttgaattttttgtttttttggggtaagggatgtatcaatagaatatgattgattttataggacacaattagatgttgaaacggtttccaatttttctaattttttgtaaggatgatctataagtgtagacttaaaatatatatagtttggatcattgaatatatatatattttttttgtagtttgccttaaaaggtgtctctcattatatatatcggattcaagtattttttgtaggggtgatcacggttcagattggatcgggttcacctcaaaacaacggccaaattaattacaaaataactgtttggttcggttccattttgacaaaagtcatgaagaaaactgaaccaaactaaaccaatttaagacagtttggttcgacaacctataattacggtttggtttggccagattagtgccccctagttcttagtaaagatgatatttgaatgaatatctcaaaaatagataatttgaattagtgaaatacaatgcggagttgaccctataagagtgtctcaaataagcttattgtgcaaaagccattgtaaacatgaaggaaagcagggaggggtagtagaaaatatatggcatgtggcaagataatagcatttggatgtctgcatatgagattttttttaaaaaaattattattattattattttttgccatcaactacagtaaacctccaaaagtatgagagtcattactattattggcaaaaatcatgaagaaaatcgaaccaaactaaaccaatttaagacagtttggtttggcaggattaatgcccacctctagttcttggtaaagatgatatttgaatgaatatctcaaaaatagatgatttgaattagtgaaatataatgcggagtggatcctataatagtgtctcaaataagcttattgtgcaaaaancattgtaaacatgaaggaaagcagggaggggtaatagaaaatattggcatgtggcccgataatagcgtttggatgtctgcatatgagatttttttttaattattattattatttttgccatcaactacagtaaacctccaaaagtatgagagtcattacatactcatacccaacctttaaagactcgtttgaacggtactccaacttatagaaaccttacgattgagaccctctgttacccagtatgttagtagatcagttaaatattgacgtggcaaatttaaatcctttttttatttttaattttatactccacataggaaaaaaaacaattaaactttttgtattacccttttttagcagaaaattgtgtattattgtataaacaatttaaaaagacatttttttgaccaaaaaaaagaaagagatcttttatttaaaaaaaaactcaccggctctgtttgctccaccaaccccttatctctatttgtacaatagcaaccatggaagctttagcggctctcttacgcatcagcctcgccgtcatgagccgaaacctcgccttcaacaccaccgagaaaggcttccccatctatgaaaacttgaatcaaattcagaaaaaaaatgggcaacgatatgagcgacaaattaatatcgtcaataaaggaggcattcacgctctcgtcatcgacaacgacggtaagattgttccttcggaattagggaacctaatgcgattgcttggagtcaactcgacccaggcccaacacaaatccatcgctgccgaggagaagctcactgcccccttcaacttccctcacttcctcgaccttatgggaaagcacatgaagcccacgcccttcgatcatcagctctgcaacaccttcaaagtcctcgacaaggactccaccggcttcgtctttgtctcggagcttcgacacatactcactagcatcaacaagaaattagagccatccgagttcgcaagtggatctgggaggtcaatgtcgggtccagtgccaagtgagaagctgaacggtgtgttttcactggttaaggcccagtttgggattgctatcacttttttaaaaagttacttatgttgtgctttgaaaataattagttgtacagtaaagcaactccatatttggtaaacaatatttttaaagtgttgttaacacaaaaagcagtgtcaaaactgtttagtaaattttaatataaaattattgtaactgtgaataatgacgaaaatagacgtgatattgaaagtattatgtactaatcatgtggtagtagtagtggtgatgaagtgaaggtggtgattgtggtgccagtggcaacgtggtggtgttggttgtggaggtggtggtggatgtggaggttttggaaaaggtggtggttgtggtggtggtgatagtggtggcggtggtggtggtggaggtggtagaggaggtgatggtggaggatgaggaggaggaggatgtggtggtggtggtggtggtggtggtggtagtggaggcgatggtggtggtggttgtggaggtattgaatatggtggtgcaaatggtggagttcgatttggtagtagaggtggtgtcatttttaaacatgaatgatggtgttttgggaattaaaaaaattcattaaatgttcatctttgcttcttttgaaagcagctttgaaaagcaacacaaagcctgcttttaaaagctgctgtcaaaagaccattgcttttaaaataattggaatattttatttttaccaaacaccttaaactgcttaactttacagtgaagcaggtttttagccaaaaaaaaaaaagcaatcccaaatggggcctaaggggtttgtggaagtaatttgggcctaatgtatgtttttcttatcttttgatatggaaattttagggtttgtttgttttggattttgtttgtggtgaattgaattgtctctagtacatactagtaaatttcaaggtgttcgtgatgtaagtgttcatggtttcatggaatgtaatatggatttgttggattattttgtgggggttttgtgctgaatctttgttttctgttgtgaattgctcttgatttttggattgcatttgcatgtcaagcaagaagcaattgagacaacccttcatcattgctaccttagccacctggcaccctattttactcccaaaaggctacacttctctcgttcctcttggacgactttttcctgtaagcagccgtacacccgattgtccaaattatcaactcctgctacctttgctagtgaatcccctgcagtttcagctcttcttttatccttttgccaaattgttctttgatgcactctcattccattagttttaaccacatgcgagtaagccacctgacctataaagaaagggccttaaaacagcggtaaatgatctaaataggaatcagttatcttttaaaaagagaaatgctagcaacattctctcttttttgcttgacatatgtcattctccttacacttaaaacaatgtcattaatatattatacaagctaacttttactttccaagtttaccctcattaaatgtattcaatttatccaaaaaaatatcacaactttcttaccatctttttttttttttaaacgtcaattatttttttaaagaaaatgtctgtttttttcaacaaaaaaaaaagaaggaaatttcctcttttttttttttcaaacacaaaggatttattattatttttataataaaaaaaagggacatttcctcttaaaagcatatattttcttttaaaaaaaaattgacgttagaaaaaaaattaataagatggtaagaaagttgtgaaaatttttggataaattgaatacatatattaagagtaaacttggaaagcaaaagttagattgtataatatattaatgacattgttttaagtgtaaggagaatgacacatgtcaaagcaaaaaagagagaaagtccaaaagagaaggttagagagaaggttgttagcattcctcttgcaaatttatgacggccaaaacatcagttttgatcttttgtggtttatcacttttgccgctaaggtccttatggttttaatttgatcacttttgcttcgatggttttcaattccaagcaattggaggacatttcctaattcctgtcaaattcttctaactgccattacttattgaggggcatttcagtccataaattattaaaaacttcattgcttttcaaaagtttaaaataatttttttttagagataaaccataccattacttatgatatagataaacccataccatttaatttttataaacaaacaaaaaaataaacgaaaaaatgacaactggccctattgaatttaattttaattattaaattactttgataccataatgagtattttgggttttttttttcatgaggttttggggttgggtttgttttaagaaatcgatagaagttttgtaatttgtatgaagttcaaagcctctttattatgttaaagaccccggtttcgaccacaatcttcgaagaattgatttccaaaaagacccagaacccgaatcagccttagattttgacccagataccgtacccgactcagactcagaccccgactctggctcagattctgatccatcttcctacttaacctatatcttatgcatcttccactgcacatattacaacttcctaaccaacgacccagacacatggatgagattgcatatactgtggcaatccaagacacactcgcgagacttgtttcaaattaaatggtgaccccaaatggtgggctcttctcaagggatatgacacaacgtggatactgccagtaatggtactggttatggattccaaatttcggataaaagtgatttcacaggctagataattgattctagtgcaactgatcatataacgtttgatcctgatgattttctgaatactacctaacccgaacgagcttatatacctaatgccaatgggagttacttatcatgtgacagaggctgggcactgttgcaatctcatcatctctctcattgtctaatactttacttgtcccatctttatctgtgataccttgaaaatttccagaatatttttatgcgtacaataaaaattgcacggtccattttgaggtaagtggcatgttttcgaagtaaatattgtcattatgtttgagaataacttacttctgaaaatataaaattagaattttagtgttttaacttcataggaaaattgtagacgtcaatatgagcttgtggtattttacagataatttttcggaccttggacgtatttttagtgaacttttgaagttgaaaaataggaaaagaaaataagtttgacacatggcgttttctgattggttggaacacatcaccaagccacatgtcaacacctgattagttggaagacttggaatacgttgatccataggttgactacggtgtagagtactattctgtgagctagtctcatctaagccattgatctgattctcgatcagatatggactgtagatcaaaatcaaagacttttgacttgatctaggcgtttggtttggttgttgggaatgcatctggaacatccatcttaaagctatcatgtacctccctacacctgatctaaaactcatcttttaatctcggccttcgaattaaatattcgaatcgatccgaaccgtcagggatttctataaatatccccaatctctcattttcttcacaccaaaattcaaattttctcttcctcccactcttcctctacttctcctccattagagttactattccgacatccttcagagttaccattcatcaagcactattcatcccaaacactatttacccaaaatttactgttcacttccgtacgtagtataagccgtttcttattcattccaagaagtcaatattgacgtcagatttcactattcattgttacactgaccgaggtaatctctactctctaccttgtattctattcgttttaattccatatttctatttcctaagtgttactttgcccaatttaaaattttcaagtatgcaattcgtattattttctaattgggtaattttcaagttattcctaaaatatactccatacgccgttacttcgtccatttgaaatctaaacaaactagataatcttataatcattttccttctataattcctgaaccaaattatgcatattccttttgtccatatgtatgatcaacaaagtactatttagactttaactttatcgtaccgtgaattttaccttttttggaccagagatggaactacaagactattgggacatttttggacttttcaaaaagtacaaggactattttacaattgcttagaagttttaaggacttataaggacaaatttgtaatttggaaagtgcaattgaagacaagaaaaaggactaaagtgcaattaagaatttttcgtattatttctgaatgttggattgtgattcaattgctcaagagtgaccagtatgagttcaaaggtgcagatggcaagtcaggcttgagctttggattccaagtagggggactcactctcgaaattttatttttgtgtgtatatatgacatagtaaatattgttgtgtgcgaaagatgcactgtttttggataccatattgtttggttttatggatgcttggctttgaatttccaaagttttggaggatatatgcatgtgggcattatcctcgacgatttgatagtaaagtgggtataacgacttccttgggtttcgattccctaaacctccggaggggctgcgcagactaacttttccaatattgtacgtgtgaggtttagttgaagtagtatagccgataaatggacactcttgctattgtaccaggtaagttggatatatttttatttctggtagggttagttggcctttaagtatcggttgtcagacaagcatattatgggtacctgaaaaatgtttgcgaaagttgtgtgagcgtgccaactgtttttggatcctatatttatatggaaaatgatttgtgttgtgaatgttttttttcattgtttctcttgaatagtactatgttgtttcgctatctttattaaacctggtagnagtccccttgctctgtgatgagagtctcaaattacatattttaaatataattttggagactttatgatataaatttggcttaataaagaaaatttcacttttgtaaattaaaaaaaaaaagaaaaagataatttcacttgtcgttacatttcattcttaccaattggatttatgaattttttgtattaaatttagtgaacttattaggtgaatttatcttttgtaggaagttggaaagccaaaagatgaagatatatatttgtccaagtttaaacaatgtgtgaagagaatttttggtggattttattgaagaattgattgtgggattattgtgattataaagaaaagtaaagagggagaagagaagacaaatccattaaagctaaaaagcctatttcatgtcattcatagcctttaacgcccactacctcattattatttcttttccactaactctccaccatcaacacatgtttaatccaattaatccctcacttaaaaccattcattacccactatgaatgtattaactttccactcacgactactataaatcacccatcaacttatttgccaaggacacaactacgacatattcctttgtaatgcgttgctagaactgtagctaaggctaaagggaatgtcctttgaataagtttgtgaattatttttttaatccaatatacggtatgaattgcttaattttttaatttttggttgttcaatattttcatatctttttattttgattgatgataagtgctactatcttttcatgagttcaattatatttttctcattgttaagaactaactttgcatacttttaaattaaatgattaaatattaagcaattccattttatgaacaatcattaataaaagataacattgaaaaattaaattggtttaaatttatttagctcccagttaaagtttctcaatgataattttaatcgaattataatttggattaaagagtatgaaatatattttgcttaaaaaattgaaccttgatgccttagcatttccattattgatatttcatttttagactcaatatctacgacactcaaatcaaattaattttctatttggcttgtcacatttattttggtaagtttaattatttgtttgtgtgcagacatttgcctacctgctttttgaggatacgatacttggacttcttaaaatatatgagtgttatatcatagtttttattaaaataataaaatatggagggccctcaaatataggtatgcattgctagagattgaaaaatttagctttacactattctttaaaaaattaatattttgaagggctaaattataaatgtggactacatttagccctataatcggagatggcctaagagcattttcacctatttgccatgacaaaggggagacaaaggcttgttactattcacatgagtaatgcatgtcctagcaattttttttattgggttttgtaagatctcacatcaaaccaacaaagagggggtgatgtgccttatatatgcacatctgcatccatctagcacgaggccttttgggagctcattggcttcggagtcgtaggaactccgaagttaagcgagttgggggctagagcaatcccaggatgggtgactcactgggaagttgctcgtgagctcccagaaacaaaaccgtgagggcagagagaggggcccaaagccgacaatatcgtgttacggtggagccgatcccgggatgtgacaatttggtatcagagccactctgccgtgtagtgcgagtgtgccgacgaggacgtcgggcccttaaggagggtgaattgtaagatcccatatcaaaccaacggagagggggtgatgtgccttatatgtgcacacccgcatccatcttgcacgaggccttttgggagctcactggcttcggagtcgtaggaactccgaagttaagtgaattgggagctaaagcaatcccatgatgggtgacccactgggaagttgctcgtgagctcccagaaacaaaatcgtgagggcaaagaggggggcccaaagcggataatatcgtgctacggcggagccgatcccgagatgtgacagttttctacctattgtcatgacaacccaagagtaacgctactattcacatgagatataaagagaggaattgtatagaattttggtatgggaagtgaagaatatagctaggtatttattaaaaaataaattctgaaatttttggtatttttttttccaattgcttttttttatttaaattaactgctgacgtcacaatgacattaatattagattagcttgcccagataatagtctagatagtcttgcctttggacttacccaaatttatagagcccatctcttaccctaacaagaatggggtgttagaaacacaaatagggccggagacctctttggctgggctggggcctcacgctgaaagtgctctaacctgagcagagtaaagataaagctaagtgtcgcttggtgggttaaatcaaatgacaagacaggcaggacctgtatcataggccatcctcctccacataaaaataaaataaaactcccttcttgttttttcgtttgtagcctgacacggaataaaagcttttgtatttccataaaaaggacaaattcaattttaatcctttactttcttttacaaaagccagccagcggcagcagccatttctctctctcattctcttagtttttctggacacaccaaaacttcagacctgaaacctcccaacgaaggtaaacattaaacaaagggaggaaaacccagaatttaattaattaaataattgattaattgatggttttccctctttctcctcgtcatcttcttcatcttcttcttcctcatcttcttcttctttgtgctttggtactctaggcatgttgttgtgaactcgtgacctattttggttggtgggtttttctgggttttgttcactcagatctgggggccattgaatgtttaaaattgagaagaaactcagaaaatattgtgtagttttttaatgtatggaacataataatatatatgaaaagtggttggtttgaattttttttgaaggggggatggtgaagatgatgaggaagaagatcaagatcaagaagattgattacttgcctgcaaggcaggtgaccttctcaaagaggagaagagggatcttcaagaaagctgcagagctatctgttctgtgtgaatctaaggtggcagttgtcatattttctgctactggcaaactttttgattattccagctcaaggtaccaacaacataccttataattcttttgacttttccttccttgaaaaataaccgatatgatgtctctgaagtctgacttgtcctctctctgtgggtgagagagggagagaaagatcttctaagattcagccatttgggtcaaaatatatttggaggagaagggtacctttatggaattttttatttttattttttcctgaaattccatggtttgatttgaactgtggtctagaatgcaaataaataaaaaaaggagaattaaaaagaagaagaagagagagagagagagactgtatttcaaagggactatttcgaggattactaccttatacctcaggaaagtaaagtttactattgattaagacaactgctagctaggtgtccccagtttgagaaacattattttattaggaaatgattgaaaccttaaatttaattgttcccctccagagacacgggatgttagaccatcaatcaaatcatatacttaaacaaaaatacatatcaccaattctagattcacagtctgaccacataaaatgataaatagtctatctagtaagagagtattttacactttaccccatatatatatatatatatatatatacatatggtaaagatagtggtgggttgtgtccgaaaaggtgtccttcattctccccacctaaactaaaaaatgccaataaattaatgcggttacaatttgctaggttacaaaaattgaattatatgatttgtagttttcttactaggaataaagtgatatgatgataactagggaattcatcattgaagcattgaagactttcagcaggaatatgcatccttgctctcaagtatgagaaagaatttcatgaaatggggataacattattttgttatccccggccaataaagttatgacactcgtgataacacttctacgtggcatactattattggttagggatagcaaaacaatgctatccttgttgcaactaagtttttctccctcgagtaggacaatgggtggtggagctatgacccataacatgtttttgccatgaatacatgcaagatcgtgttaaaacctactttcataatcagatgtgcatgcgtgagaggaaggaaagggagagagggtgagaatgaaatgttaactatggaataagagtttaaataaggtcatgcatcctttcagttaacaaaattaattgcacatagtaagcgctgataatttatattaaatatttttaaaaaaaaactagaaataccacctctaaatgtctattgtgtggaattaacgattttttttaatatataaaaagttattataagtggcctttatatatataaaaaaagccttgagacaaatggaggtctcagttaaatactaatggcaatttgtaattaatttcatttaaaagttatttttctaggtttccggggcctaactgacatgttatatgcaaagctactactagttcgtctactttttattttttattttaaaagtttctctaaattgcagtccgtgtttccgtttgagcatatgtacaagttaccctgtttccatacagagtacttgcaaccaaaacaaaaaccataaaacgactttactgtaatcaatatacattgaaactaaacaagaataaaagtgtagttctttagccttagattttactttacattatttttctaactgtaatttagaagcttagaaaagtattcctctacttacgttatactaaattttcgaaaaatttattagtaatgtgctgctgaaagtcatcgaaaaaactatgatgctaaagcaaatgattcattactagtaaagtgtcatataggtattctcgaaactccaaagtcataagtgaagtttaatcaataatacaaaattgtttcagttttcatttctgtgttcacgaaactcatccattgttatagttttgttttaagatgttttgttcatatttttattttttgaaactcttaagggcttggagaggccatgttaacacaggatatagcatatgttgtctattcttagtcatagcaaattgtatatactgacatgcttaagaaccttagccactttcactaagttttcctttataggagatcattcctcttccccatttagtaggttcaccacttcatagtagatcatcttcttcccattttagtaggttcaccatttcaattgcatctgtctaactagttcgggatgtaaactaaagttgtagggttttccaagggcatgtttctctggaaccactagagagcgcccaacgtttgctcttgtcaaggtttggttgcaaagctgcatcgtagctttgccaaatttagaatccacattgtataacgagatattatatttatctcgttattctttctcctagtaataaggtaaactccttggtcatcttcagtttaaacaagcgcttcatcaaatactattgttgcatcacacatggctttctcatacatctcggatagagtcatcatcaatgcataagtagtctcgacacctcacttcattcgtaagcgtcaattggatagctgcaaaggccataccatcaagctcatcccattcctcttgttccctgtccttaggtttttatttttttattttttattttaagataaaggaacatagagtttcttgagattgaggtaattctcgatgtgagaactccgaagcaagaaatctctaccatcaaacttgtctactttcttctcaacgttgtcgacgctcattgttcgcacataaaatgccaagttctgataccagttaggaaatctatctcttgattccataaaagaacaagtgtgaaacaatataaacgcgatcgagaaagacgaaagtaagcaaacacacaagacaagaatttgttgacgagattcactctatatgagctaagctcccaagttgctaaagattcaccatataagtggaagggaaaaacaaaattaaagtaaatggacgggttgagaatcttgtgtagccaagttaggcacgatacaaccagaaggtatttggatgggcaagcaccaattgtggtggacaagaaaacgagttaccggcctagctcatttcgttatacctaagtgagagcatgatgcgctttgcaagttatggacacaattttttagggagagctaagcatttgttttaatattgggaaaattcccattcttaataatcgttcaaattaagttgtggtagaatattagacagctttaaaccatttagaggcgtttttaaatgatttcttcgccttattttgaggttaaacaccgtggagaactttgtttttggataaagtaatacacggaaaatggtataaattatgaaataatataattgtaaaaatcccttaagagtaaggatccctgatttcacaagggtgcaaaatgcatttgaccctgaattttttcccaatacgtttggcatatatggccctacatagattccaacccatcccactgactaaaaaggggacaactaaagataacatgattccaaagcatattgcaatgaggtccccagagttgatgtgagaggaactgtgactaagatttttgaggcccactccttattagttgacttgatatggtctcacgtcggtttgccttgtgttctaaaatatctcagctacctgacgtcttgatttgcacaaccaaaccaagcaataaagccagccatgcggcagccagcaaatagcttaaagttctcattgtgtatgtgattttgcgtgtttgaaacaagtttttgggccttttggctgactttgcttgcaacaagaccacatctcaaataaaatgttaacttgatttcaaaatctagaaaatcttacttgtgatgatggtatgaaatcttggttgatggggtcagattattttttccttgggaagggtggggtaaattagttcctctgtttttcagtgttgatgttatttgttgtaataaattctaccagttagcttatttcatgcatgtcaaaactggtttggttattgttgatgtaattttttgtaataaattttaatgaaataggcaatacatacattacacttgctttcttttgagatgattggttttctatggtgtggccctttttcggctctgctgtttgcactttgcattataattgaaaatgaaattatgggtttgcattgtaattggaaaccaaattccaaaacaacaagttatgaattgttttgtagttggcctaattaatggtggtgggtcgttgggggttaagttgtgcaagaatgctaattctggttgctgattatgcttgttaaagaagctcaagtatgaaaagcaagatgccgtgccatcatgctttatggcttagcattgaagttatgtcagtgagcgaagttagagaactttagtatgaaaaaattacagattattagaatcctgaatattccttaaaattggtctcttcttgaaatagtgtgtttttttaatacaagcgatagtctaaattaatctaatctatgggtaaagggattcaaacttgggtgcaagggaggtgggctcactgccctggccaactagcctaacccatgtatgttcaaatagggcttcttaaagggaagcataccatataatagtatatacaaaagaaaataaacagaagtgtgtctttgtgtttgcagaccagaatttttcataaggggttaattattttccttttcttttctcagcatgctttgtcacatgtattaatttgatcttctatgtctactaacttacactagtgaacatgccaattgattgttttttccttttgcgttgtgaagtatcaaggatgttattgaaaggtacaaagcacacacaaatggtgtcgaaaaatcggacaaaccgtctgttgagctacaggtaaatgagttcaatgttttaccttttccatttccccctttaattgttctgcaattaattgtaacatattctgtgatctgtactttggagcatcaaaatgtatgtctatagcatctgaaggttctgattgtttgaacataaatacaaaagaccagtattattttctgttacatgttagacttatgcatatgcattcagggccttcttggaataaatatatgtaggtggcgttggtaagtccatttctagtagggccaagtcatagtctggtttgagaatacaaatggagcctatccactgtcaatcaatcagatttttattgtttacttctattactgttaattgtttttcaagtcttttttactcaagtgataggcaagatgctcttgaccacttgatttataaactcattgtttttcaagctggatttgtaggggaatgcgcagccaatttaaatttcttatgatttcttcaaaactccagatagttaagttggagactgctattgaatcgatattacagtctcctagagtattacagtctgtttttggaatctaaaaaccttttggtttgaaaaaataaaaaaggaaaagaacagtcatatacaagtctccaagatcataataaaaagacctgaatataggtaggttccccaataaatctgtaattccatagaaatccaatgatattttggttgccttcgaaattttgatggagttccctttggaaatctagcttattatgtccaaatccaactatagagatatgattacaactccttaggttttaaacttaactcaatgcaacaaaagttcttttccccactttcttgatcttatctcaaattcttgcaagttgtattggctggtataagatctgagggagtttgagaacgatgtggaatatgtttcctgctcctggtaaactatttttctagagtactgaattgacatgagtgatttcctttcataaatgtagtacacaattttactaccatatttggaatgcaattttgacaagtatagattagtggggggagcccatagacttaagtacaaacagtttttctttatcatagctctgcccttgcctagaacacagtttggaatgcaccagagatgaggaaactctcaaattttattgctaagatatgataagataatgaatacgatttccataaggtggtttactgacaagaaaagaatattagaatcataaacttaaatcttaccaagaaaaaaaatatcctactgattccaaatctagaaacaaacaatatattcataaaatcttgttgaaaaccatttttcgtaaaagttgctcaagcaacctgaaaattccaacttatacgccttttctatttcatctagcaccataattgatgctgaagcacaatttttccttcttaagcaatttcttgctaactttgagcggctgtctcccctttaatttttcccacttcaactgacccctatcttgtcctacatcaaatgttgtgtgtctctctcagtgtttctgagatccaacaccttgaaacttgtttaatctctccacgaagggtgcctcaaacctgaaactctcactttgttggttgcatttgaggcttaggctcatatagaaccctaatttcgttagctacctaaatcctcacttttttggttgcttcagcaccaaaaattctaaacacgtgtagagaaaccccaaatttggagccacttagctcaagactaagttgaatacttaggtcaaaatttcattaaactccatatctctatagaaagaagttgagtttgatggcttttttaactaatagatagacgggagtggataagaaataagtttcttaccatctggagattctgttttggtgctcaatttaatcagcacaagttcttcttccatttgccttaatctaattttgaattgctctgcaagttgggtttgtcgctattaattaaatctccaagtgtttctaaggtattaatgactaggacaaattgtaaatccatgttcttgtatgttaaagtaattcatttgattccattttcaaaataaataaataaaatgtaatccttttattttcccatcatgttgtctcattgtttctttttggggcttatgtttataagcttttcatgtagcagagacagattagattaccttaatgatggtttcatttctcattttagctagagaatgaaaaccaaatcggattgagcaaggaactcaaggagaagagccaccagctgaggcaataatttgttaattcatgaacttgtaagatatatgtgggtttttagctggtattatatcgtttgatgtatctgctgaaattggagtggcttgcaacaggcagatgaaagcagaggatcttgaagagctgaattttgatgagttgcagaagttagaacaactggtggacgcaagccttggccgtgtgattgaaactaaggtctgccgtcgctaatttcttttctagtaagcatggtgcgaaggcattactctgtattgtcctttgtgcaaagaggaaatataattgttcttgttattgaaactgggttgtcccttaggttttgagttgctattatagctggttacatatatgcaaataaactatacgatgtaaatgtaaatattccattgcttttcaggaagaactgagaatgagtgagattatggcacttgaaagaaaggtgagacgattgatatacgtactctgtaatcgaatacaaatttcaactatttacaataatcaggactttgaaaaatacatatataaattaacatcaatgatcataaccgagtttggctttggttctccctttcatgcaccgactataatttcagggagctgagctggtagaagccaacaaccagctaaggcagacggtaagtaactaccacaatcatgtacatatttcttctatttgtttgttttttttatttgtttttcaagaggaagagttctcttaataaaagatgggttgatcttttttttcttttgtaattttttgcatagcagatgatgttatccggaggaaatactggacctacgcttatggaaccggagaggttgagtaataatattggaggtggaggagaagaagaaggcatgtcatctgaatctgctatctccaccacctgcaatagtgctctcagtctctctccctctcttggagatgactccgacgacgtcactttatctctcaaactggggtgagctagcctacttttgctttgctatatatttcctatttatatacatatctatatatatgtatattatttatgttgcgtatgtctctttgctttttaagttttgataagatgagaattcagagaagatatgcttgctttatttagaaaaatgaataccacagggtgtcgtgtaacaaagatattttcccttcctggtgcggagagaagatatgtttgcttttagatttatttctctacttgtcaacttgattctgcgtttcatttttaactctagcaaaccaaagcgttggattttttgtcgacaatatttttttatgatagaaatgtcaagtcatcatgaattctagatacttgtacaaagccaggtgcacgatgagagtttttttcccatctgcttcaatgtggtgtaagtgctcgtttgggcccaactttttcacctcattggggcccaaagttttgggcttgcttcgattgaagatgttttagggtttgtagagcaagtttcttttgaggtctgtctttggggccatttcattttttgtgcttatttaaattgaaagaagttagtgacttccaccgaaatttctctccctcgactaaaatacgagagaaaatgtattccactctccctcgactaaaatagcataacctgcacgaggcatgtacaacttaaaagttaaaacatctccaccaacgtatcttgatgcgcagttgcattcatggtattttaattatccttcaccattttaataaatttgcttatcgttttactatgaatttcaggctttcctaatttctctggttaagaagtggagaacgataagagtttactctccttgtagctaaataaagaaagtcgaggtattataagatatcagtgattgtgtgtgttaactcgactttgttttgtatctaaacctatgtattacgtccagcctgacaaattgcgctttgacttgtattgtatttagaactttgtatcaaaatagtctgcatggagactcttgtggattttgtattgcttcaatgtcacagcacaggataacgttacgaaaggattatgaggtcttgtttggttggtgggtcagtgtcatagacattatttatttagactattacaactcatgtcacgaaacttaaactagacaaaaagaaggtaaccaaatcaattttgaatgattacctgcatatttcctaaagattacataatagtatattcgcgagattcgtatttttttcgtggattaaatgcgtagaagtggacagggggaagataaagacaaagccaagtgtcgcatggtgacatgacagcgcatgacctgtgtcaaaggagttaaaagcccctgcataaatatctttttttgtgttctttttcttttttggtagcctgacacgtaataagaattaaaccgacaactttttaaaacaaactaaacttagatgtaagacgccacattttctattcccacgttacaaaatcaacggtggagattgcattcatcaacgtcgagagcatctgtgcccgtcgattcgctcaggtttgttaacttgtttgcccagaggatacccaaagtaaagcacaggataatagaaaaatatatttttttccataaaagggaaaattcaatttaaatcctttcctttcttgaacagccagccatttctctctctcgttctcctagtttttctgtacgcacaacttcagaccctgaaggccccaaaagaaggtaaacgttaaacgaagaggaaaacccataaattaattaattagggttcttctcctcctcttcttcttcttctttgtgctctggtgctctggtgctctggtgctctggtgctctagtgctctaggcatgttgttgtgaactattgacctatttcggttggtgggtttttccgggttttgttcacatagatctgggggccattaaatgtttaaaatttatagaagaaacccagaaaatattttgtagtttttgagtgtatgaacataataatatatgtgaaaagtggttggtttgaatttttttgaagggagatggtgaaaatgatgagggagaagatcaagatcaagaagattgactacctgccagcaaggcaggtgaccttctcaaagaggagaagagggatcttcaagaaagctgcagagctatctgttctgtgtgaatctgaggtggcagttgtcatcttttctgctactggcaagctttttgattattcaagctcaaggtaccagcaacacccacccctataccttataattcttttgacttttccttccttgaaaaaataaccaatatgtatgatcatggtgtctctgaagtctgatgacttgacttctctctatgtgggagagggagagggagagattctgagattcagtcatttgggtagaaatatatttagaggagagggagggtgcctttctggaaattccctgtccttcctgaaattccgtggtttgaactgagctcacagacattatttattgtgtctaaacttccgaaagaaaaaaaggagaatccagaagcttagagagagagagagagagagtcagtcttgtagatgatacttttagatattacttttttggggtcgatttttacttttagatatactctgtctcaaaggcaactagttcgagggttactaccttgtatatataccactccaagaaaatagtctatttttgataaagaaaacttctagttaggtgtcccagtttgagaaacattatttttttagttttccatgtttttcatgcaagatgtggtgtatatatggtgatgttcttttttacgtgcaatcaattttgtttaccatatgctaaactctgtatatcttgttcggtaactggttttcatatatactcgcatagtttagtatgttgctgcttcaatatcatgtcccacctaacacatattggtaatagataatatatgaaatcttaggtcgcgcttatacagtgaagaatatccctgtccaaggagctaaaattaatggttcccttatgagagatacggtatgtttgatcatcagttgaatcatacactcaaacaacaatacagatcaccaattttagattcatagtctgaccacataaaatgataaatttgctaataaaatagcattttactctttaccatatatatacatctatttgtatatatattatcctttaaacttaagatagtggcgggttgtgtttgaaaaggtgtcaattcattttccccacctaaactacaaaatgccagtttggccaaggggaaagtagcacccaactccttataagccttcgttaggtttctcaacttccaaaaattattgtgggacatttcttcacattctcacacgccccctcacgtgtggcgacatttcaagcctaacacgtggacatcacattaggtgacgtagagcaagtgtggacattgggcttcacacgtgaacaaacccgctatgataccataaaggaagttgaggtttcaccccaaaatcaattggctaataggaggagtaacccaactccttataaggccgtgctaagtttctctaactttccaatgtgggacatttcttcacattctcacagttacattgaattatgtgatttgtagttttcttactaggaataaagtgatatgatgataacttgggaattcatcatagaagccttgaagactttcagcaggaatatgcatgcatgctttccaataaggcaatgggtggtggagccatgaccattacatgtttttgccatgattgcatgaaagattttgttaaaacctagtttcatacttagatgtgcatgcatgagagggagggtgagaatgaaatgttaactatggaatacgaatttaaataaggtcatgtgcttcctttcagataacaaaaataattgcacattgtaagcgctggtcatttatatttaattaaaaaaaacctagaaatactacctctaaggtggtcttctttaacccaaaatacactccatatataaaaagaatactcttatatggcgatggatttttttataaaaatattctgtattgtgtgcacgaattaacaattttttttttaatttaaaaaaagttatcatctaaaagctacatctatttgcgttggttattatcgaacaacattaaaagacagagaaatcaaaactgtaagtaacaaacacgtactaatggaggtctttaattctttatgtcagagtgcaatttctaatcaatttccgttgtaaatttaatttaaatgttaattttctaggtttccgggcctaatttgtctactttttttattttttctattttttaaagtttttctaattgtatttctattaaagctctcgatattgcattccatgtttccatttgtgcatgtgtacttgcaaccaatgcataagccataaaacgatcttgcattgaaactaaagaagattttgctttgcattatttttcaaacttttccaagtttccaagtagagcaatgtgtcaaacgtttccaagtaaaggttccatgagactcatccattcttaatgcacggggcagaggttgaagtctaaaacctatacatgacctagcccgaagcggatagcttttcttaaggtttttaagggtttagtaatgattcatctttgcatggtagcctaccaaacctaacaatatattcaaagcatattagtcgacttgatggtctcagagttgacgtcagaggaactgtgactaagatttttgaggcccacttcttatcagttgacttgatacgtgggtttgctttgctttgtgttcgaaaatatctcagctgctattgaaggaatttgacttgacgttttcatttgcgatatgtgatttgtgtgttttaaagccaagctgatattttttttattttttatttttttggtttggggggagggggttcttagcttattgcatgcatgtgtagtttggttactgttgatattactttttgtaataaattctaatgaaataggtaatatattctttgtacttggtttctcttgagatgattagtccctttcttgggtctgcaatatataattggaggaaatcaaattttgggtctgcagaaaattggaaatcaaagtttgcattataattgaaactgaaatttctctgatatctggttatgttagttatagaaaccaaagtcagaaaataattcaatcaaaagccatgcacacatatcatacaatgatatcatttattagtcttatacaaaaaaaataaaaataaataaacatgagtgtatctctgtgtttgcaaaccagaagtttgaataatgggggttaaattcttttccatgtttttgtttctcatgtatgcttagtcacatgttaatttttctgtctaataatttactagtgaacatgccaattgattgttttttttaatccttttctgttgtgaagtatcaaggatgttattgaaaggtacgaagtgcgcacaaatggtgtcgaaaaatcggatgaacagtctcttgagctgcaggtaaatgagttcaatgattttccctttcccccttttaattgttctgcaattaattgtaacatactctgtgatctgcactttggagcttcaaaatgtatctctatagcatccgacggttctgattgtttgaacatagataaaaaaagcattgtcagactcatgtatatgcatgcagcagggtcggcctcttgggctaaatatatataggtggctttgataagtcccaactctagtgggacttcatctggtgtacataaagaattagccaatgaaaaattggaacatagaatcatcatcaaatatcaattattatttacttttaccgttggttgttttccaagcgttcctttttttcatttttaattatgctaaaggtagagtgtgaagtattgtaaccatttgagttacaaattcattgtttttcaaactttagttttaggggcatgcagctgatttaatttcttatgatagttgagttggagacttctattgaatcgatattacagcctcctagagtatcagttttttctccctaattttaataacttattggtttagaaaaaagaaaaggaacgattttacttttttgttttgaaatgagtgattttctgtccaaaatgtagtacgattttactattatgttttgacaatattgattaggggtgggaggccataggcttaagtacatcagttaaaggtaaccaataaccgtagctctcacgcagctgaaaacagagaccaaccgcactcgcatctaggctctcacaccgcaatcacattaaggggaggagctccaccccccttctaacgtcctcgttggcccactctatctacactgactcggtggtagccttttactttctttgaggcggcttcactcaactatcagtattcagtgatcaatatcatgcccatacataacaccgatcatcttaatccaatgtataggtcacccttttgtggaattgggcccagttgcgtggtgaactcaggctttgataccaaatgttaagaactcatgggtagaatttgcccttaaaaaccggcttgtaaggaaaggatgcccttgaactcttaaaccattctataggatatgctctttttccaatgtgggatcttgagtcctaacagacggctaatctttgcccccctctattttaagcccctatgtctctcatctcctctaatttcccttctcctcctctcatcccctcacccacacaccagttcctctctcccccttccatgagatttgaaaccctaaaatcctcctctccccttacgattttcttagtctattggcttgttgcagctgcttctccctccaacccaccctcatatctggcttttgctatggacgtgtggtggactatgtcatttaatggtcaattttgaccccccctggatcacttgagtatgatgaatgttggtggcttgctcctccaaatccatgctatacaaatccttgttggtttctttggacattctccttctaacagcggcggcggcggcgtcgattctgtttttctgttttgggcctagaattgggcttttattttcttttattggttgagcccttgtttctctctttttgactttgtattgggttttatgccatagtagttatgatatttagaacatacaatcccgatctcttggactacaggggtgcaagagttatgccattaatttctgtcataataattatgatatttagaacatgcctctgttccaaaactgtaaaccataaatatttagaacatcaacgcattccacaaatcatgtctccacctacaaatcacactaaacttcacccaaaaccgtaaatatgcctctattccaggtatagccacctcaaaacttgtaatcgaggttcctagaaacttcatacctctagtccaggtaattaaacccataagccacctctagtccagataatgaaatccataagttacctctagtataggtaaaaatgaaacctccacaacatatatagtcacaccaaagtatattgagatgaaactccattaacaaatgctttgaaaaacctaagaaaaaatgctgaatttatcaagggtaaaattgccatatttgtttcaaactagcatgattgtaggagatgcctaaattttagggaggtgctttcatagacttccatttctggaagtggcatacactgttaattcattaaaaattgtggacatacatggtggttggactacaattgaaaaagtagagcatgtggactgccccataatttttcccacttgaattcatcccaatcgtgtcctacatcacatattgtgcgtctctatcaacgcttacatacttgcagagatccaaaccttgaaagttgtctaatctctctaggaaccccaaacctctagacggtgacaaactctcactttgatttcatttgaggcttagcccctacatagccttttttaagtaggctcacagagaaccctaattttgttagctacctaaatcctgaagaaactaggaaatcaaaaatacccagaacgacctcaaaaccccatgaagagaacagaaaacaagcagcacagtacagtccccataaaaatcaccttgcctgaatgtatacaatattcccacacgaaaaaaaactgtaaacacgggttgaagaactccaaatttagagctgctgcacaagactaacatgaatacttaggtcaaaatttcattaaattctttatctctatagaagtagagtggcattgccataaagtccaccaaggataaaaaaaataagtatcttaagctctaatatttgacctagcaatatcatggcctatggaggactttgattatgcgattttaacgtgccatcttccaataaagtaagaagttgggataccctatgtgttaatattgtgctttagatttaatctgttacctcaatttaatcagtacaagttcttattctatttgcctcaatcgaaaattcgaatttctgcgagttgggtttgttgctgttaattaattccctaagtttttcaaaggtattaatgactacgacaaattgtaaatccatgttcttgtatgttcaagtaattccatttcaaaaaaaatattgtaatcctttttttttttttttttttttcccattatgttgtttttttgtttctttacggggctgacgtttaaaagcttttcatgtaacagagacagattaaattctctaaatgatggttccaatatttctcattttagctggagaatgaaaaccacaccaaactcagtacggaacttgaggagaagaaccgccagctgaggtacaaatttattaaatcatcatgaacttgtaagatatatgtggctgtttagctggtattgttgtttgatgtttctgctggaattggactggcttgcaacaggcagatgaaaggtgaggatcttgaagagctggatctggatgagttgctgaagttggaacaactggtggaagcaacccttgtccgtgtgatggaaactaaggtctgccccttgtgtatgcagagtatattttaattgctctgtgttgtgaaaagcaaataaactaacatctaaatgcaaatttcaggaagaactgattatgagtgatattgtggcacttgagaaaaaggttagatgattgatacgtactctgtaaatgaaaacaaatttcagcataaataatcataacaaagtttggctctggttttctcttgcatgcaccaactataatttcagggaactgagctggtagaagccaacaatcagatggtgatgttaagggagagggtaagcaacatatatattgttcttaataaaagatgggttgatccttttttctttttttaaaataaatattttgtattttttgcataacagatggtgatgttatccaaaagaaatactggacctgcgcttatggagccatctgaatctgctacctccaccagctgcaacagtgctctcagtctctctcttgaagatgactgttccgacgacgtcgttttatctctcaaactggggtgagctagctttcttttgctttgctatttgtgtatatatatatctttgctttttaagtttgatgagatcagagaatagaagttttagaattttgaatactcggtcggttgtcgtgcaatacagaacaactaggatattttctgtacttgtcaacttgattctgggtttcaattttaactttaccaaaccaaaaatattggatgttttctgtacttgacaatattttttgtattatagaaatgttatttgaaagtcatcatgagtttcagatatttacagtaatatgtttttggaaaaaagatatatcatttggggctaaccaaagtgttttcacctctttaccacttaccctcccaagcattttaattattcatggtattttatttattcttcaccatttttaataaattttcttttcactttactatgaatttcaggcgtccctaatttccttggttaagaagtggagaaagatgaaagtttactctccttatagctaaataaagaaagttgtggtattgtgagatatcgatgattgtgtgttttaatttgatagcagattgaaaatattgaagggagagtaactttgtgttgtatcaaaacatgtgtattacatgcaacctgtgacatattgctctttgactttgtattgtatttagaactctgtatcaatgcagcctgcgtggagagacttgttaattttgtattgtttgggttatcaatgtcacacaggctaacggtacgcgcgggtcgtagaccgatgtgcttaaagacttagatgtctttcttcattgttttcacgttacaaagctcatttcaagatatcgtgaagtttagtcccacgaatgacattataggtaaaattgtttatttatactattacaactcatgtacaccaaaacttaaaccagacaaaaacaagtgaaataaatttttaatgtaggaaccagaatgtttaagaagaaactcatataattgtctgcgtggattaaatcaacacaagtggcctggggaaagtaacctgagcaagataaagacgaagccatgtgtcatggtgggctcaatcaagtggcttgacagcgcatgacctgtgtcaaagtgccatcctccacacccaaaaaaaaaaaaaaaaattcttctttcctttgtagcctgacacgtaataagaattttaccgacagaaaaaacaaaacttagatatttctgacaaactccttcacaaaaagtcagatgccacattttctcaagtcacatatcaacggtggagattgcattcatcaacgtcatcagcatctgtgcccgtcgatttgctcaggtttcttaaccttgtttccccagaagataatagaagaatatatttttccatataaaggaaaattcataaatcatttcatttcctttcttgaccaacaacaagacagcagcagcagcagccatttctctctctcatttcgaacgcagaccccgaaacccaccaacgaaggtaaacattaaacaaagaggaaaacccagaatttaattcattagggttcttctcctcctcttcttcttcttctttgtgttttgggtggtgggtttttctgggttttgttcatttagatctgggaaccattaaaagtttaaaattgagaagaaacccagaaaatattgtatagtttttgagtgtatgtgaaaagtggttggtttgaatttttttgaagatgatgaggaataagatcaagatcaagaagattgactacttgcctgcaaggcaggtgaccttctcaaaaaggagaagagggctcttcaagaaagctgcagagctatctgttctgtgtgaatctgaggtggcagttgtcatcttttctgctactggcaagcttttagattattcaagctcaaggtaccagcaacataccttataattattttgacttttccttccttgaaaaaataacaatatgtatgatcatgatgtctctgaagtctgatgacttcacttctctctgtgtgggtgagagagagagagagggggagggagattctgagatccagtcatttggttagaaatatatttagaggagagggagagtgcctttatggaaatttcctgtccttccagaaattccatggtttgaactgtgcaaacagacattatttattgtatctaagcttccaacagaaaaaatggagaatccagaagcttccagagagacttttagataggttttttttctgttgtcgtcgaaatatacttttagatagtcatactctgtttcaaaaaaacctagttcgagggtcactaccttgtatatctatccacttaaggaaaataaagtctgcttttgactaagacaacttctggaaagatgtggtgtgtatgatggtgatgttcttttcttgtactacatgcatgcatatgtaaaactgtgtacgaatatatcttgtttagcaaattctgtaatcttaaacccttatacacaagtatatctcgctgcttcaatctcatcctttcccacctatcacatgttggtaatagatattatataatatcttaggtggggcctatacaatgaagaatatcattgtccaagtccaaattaaaattaatgttcccttgtgagagacacgatatgttagaccatcaatcgaatcatacactcaaataatataacagactttttttaaattcatagtttaaccacataaaatgatagtgactatagctagtgagtgagtattcgaccctttacaatatgtatatacatctatatgtatatatgttatccttcgaacattcttcccacttaaactaatgttctcttgtgagagacacgatatattagaccatcaatcgaatcatacactcaaacaagaaagcagattaacaattctagattcgtagtctgatcacaggaaatgttagataatctagctagtaagagagtatttaactctttaccatatatatacatatctatatgtatcttatccatcgaagttaagatatttgtgggttgtgtttgaaaaggacctaaaagacaaaatgcccgtacattaatgtggttgcaaacattgaattatatgtttgtaattttcttactaggaataaagtggtatgatgataacttgagaattcatcatagaagccttgaagactttcagcaggaatatgcatgcatgctttcaactagggcaatgggtggtggagctatgaccataacatgtttttgccatggaataagaattagatgtgcatgcatgagaggaaggaaagagagggagggtgagaatgaaatgttaactatggaataagaatttaaataaggtcacgcatcctttcatttatattttttaaaaaacncccnaactagaaataccncctccaaagtggtcttctttaacccnaaaatatactcttaaggtggtcttatatggcgatactatttgcaagagagtgaatgtcgttaaacatttttttttttttttttttaaaaaaaaaagctttgatacaaatggaggtgtcagttaaaaataacaaattggagacgacatttaattgtttatgtctgtatgcaatttctaatcagtttcctttaaaagttatttttctaggtttcctaattgacatgttatatgcaaagttattttatatatttttttaaaggttttctaattgtatttatattcaagtaccagagtacttgcaaccaaaacaaaaaccgtgaaatgaatattttactattacattatttttcaaacttctcgaagtttcctagtagagcaatattggtccgaattctccaagtttggaaaagtattgctcttggatattatactatattttcaaagatttatttagtattgtgtcgaaatatttatataggaactccaaagtcatgtgtgttcatgagacttgcccaatattattattaagttttgagatgtttcattcatatttgtattttgaaactcttaagggcttgtttagcgaggccatgttataacatgttggagtccttagccacttacaccaagttttcttcccattttagtcggtcaccacttcatagtacatcttcttcccatgtcttagagtccaccactccctatgttgtcacttggttgtttaatgttttacatgattccaagttttctactagattttcatgtcctattagcctagctttggggagtctagatccccttgttccagtgggataatactattcactacaagggtgactttttaagaaagtaacaatcctttagaccgataaggatgataaatcaagctcataatgttggtgctatgactacctatgcaagtaccaaaaatgggtcaattaaggaggtagaagtcgtatgatgattttagtctttctagatgatgcgaacaaaattcaagagccgtgcgtcctgtaaaaagctagatgaagatggctaaaacacttgttcgagacactactttggacctttaatattttcttcaattctgtaatttggttgaaaatacttacattgtgggcgtctacttgcattaatgacgcatcgtgacaatcggatgactaaagatttagcttaaaacacacaactttggaatccaccatgaagtagtgaggaaaccttaagtttattgataatctgaaataacataacctaaccctagccacaggttaaaaattctttaaataccctagaagccctactaataaacaaggaattaaaataggaaaaattacaaagttttcaaaaacttataagacataaaaactggattttgggttgttaacgatctcggatgcctgaaaaagctgttctcttcgaatcaatgggtcatgggcccaaacttgcagctttctaagtgtgccctttagcgcgcctcttcaactcttcttcaattgccttcgacatcttttctacatgaattaagtatgttcaaagaagagagttcttgtctaagatgtcttaaccacgttttagtatttcgatctctaagggcgtattgaggaatggagatgatgatgtcgggacattgtagaaagctggtgtcgggatgatggttgtggatttattcacctagtagtgacttttgaaatgttttgcgcaaaatgagtggctttctttacccgttttctattctgcctgatggcttgtgaaaattttggtacaaacactaacttaactagtatgtggtttagtaatcaagcaagcaagatcttaggcagtagaagttgcctaagattggcgagttttggttttcggatgattgccaaaccacgacaagttggtttgtttaatggttctttctagcttggtatgaggtcactctggggagcttgagttttggtttaagatttaacaatcaatctattagaagatacataagtgtaacaatcaatctattaatattcataggcgcgaaagatttttccaatgtattgtttatcaatcttttccaccaacatcgaatgaccccatacaaggaaaaatgaattcgaattcaagtaaatctattattttttttgtcaaatggtgatatttcatttcgaaacctcgtcaaaaatggggagggcacaaaaactacaaaacaaagcaacctagaaaggaagctcactaatacaatgcgtctgatctctattagagatacgatatataagttggtctctaaagatagtcaccctagcattttccatatataaatgccttgtgttgtaaactatctcagttgcttgttgggttggtcttttatatatatatatatatatagggacattctccaataaatattcattaaatatgttggtagtctcttagggaccgtatatatatatatatatatataggttttctataaaatatctatgaaggaatttgatttgacgttttgatttgcataaccaaaccatgcgataaagccagcagcaaatagcttcacgttctcattatgcatataaaacaagtatttatttcatttcaaaatctccaaaatcatagttgtgatgatggtcttgaatgaaacccttttctgatgctatcaaggaacagtactaatggtacaaaaatatccaaatctgtaacatatatattttttaatttgtgggggaagggagggctaaattagttaatttgcttttcactgttgaatttacttttttaataaatcatagttagattattgcatgcatgtccaaagtggttggttagattattgcatgctattttttgtaaaaacatctaatggaattattctttgtacttgttttctcttgagatgattgggttattctatggtgtagcccttttttggatctccaatatataactggaaaaattctgggtctgcagaaaattggaaatccaatcctgggtttgcttgtattttgctttataattgaaaatgaaattatgggtttgcattataattggaaaccaaattccaacacaaaaagttctgaattttttgcagtggggggtaagttgtagatcactctgattgctggttatgttagttagagacacttaagtatgaaaataattcaattttttggcaagatgccatcatgctttatggcttagcgttgaagttatgccagtgataaattgcagattatcaaaaccctgaattagacattagtgtgtgtgtcttggtgtttgcaaaccagaagtttgaataatggggttaaattcttttccatttttttgtttctcatgtatgttttgtcacatgttagttcttcaatgtcttaacaatttactagtgaacatgccgattgatttttatttttttttgttttttgttatgaagtaccaaggatgttattgaaaggtacaacgcagacataaatggtgtcgaaaaattgaacaatcaagagattgagctgcaggtaaatgagttcaatggttaccttttccgtttcccccttttaaattgttctgcaattaatggtaacatactttgtaatctgcactttggagcttcaaaatatctctctatgtagcatctgaaggttctgattgtttgaacataaataaaaacattatttcctgttacatgccagacttatgtacatatgcatgcagcaggggtcggcctcttgggctaaatatatgtagctaggtggctttgataagtccatttctagtgggacttatgaatttggtctacagatggaaggaaatagtagtcgtgggccaagtcctggtgttgttttgagaattagccaatgaaatagattcatcagcaactatcaatcaatcatattttttttcagcaaaaaataccctgaacgatcgcaaaacccacaaaaataaataataataaaataataataataataatttcccttgttgccccagcaccagaagactctaaacacaaaaatttggagcagcttagcacaagaataacatgaatacttgggtcaaaatttcattaaactccttatctctatagaatttgagtaatcaggaaagaaactctgttttggtgctagagtagtggttggttggggagtaaaaatgttgaagtttttgccctaacaatatggatgagtggtatcctgccatcttccaataaagtaagattcccctacgtgttaatattgtgctttagatttaatctgtcatctcaatttaatctgtcatctgttctttttccatttgcctttatctaagttgggtttgttgctattaatgaaatccctaagtttttctaaggtattaatgactaggacaaattgtaaatccatgttcttgtatgtccaagtaatcatttgattccattttcaaaaatatattgtaatcctttttgtttttttttccccttatatttttcgatgctgacgtttataagcttttcatttgacagagacagattaaattatgtaatggttcccatatttatcattttagctggagaatgaaaaccacatcaaactgagtaaggaactcgaggagaagagccgccagctgaggtacaaaattcatgatcttgtaagatatatgtggctgtttagctggtattgttgtttgatgtatctgctgaaattggacgggcttgcaacaggcagatgaaaggtgaggatcttgaagggctgaatctggatgagttgttgaagttggaacaactggtggaagcaagccttggccgtgtcatggaaactaaggtctgccctagctattttattttctagtaaccagtgccctagcttcttctttttctttttctttttttttcttttctggatgattcttgactgaagacataagcaatgaaccgttagaataatataaagtaggtctttgtttgaagaagtatcgtctcagtaaatagggtatatttttattgctgtattttgtgaaaagcataggtgtattctgagttgtcactaagctttattttctgaaaactaacatctaaatgctgttcctttcttttccaggaagagctgattaagagtgagattatggcacttgaaagaaaggttagatgattgatacgtacagatctgtaaatggaaacaaacgtaaaataatcacgacttaaaaacatatataaattaacatcaataatcataaccaagtttggctttggttctctctttcattcactgactataatttcagggaactgagctagttgaagctaacaaccagctaaggcagacggtaagcaactaccacaatcatgtacatacttcttcttactttttcttctcctctttttgttttttgagaggaagagtgctcttaataaaagattatgggttgatctttttttctcttgtaattttttgcatagcagatggtgatgttatccggaggaaatactggacctgcgcttatggatccggagaggttgaataataatattgaaggtggaggagaagaagaaggcatgtcagctgaatctgctatctccaccacctgcaacagtgctgtcagtctctctcttgaagatgactcctccgatgaggtcactttgtctctcaaactggggtgagctacttttgctatttgctatttatatgcttttaagtttcatctgagaagacatgcttgatcattgctttatttgatggtacgcgggcgtcgtgcaataagagggaggaggatatttcccttcccttgtgttgtgagggttttgcttagatttatttctgtacttgtcaactttatcttgggtttcattttttaactctaccaaaccaaaatatttggatttttagaaaagaaaaactatgaattgcttatcccgccgacattttttttaaatatagaagtattgacaattctcagaagtcatcatgaatttcagataaattgtacaaaaacaattgcgcgatgatttatttcccagcaacttcaatattttcagtaatacagtataagtgctcatttgggcctaactgttttcacctatttactacaccggagcttctacaatgaggagtttagagtgaggagcttatttgaggggtttaatcccactatcaaactatttaaaatgtttaagttcattgattttctcccatttgttgatggtgggattgaactcttcaaataagttcctcactctaaacacttcattagagcctttccgtctacatactaaccctcccaattcgattgaagatgttttaggggtttgtagatcaagtttcttttgaggtttcatttttatgtatttgggccatttcattttttgagtttatttaaattgaaagaagtaagtgactttcactgaaatttcacatttttttaaattatattttgttatagaaaataatagatggcttatgtgaattaacgtcatcgttattgcatagtatggcaatctaaataaaattaatttaaacatttttttgtatgaaaataaaaataattaactttcccgcattgttttatgaaaattacagaaagcttcagttgaagaatcccaacactgaaaggggctgaaaatgtaggggagaatttattccacacccactatgacagcgtaacctgcataatacaagttaaaacatccccacaaacaaatgttggtgcacagttgcattcatggaattttatttattcttcacattttaaataaatttactaatcattttgtacgtattttctctgaatttcaggcgttaagaagtgaagaaagatgaaagtttactctccttataactaaataaataaagttgaggtattgtaagatatcagtaattgtgtctcttaactcgacataacattgaaaaatatcaaagggagggtgactgtgtgttgtatctaaacctatgcgttacatgcaagcttgaaattgctctgactttgtattgtatttaaaactctgtattaatgcagcttgcatggtgagttatgttgattttgaatttgcttgggttatcattgtcacagcgagacacgtactgtttgcgtggattaaatggataaaagtgaactggggggaaggaacctgagcaagatatagacagccacgtgtcgcatggtgggcccaatcaagtgacatgacagcgaatgacctgtctccatccgactccacataaaaataaaatttattcatttttttgtaaccctacacgtagtaagaattaaaccgacaacaaactaaacttaagatacttctgaacgaaaaaacataacttagatatggacaaatcgatcacaaaaagtaagatgctacattttgtgctgacgcattacaagatcaacggtggagattgcatgcatcaacggcaaggtgcatctgtccccgtcgatttgctcaggtttcttacattgttttcccagaagagaccgaaagtaaagcagaggataatagaaatagatgttttccataaaaaggaaaatttcctttcttgaacaaccagccaccaccagcagccgccctttatctctctagttctcttagtttttctggacagaccaaaacttcagaccgggctgaaaccccgacaaaggtaaacattaaaaacagagaggaaaacccagagattaattaaattaattaggcttcttcttcttcttcttcttcctcttcttctttttgttgtgaacttgtgacctattttggttggtttcttcttcttcttctttttgttgtgaacttgtgacctattttggttggtgggttttttctgggttttgttcatttagatctggggaccattaaaaggttaaattttgagaaggaacccagaaaatattgtgtagtttttgagtgtatgaagataataatatatgtgaaaagtggttggtttgaatttttttgaagggaaacggtgaaaatgatgagggagaagatcaagatcaagaagattgactacctgccagcaaggcaggtgaccttctcaaagagaagaagagggctcttcaagaaagctgcagagctatctgttctgtgtgaatctgaggtggctgtcatcatcttctctgctactgacaagctctttgattattcaagctcaaggtaccagcacccaccactaaataccttataattcttttgactctttccttccttgcaatgataaccaatatgtatgatcatgatgtctgaagtttgacttgacttgtgtgtgtgtgtgtgtgcgagagagagagagagagagagagagagagagagagagagaatctgagattcagtcattttggtagaaatatatttagaggagagggtgccttttatggaaattacctgtccttcctgaaattccatggtttgaactctgcaaacagacattatttattgtgtcaaaggcaaatagaagcttctagagagagagagagagagaaaatcttttagatagttatactctatttcaaaggcaactagttcgagggttaccaccttgtatattttttattaagacaacttctagctaggtgtcgcagtttgagaaacattatatgatgatgttcttttcttgtactgcatgcgcaatcaattttgtttaccaagcaaaactgtgtatatcttttttggacaaagtgtatataaagaagctgtaatcttatcctctcccacctatcacatattggtggtagatattatatatataaaatcttaggtggggcttatacaatgaagaatatccttgtttcaatccaaggtcataaaattaatggttctcttgtgagagacacggtatgttagaccatcaattaaatcatacactcaaacaagaatatagatcaccaaatttagattcgtagtttgaccgcataaaatgatagatagtctagctagtaagagagtattttactctttcccataccatatatacatatgtataccatatgtatatatcttatccttaaaacttaagataatggaaggtgtctttcattctcccctcccaaactaaaaaaatgccagtaaattaatgtggttacaaacattgaattatatgtttgtagttttcttactagaaataatgtgatatgatgaagccctgaagactttcagcaggaatatccatgcatgctttcaaagtagggcaatgggtggtggagctatgaccataacatatttttgccatgaatgcatgaaagattttgttaaaacctagtttcatactttagatgtgcatgcatgagagggagggtgagaatgaaatgttaactatggaataagaatttaaataaggtcatgcttcctttcagttaaaaagaattccgttatctagatgttcctattttactattaataacatgcgtcaaatgaaggaaacagattaaaagacaatttgaaaatgcaaatgcaaattaagaacaaaattaaaagactttttaactgtttatgtcagtatgcaatttcttatcaatttctattgaaaatttcatgtaaaatttatttttctaggtttccgggcataatttggcacgttatatgcaaagctgctagcttgtctagtttgtctattttgtattttttttcaagtttttctattaaagctctcgatattgcagtccatgttttcatttttgcaaccagaacaaaacataaaacgattttactcaaatcaatatacattgaaaaactagaggagagtaaaagtgtttttctttagccttagattttactttacattatttttcaaacttttccaattttccaattagagcaatattggtccaaattttccaattataaatagggcttgttaagcgaggtcatgttaacactaaacatgattctaagttttctataagggtgggtatggtccgattctcaccccaaattagaatcagaactattcatctagtttggttcaatttggttttgacgaaaaaattctccggtctagtgtggtacgatttgcccatgagtaggttcatgtcacaattttggatcggaatccctttttaaaattttttttttttgggcttaaaaattcacaaattgcccaatttcatgcaaagaaaggtgattgggcctagaaaagagaggtgatttgtagttgggcttggggaaatattaattatgggcttagaaattttgcattgggcttaaattttttattaaaaacttaaaaaataaatagtcggttcggtttggtttagtccgattttttcactacaagaatcgaaatcaaaatcaaaccaaactggtttggtttttggtcaactcggttattttttgccccaatcagctctgtttatgcccaccggtaccctagttttctactcgattttcatgtcctgttagcctagctttggggatgttgcagtagggcttgattaagttattcttcaagaatcaatggtttaaaagagtctattaatatatctaccagggaattggcctccaagtgatgtgtgaagtttccgacgtgtttaatgatctttctagggtgcatctggcttctgaagctctaaagtgtttgagaagaacaagattagttaagaatgaaatgttatagtctaaggtgtgttaactacattttagttcttcggtgtctatggccgtactgaggaatggagacgatcacgtcctgacataggagaaagatgacgttgggccgatggtcatggatatattcacctaggtagagactcttgatatttttggcgcaagccagtagaagtttgaacttgatttgcaccataaaattaataacaattcacttatatgtgatatcgaaggtgtatgaatatgtgtcgttcggtagtattattggcataggcctacgtagagttgcgtgaattgtcagatctatcactaccctgcaagaaaaacttggatctgattcgtatttgcatggtagctggcgctgaagaattcaggataaaaagaagccaatcaaaaacacccactcactaaaaagaggccaaccaaagataacaatattcaaagcatatttcaatgagctccagagttgatgtgagaggaactgtctgtgactaagatttttgaggcccacttattagccgacttgattttctcacgtcggtttgccttgtgttctaaaatatctcagctgcttgggttggtcttatatataataattccataaaatatctatgaaagaattagagttgacgttttgatttgcatatgtaatcaaaccaagcaataaaaccagcagcaaatagcttaacgttctcattatgcatatcaaataaaattatatatatatatatatatatatgttagtgtatatctgtgtttgcaaaccagaagtttgaataatgggggttaaattcttttccctttttttttgtttctcacgtatgcttagtcacacgttaatttttctatgtctaataatttactagcgaacatgccaattgattgattgttttttttccttttgtgttgtgaagtaccgaggatgttattgaaaggtacaaagcgcacacaaatgatctcgaaaaatcgaacaaacagtttcttgagctgcaagtaaatgagttccatgaccttttctctttccccttttaaattgttctgcaattaattgtaacattactctgtgatctgcactttggagattcaaaatgtatctctatagcaactgacggttctgattattgtttgaacataaaataaaataaaacattatcctgttacatgccagacttatgtatatgcatgcagcagggtcggcatcttgggctaaatatatatgtaggtggctttgattagtccatttctagtggacttaatttggtctacataacaattagccaatgaaaaaattgggacatggattcatcatcaactatcaattattatttacttttattgttagttgttttccaagtgttcaaaggagagggtaaattattttataggtttttacaaataagtccattatatagaggctacttgaagataaaaacccaaaattagtcacttgtcattttctaattggtcttagaaaatttgaacacactaataaccaatcagaaaatgacaagtggttagttttggatttttctctccaaatagcctctatatgcgtttttgtatagtatatcatgttagaatggacctatttgtgaaaagtctattattgaattgtttttcaagctttaggggatttaatttcttatggttttttcagaactccagatagttgagttggagacttctatgtgtccctctcactttggttgcaattgaggcttagcccctaaataacctctttcacaggctcacatataaccctaattttgttagctacctaaatcctgacaaaactaggaaatcaataaatacccagaacgactaggacaaattgtaaatccatgttcttgtatgttccagtgattcatttgattccattttcaaaaatatattgtaatcatttttgttcttctcgttatgttgtttctttgttttttttcagggctgacgtttataagctttttatgtaacagattagattatgtaatggttccaatatttctcattttagctggagaatgaaaaccacatcaaactgagtaaggaactcgaggagaagagccgccagctgaggtacaaattaattaaatcatcatgaacttgtaagatatgtgtggctgtttagctggtattattgcttgaattatgtgcgggaattggactggcttgcaacaggcagatgaaaggtgaggatcttcaagggctgaatatggatgagctgctgaagttagaacaactggtggaagcaagccttggccgtgtgatagaaactaaggtctgccctagctaatttcttttctagtaaggatggtgcaaggcttcactctctgtattgtggtttaatgatcgaacccgtttattataatttaggaatccttgttgcagaatagtcaaatggtgttagtacaagcgataatctaaattattagagggggggttctcacacacactcacacgactaagataccaagggggttcgaacctgagacctttggtctgcaaattaaagtcattttccattgggttagaccccgttagcttagttaaatggtgcaagacattgcatttgtgttccaataagtaaacaacagcatctctacaaaacactgaaatcattaccatttaatgaaatggtataatagtatccaatttttttgccggatgattcttgaatgaaaacctaaatgatgaactgttcgaataatataaagtaggtcccacaattgtcctttgtttgaagaagttgtttgtgcatagagagtatatgttctgggttgtcactataaagtaacatctaaatgcaaatgttccattccctttcaggaagagctgattatgagtgagattatggcactggagaaaaaggttagatgattgatacgtactctgtaaatgaaaacaaatttcaactatttacaataatcaggactttaacatcaataatcataaccaagtttggctctggttttctcttccactcacaccgactataatttcagggagctgagctggtagaagccaacaaccagttaaggcagaaggtaagcaactaccgcaatcatgtacatatttcttcttctttttatctttatctttttttattttgtattttttgcatagcagatggcgatgttatccggaggaaatactggacctgcgtttgtggagccggagacgttgattactaatgttggaggtggaggagaagaagacggcatgtcatctgaatctgccataattgccacctccaccagctgcaacagtgctcacagtctctctcttgaagatgactgctccgatgtcactttatctctcaaactggggtgagctacttttgctttgctacgtatatttatttatttatttatatgatttttttaaagtttgatcagagaagacatgcttgctttatttgatgatactatgaagaataaattaaaagttaaaataatcaaaatatttggattattagattgaagatgtttattttgaggtttcatgtttgtgtctttgggctatttcatttttgtgtttatttaaattgaaacaaagtcagtgactttcatcgaactctctcacatttttttatatatattttgtttaaaatatttatataaaaaaatgtgaatttcgtagtatggcagtccaaataaaattattttaaacatttttatataactaataataattaacttgctacagaaatcttcagttacgaaatcccaacactgaaagggctattgaaaacatgcgggagaaaatatattccactctctctagactaaaacaacgtaacctgaataaggcatacatggtatttcatttattcttcacctttttaataaatttgcaaatcactttgtattttctctgaatttcaggcttccctagtttcttggttaagaagtggacaaggaagaaagtttactcactttatagctaaataaacaaaatcgagataatgtgagacgcatcagtagtgatcgtgtgtcttaactcgatatgacattgaagggagagtaactttctgtgtatctaaacctatggtattacatgcagcctgacatactgctcttcgactttgtattgtatgtattaatgcagcctgcacggagactactagatgttgtgttgattgggttatcagtgtcacagcacatgtcaaccttataaggtcgtgtgttttttatttttatttttttttatacacagaggaaaacttcaagcaaaaaatcacgagaacacgtaggctccaacatgttaaaatagaaagggatcgaatggttcagaggaagccccgcctcccaaagcttggaaggatttagcccatgtcccaaactagcaacatcatcagtcgtgaaataatgtaaagaaactcatgaacctaaatgaactctactagtaccactacttgaagaaatgtcgtaacagtcatacccacaattgaatccgtaacatatttggggaaaatataatttccagcgatctatctgcgcataaaagcaaataaataaacgaaaacaacaacattgtcaaagaacaattaattaattaaacatattagtccacatggcaaaatatgaatgagtccaggccacatcattatttaacggagaacattatggtcaaattaactaatgtatgaaagtgatacaaaaataaacattatgtatgggactgagacgagaaaaaacttgatgtatcgaaggctgaaacttgataaacctgaacatagtaaaagaataattctacttttattatatttgtaaaccatattttcataccatcttatataacagttaaagtgaacaaccacatccattaaaattatttcactttttttttcttttataatttattctagtatctcgtagtaaaatgaaaattacccaaatatttattactcacttcttattaccagcgaaaagtaaaagaaaaaagaaaaaagaaaagagaacaatggcttcgatgcgatttcatcgaatgaggacgttcacattataacttccatacgtcctttctttcccgtaaaaattagatcggattcatagttgtcacctccattgacgacagtgagcttctcttctcttctctgctgccctacacaactgaaaacgcaatgaatatgcaggtttagggttttccaatcaaaactcaaacgaacccatgaagcttcacaaggactactatggctactactatcccaaccagtctcagagagccgctttagctggccttttggtggtcctctttccggccttcttcccaaatctcttcggcccattaggccgtgcctctccttccttcttctcagtaagtcccttcccaaacaaccattctcacagtttgatgttgttgttaatggaaatgcttaattcttaatgaaactgcaatgggtattttgcaggagtggaatgctccaaaggccagacatttgcatcttctaaatccagctttactttcccaatctgtcagttccttcttttcttttcctttgatttgtgatttgttttcagaattggtgtaaacaattgatctatttacatatattaacatattctttttgaatttgtttagtctgttgagcaacagagcaagctatggtctccactgcccaaccaaggatggaaacattgtcttgaagagcctaaaagtctatcatgtgagtgctaaagatcgggccacattttatgtgacattgaattgattgttagctgatgaaattttattcattttcttgcagtgacaccaaaaggatctactgggtatattcaggtgttccttgatggaggcttgaaccagcagaaaatgggggtgggtttttaaattttataaggaaattctttctaagcaatgtatcttgtagaaaatgaagttatacaagaatgcccgtttacttgatttgattacttgttgtgatactatagccaattgggtgtccaaaatgagattgtctaatttactttatttggttcttcaaaaaaaaaaaaaaaaaaaaaagatttgtgatgcagttgctgttgccaaaattttgaatgccacattggttgttccacaccttgaagttaatcctgtgtggcaagattcgaggtatatcgtgagtatgctaacctggaacctttcctacaatttattatttctgtccaaagaggagccattgatttgaaaccccatgtgcttttgcagttcctttgaggaaatttttgatttggatcactttattgaagtcttacgcgatgaagtttctatagttaaagacctgcctagtgaattttcttggagcactagggagtattatgctactggcattcggattactagaataaaaacagcacctgtccatgcttcttctgattggtatctggaaaatgtcttgcccatattgcagaggtcagtactgcatcgagcctttcttaattgagcgtaaagttcttgcttttttcaatcacttgagcctgttcttgcagttttctgttttatgtgaagttggtatcgtaaacttttgtccctatgttgtcttagtgtcatatactaacttcacatgcaaaaacagaaatctgcaagataaactcttgtccctatgctgtcttagtatcagatactcatgtccatattctgtctgtccttcaatttagtctcctgcctgcatggattgtgcagaggtttcatttcttaaaattgtgattgtgatattgcttgtgttagaaacttgtataatctgtcccttttttttccttcttttttttccagtaaatagcagtgataagttatcataacaagaaatatttaggaatgataatattatgatgctatagacatagctttgggaaataagttgtttggttaatgtgtatagcatatagttagaacttagaattagtgcatcacagacgtatttgtgtctttacatgtcaagccaccagcttttcaagttagattatgtattgcatctctagttttaggattttaatggttatctagttttagactgaaaatatccttttgggtttatttccagttatcttctctgggtacaacttgagttatcaatcttaagtatgtgcatatatgtcttgctagaatcatgacaataggtttcttttttagaactaaatttgaaattaatatcttattatgattggaaggtgtttcactgatgcagatacggagttgctgctatttccccattttctcatcgtttgacttttgagaacttgcctaaaaacatccagcgcctccgttgtaaagtcaactttaaagctttagcctttgttcctcatatcagggaattgggagaaacccttgttaatcgtctccgctaccctcctaatagaaaccaagacgcagcaagtgactcacaggacggaacaaatcaaattgagaaacagggagctgggaaatttgttgttttgcatcttcgctttgataaagtatgtttgtaattttctttagatccatgtcatgtttgatcatgcaataattttttaagaatcttaaagcttttccgacgaaccctaattatatatgcaaaattagtatttctaatactctttgcctgtacatactcataggatatggctgctcattcagcctgtgattttggtggtggtaaagctgagaaactcgcccttgcaaaatatcggcaagtaatttggcagggtagggttctaaaatctcagtttacagatgaggagttgagaaatcaggggcgttgcccattgactcctgaagagattgggttgctgctagcagctttgggcttcaacaatagtacccggctctatcttgcttctcacaaggtatttcttaaaagcagaatgtgtatactgttattcatgagctaacagctaggggcttgtttgttgctggtttgtttttttatttttgttttttgttttttgtttttcttaaaacagctttagttaggtattctccttcttccctctcattttcatcccctctcttctttctctcatagctttccctctttctcttcaattaaaacaaaagcaaacacattgacaaataagccccaagtgtttcatgtgattccttaggttcctttttagtaaatgttgataccgtgtgttcagttatgggtttaaattggataaacgagtcactattttccttgtttaggtttatggcggagaagcaaggatctcaactttgagaagattatttccactaatggaagacaagaagagtcttgcctctgcagaggaaagggccaaagttgaaggcaaggcttctctattagccgcagttgactattatgtgagcatgcaaagtgacatcttcatctctgcttctcccgggaatatgcacaatgcactggtaagagtatcaagttcaatactcggtagatatgtgtcttctttaagatggtgggggaataaaccagataatctacttcatccaatctgtggttgtagtgaaatatgtgaatataggaattccctaaatagcctattattataaaatcaggtatttcagcagaagaattatttccttgaaattttgtgaaaattctaagtggacaggattaaaatttgtatcctcaggtcagtcatcgcgcttacatgaacttgaagactattagaccaagcatgtcattgttgggaaagcttttcgtgaataagagcatggaatggtcagagttccaacatgcagttctggatgggcataaaagtagacaagggcagattaggttgaggaaggagaagcagtccatttatacataccctgctcctgattgcatgtgtcaagcttaatagaaatcgatttcggaggctgtggttttgctttgaacgtctgtcattactggcaagtaatttttgtaggtcagctacgtatgagtattggagttatccagtctcattgctgtataggcttcatctgagctcgcaagtgtatagtctgctttcaatgcatatcagcctgtaggacaagcattgattctcaccaacagaatgacaaacccacacttttcctctttttcttttttggtcacaatgcaaggtaatatgatgaatttaagcttggggtaatcccaagtgcttgactatttcagtttaatatggtggatttaagcttcaggcatttaatccaagctgttgtatgcctcctcttctccctccccccttcaaagaatttttacctccttagcaaccaaaagaaaaatgcaatacttacgaggcctctgcttgacacaaaccaactctcataggactcgtttggttcacggaaaggatttgatggaaaataatttttcatgtctttttcaaatgtataggaaacgaaaaattcctttcccatatttgatttgcctaggaatgtaaaaaaaaaaaaaattgtttgatttttaaattatacccatattagataaaattaaaaaaagaatgcatttaatgatatatcgtaattttaaattgttaatggggacaaaataatcatgaaaaatgtgcatttaattaggggtgggcatgagttgggttgggtcaattttagtcataactcgtaacccaacccgatatatggcggtttatgatttcttgaattcaaaacccgtacattattagaccaactcaatccaatccaaacttttggtaatcgagttgggctgttcggttcggttaactcaaaattaagcatatttttaagttttgatttcaactcataactcaacctatacatagcggtttatgatttttgaaccaaaaacccgctcatttttaagccacccaacccactatgatcgagttaagagattcagttgggttgactcaaaattgtgcccacccctacatttaatgttggcctaagttttccatgggaagaaaaaatgaaactcaacgggggtggagggtttcattttcccccctatttttcttattttttcatataccaggcaaccatttcccatgtctggacttaccaaacatgagagatccccatttcccactaaccaaagcaaaggccattccgcttggatgaggccacagtaattacacgttttaacatgtgacatctaatcattgctccataatgaaacgatctttccaaatcaagtaaagaggcgaaatttgcaatcacacatcaaacatatgcgttaaaaagttttctacacctcccttttcttttctttttttgagcttttcagttttcactttcagggatgggatgggatgggagaaactggtcgtttcagattgacccacaaatttccatttcaacaaattatcaagcaatcgtagaagacaatgtaatgccagcttcaaaaactcagcggcatgcatttttacacgtcaatcaataactgtgttacactataacgactaacgacgtagcctttatggtcctgctattcagtttgcatggctctaggaactgcaggcaattaaccatctttcacatctgtgccaacaggaggagaccctccaccatttcggtgggcagtgagttcttttgattctgcagcaagagatcgagccatgtcttcttcctcaattggaagtgttcgctttgagccctccaaagcaacaccaaggataatattctcttctaagggaggtctagctatggatggtgatgaaacaggtttctcaccatcatgctttgcttgtaaaggtgccagaggaacatctgctttttcactaccagtctcaggcgaagcaacaagggacttcttagatgtcatctttggtggagacgcatctttggaatcaactcctgttgtctctttccctgcattttttgtactctcattcttggactgcagcatttcagagttgcttcgaactgaaccatcagatgccgaattccgtgtttggggttcagatgttggcatttccgaggtcttggagtttgtgcttgagttggaacttgaagttgctgcaactttattgtcagtttgtgcatgagttagtgttgccccagctttagcatctgatcccttggagtcggatgttgaggaagcttcagcctgggcctgtttgtctccatttgtacgtgttgggcgacttgaagctttacttttgtcatcactactgattttataagatggttcaataagtagatatggacgattattggatgccctagaatgagtgaaaatggtgtcagcaaatggcacattttccaagtcagcctcactgtaatatttctggactgtgcgaatgggtgtggcaagccgggcacgatgatggctgacaactctgagaagatccaaaagtattgcctcctgttttatttcatcaatagagtttatccgagataaatattcaaccgtttctcaaagactataattaacattgagaaagtaaacagaattgtgatatccataaacaactacaaagttcaggcatacagacacttggaaaccttagtaacagtaaacaacacaaattatatagtccaaatcaaaagtctggtataaaaaaggtaaaactgattctttgagtaacttgtgaaccaatcttaaaatggcaatcagtcttcataaattgctcaacttttccatccaatgaaaaaggaaatgccaagaaaagacaaaattatcttgttcaacatgaacaatctataagccagactaaaggttcatcaagaaacaatgtattggaaaggaagagtgaagaatagaatatccaaacaaggttatggcattaccattatacaaaataaatctgagcattaagtcccaacaatcatgcaactgtaatcaatatggcagagaactagggtgagtgagtgtgacttttacatttattatatgcagcatctctggaaaactcatattggcatctaagtcaaagatgatcaaaatttcatactattttcagaatctattatatcatcttgcaggttacatgatttcataaagcttatactaataatgtagtcttaccttaacacagagatactcctcaaaatgtgatgttttaacaaagcaggataccaaaatctgtgacaaaaatagaccgcttatgagaacaaccattcaatacaaaatttaatagaaaggaagaagaaaacaaaaaagagggacgtattaaatggagattgtatatccataggaaaatcaaaagatgagtttctttattctatgaagaccttgtgaatgaaagtgaagaattgcgagaccataaaaacatacacaacagtttccatcatatatttcgaaagtgaaaaaaacttattacttttaaagtaatagaatgtttttacaagggacttgaagaaggtttttcttaaaatttgtttgccaaatggaacatctattgaaacattataaacctacttataaaaaagtaatcatactaacttcaaaagtgaaagcacaaaagccataccataagggcctgattatctggattaatattgtccagaaaaactctcctgtgcaacctttgctgctctacttgagaatttttggccaaaaccttgcgcatatcagctacaatagtctgcagatagtaagggataaaccaggttctcatatgaaatgatataaaaaacacaaagtccttggagcacgagtttcgcaagccagtgaactgaaatttctactcacgttaattttaatgacatccaagtgacttatggctaggtgggtcttaatgcgccaatgagtcttctggctaagattcctgacaacgtttacagtgaacttgtgatttggaatgtgaactgcttcgcgatcatcgcctcttataattgttggtgaccaccaacccacatgctggtacaataacaaatttatcaacgacaaaaaaaaaatgtccatattgcttataaaaaattcacttccagtatttgttttgcaaaaggaaccaaattgttaaagccctattatgtttaaggtggaagccatcaaaggtattcatgggtaggcattatgttttttccttctaaccgctgttgcacttgatgtattagcaaagatatatacaaacctcaacagtaccagaaacttcatagccttcaatcttagtctgaatccattcattaacaacaaatggtcgtgtggcatggatcattacacttgaaaggaagtttgtgaatatctgagttcagacaatattaaggtacatgtagtcagaaaaccttaaaggaaattatgtcttaacagaatttgaaagtatacacaacacatggacaatcagaatttgttatgatcaataaactgcagccttttcaatttacaatatcacatgaagaaagatctgtacctcgcgaccagcaagggtgagcaacactgtgccaagaccaccagctgttagccatttctgggttgagaatcccagcagctccatgaacaatgatactgcagcaacccaaactgcagaataaacagccttgccagcgaaattgaagcccatctgccccaacaagagacaattatgtaaggatgcaataatcttatatgctacaagtgccctcatgacagctacattattgcataatgacataatatctcacaataacagaaacttcgttggaaaaggaaaactatgctacaatctatttcaatgaggtcaaaataagcagtaaagaagctaactagatgaggcccattcatggaaacattaatcaggactggttacatttagttcattttattttaattatttgcgtgagtactaatgacaattttataacagaagtataaaggcgaaagaatgcatacaaagtaatgaaaatataatatgtgaactagtaacagaaacatacatttctcgaatcacttgggtcacttgtctccgtaaagaacttttgtgcttgttgattcaagctgcaaagaaaaagtgtaacaatagaaaatcaaaatttcaaccccaatgcagtgactacaggatcaatgtattgaaacagtgattacaggacacgtgagggcaaaaagaaaatcatatgtgcatagagagcatagcaccaaaaggaaaaactataccgaaaaaattgaaatgaactacgtcctggcagaagaaataaacttgttcaaaccaggcatttgggcactctcaatgttcaaattacttctaactaacataagaagttgcttgacaagttctgaaatcaataaatctaaaaattcaatacattacagaccaatctattagttgaccaacagaataaggaaataaatatcttaaggcataatagcaccagagctacactcaccttgacaaacaataggcaaatgccagcactgttgataatgattgtacaaaatttataagacgttgcttgacagcctggctagcttctgaaggtaggactacaggatctagtgctctgaaataaaccttgcagcagttagcgcagataaaatggctggaaacaagtttccagttaagataggtaaatagtgactctaaaatcattttcattccctgacttgtaatttgaatctaacttcttgtaatgtagaagtaaacctgcacataagagttgctccagtccaaagaagcaagggccggaggtaagaattcatgacatagtgcgacctacttttattccaagtactatcgttccgctgcagataatcaacatggaagaattaatcagctcaataacataatcaaaccgtgtctaaaggcaaggagtggcagaagaataacaaattatatacttgaagaaatataatcctgccaagacgcaaaagtggcccaagaccccaaacagcaaaagctatgatagcaactgctgggatcaatttaagtaaaacagggcttccacgtaaagcatcataagacctgcataatatgttaaaaacataaatttctttagataccaaacatttatataataatgaaagagaaaacatgatgaagcaattgccccccaataaatggaaaggaatacccaagaaaaagaacacaacttatatgctggcgtgtgcagctgactaaggaattcaaaagggaacgcgtatacctaccttgttaaaactacagcagcagatttcagaaggggaaccccatttcctccacctggtactaaaacagatctgcacaagaaaacattgcatctggaagatactggacctattgggccacgaacactgcttaaaagattgagactgcaggcaccctgtcgctgacaattaaaaagtaaataaataaataaatccatgagctcagatctacataatcacaacctcatcaggttacacacacacacacacacacacacacacacatccagagcatattgtaatatgctacactagaaccatgaacctgacaaaaacacgaaatgaagtacgatgagcaaatttttataacactagagcacaggcaacaatgccataaagggaaacggaaaaaaacaattaacaaaacaaaaacccagaaacccagtgacaattctgtataagaactcataccaaatcatagaccagcaattgaaaaaaatgataataaaataaagcataaaagtgaagccatgaaaccataccaaaacatgtggaggaaaattgattccgaccatatgtactcgaccctttcctacaaggcactgtgaaattcaaaaacaaaaccaaaaacaaaaaagcaaaaaaacaaagaattcaattagtactccagaaaaagtaaactcttgggcaagaagattttatgaaaaagactacaaagaaatctcacatacagcatctggattatggcatcgacggttactgtgacttctccattcatataacaaccgcacagaagcagtatggggcatggttgattgattctcaaactcaaactgaagcaaagctgcaactttgtgcacaatatgggaatgtagttgaaaatcagattacccttttgaacctaataacacactgtcttcaaattatgggtcttagactatatgcaccaattatgtacagcacctgtcacactaaaacttcaaattctcagttcatattgaaatctttcatacaaacaaacaaacaaacaaacaaataaataaatcgatttatagctaacaattaaagcagcagaacgatttttagataacttactaaatttttttacgctttttaagaagaaaacccatcatgaaaataaaatatttaaaaaaacaaattaatatgcaaaaagtttttaaaaattaaaataatatgaaaactgagaaataagtacggctttagttgaactaggtcagaggaacaagatttttcaaaattaaaaattactagaaattctgatttctaaggaaccaaaaccaaaaattaacaaggaaagtattgagtgttacctaatggagcaaagcagaggataaattcccaaactctctcacagaaactttcttgtagaggaaaccgggtttgagagagagagacagaggtgggggtactgtgaggaaggcaagcccaaaatggctgggtggatgctaagatacgccggttggtaaactatacacggcttcgcctgatagcgtggtcaaatcaaagtgtagcgttttggattttatttcgctacgtggacccgcgaaattacctttcggtttttcacggtgggatttatgtggcgcataggggtaaggtgcgcggtgtgcataattatttagcatcggtttcctccttgccagcattgcatggattggtctcgatcgatgatatttctgcggtgatcgggcggttgttctcaggtctgggtcaggtcaggttgggtcgggtttgattgatatacgcgataatggtttaaattgcaaagaaaagagggatttcccacacattcgaatctgaaaccactaatttacaaaccaagcttattttccactaggacagacctcgttagcaaatttgacttattgaaatagtttttcaacgaaactgtaatttgatcatacaaaatacggataatgatttgatcatacaaaattctgaaattttgttttttcattttattttataatacttttgttaaatatatgttattaatgtgtttgctttatggacttatggcgttacattatgaattgaatatgcatatttgaagaggcttgttacaatctgcctagttaatgtggaatggaaggaggcaaatgataggagtggaccgaaatattctcacactaggagaacgtgttcaaaacgtaacagaattcaaagtaatttaacatgttaggatatgaaaactacataaataaaattgaaactgttggcacaagagtagtaaaattaacaaaccacatgaataaaaagtgatttttttcctaataagatgttattatacttccaataaaaatactaatgaacataagtgttacgtgcataaacactcctaaacggtcatataatactttgagatatataaaaaatgcaagactcgcacattttttacgggcagtatataaaagttcaagctgaactggtgaactttatttttcgttgttgtttttgtgttaatcgaatttgaataaacaaaaatgagctgaccaaagataccttgaattactaatagggtgttagttggattggcatagacttttgatgtgctcttaagtggtcatgagttcaagtccctatggtatctgttcgtgcaaataatctcacgggatcggttcccaagggtcgatccccttgggtgttctgcccgtggggcagcttaggagattgagcttgtgaagattcctctacatcttgtcgccgcgtgttggtcctcgagcaggttggtgggcgtcgattatcatcctctgcctgcaaggagggtcgctcaggtcgacgtctttggtgtctcggacgtgactggctaactccttcctcggttcccaagggtcgatccccttgggtgccctgcccgtggggcagattaggagattgagcttggaaggatccctctacagcttgctgtcgcatgttggccctcgagcaggttggtgggcgtctgtcgttgcgctcgtcacgcgactgctcacgaactcgagctccttgtgggcccaagcgggcgtggacattagtctgtgggcctaagcgggcgtggacattagtctgtgggcctagcctctcgagcacattggtccttggttctagccccgaacggctcagagtacggctcgcggctgtctgcgtctcatctacttgatctcgatatctcctctgccggtttgcccttgtctgaccagcttgggacctctcgaattgctcgtcgacatgctggtgtgccctcatctcctcgtcacggggtccaaggttagggccttgattcaagttgatctgcctgagtagctggccaatcaggttgttctggtcatcgaccttctgtgtgagttgatcaacccgtgagttaaggtccacctgactgcgcgggtcaagtagagcgttgtgcatcgggcctaacggcatatgggctagggctccattagacgcgtacacaggtgcatatgtcgagtgtgatcgcagaggagggagagaatggatctgctccaagactgggatgacgttggagtagccatggggctgagcagcaaatccggtagttgagtggtgtggctgctgctgtgctgcgacggcaggacagcggggcagagcagctcatgtaatcggcgccggcaactgcaaggctgtggaggtcccggggtcatgtggcggctgttctgccattcctgcagctgaatgcggctgcaaagcactggaggacgactgcagggcgttagcagccacgattccgtgcggttgcattgaggtttccaccacgaaaccacgaggtaggcccgtcgtggtggcgttactcttcgtacgccttgtatcaaccatggttgtccgaaaaaatgtgtgaaagatggatttagagcacgctttgaatatatctcgtgctctcaatgaaagcaccaaatgtttgtgcaaataatctcacgggagaatattcgcttctagatccttcaaccttcgatcttccttctctctcttcctcgattcctgtaaaaaagactggagtaaatagaccacacccggggggtgttggccaaaggccctccgatgcctaagttagttcgagtgtttgtaggaaaacaatagctaagcaaagggtacggagttttatgtagggtgtaaaccgggtggccggagccgtgtgtggtggccggagccgtggggagaaaatatggagagtggagagggagagagagcttcaggtatttttcttgggtatcaggagtaggtttagatgtaccttgaatgatgaaggaggtcgtctatttataggagcctcggggctagggtttcgtaggaatcgagttgggcctgataatatatgaattaaatagatattatctcttaagaagataatatctgatttaaatgatattatcttctctttattaggataatatcttaattaatgatattatctctttaaataaagataatatcatattaattaagatatttatcccaattaattaattaaccagataaactgatctaattaattaattaaagagataatcttcttttccacgtagcgtgccctgattggagacgaaaatatatgctcctacaatatccgtgtgtgtgagttttcccccctcccctttttaactttggtaattaaaaaaagtaattacaattttggtcactgtggttagagtcgaatctaattttggttacccagccttcaattttccgattttgatcatcataaacatcgtcaacaaatcaagtctaatggaggtattctatcagtttcaatgcacgtgatacgtgtgtctatgggcaatattgctctttcacccctaaacctacggcagtgctatctctcactctcagatggccggcacttatgaaacccaaaagatatttgagattggtagtgttgatatccttcttggtctttagaacaagccgagcttgttcatcatttcatcgcacttcttcaaagccttgcatcccttcctcaccacctcctaatacccaaacgaccacaaaagcaatcaaatcactcaaatcaattacacaagaaaaagttggcctcgtcaccggatcgaatttgaaatctaggctcgtctgtttaatatgcacatgcagttgagataaagcatttattttcacctcaaacaaaaatataaatttattgtatttttctccaattcgcctcctttaatctcaaactttgtttcttgctaggttgagaagttgtagagagggaagaagttgtaaagagcaggagagaaagagctaggttggggtgaaagtacgcacacaacacgtgcgttgaaattgacggaatactccaattggacttgattgctgacgatgcaaagtgtgatgaccaaaatcgaaaaattgaaagttgggtgaccaaaattaaattcgaccctaacttcactggacaaaattgtaattactcaaaaaaaatataaattctaaaaatttatcgtttactattcggattagaggtcgtagaccatacattttggcggtgaatggactcgagcaatcctggtcaacgacttgcgtcaaagagaagtttagacgatggtccatagaaagccaccaacggagcacgccacagcttatcattttttccatgaatagtgatgatcaaacacgtttatggtgtgctgatgttgtttgcctattattttggataagattttatatatatatatatatatatatatatatattttttttttggggtatggataggtcatacgaccaaatttcataacccttttcgcaacttcagaaattgatattaccataatacccttctgccacttaatccatctttctgacgtctgaatgctaaactcttttctcactgtttctataacctctgccatttcggttttccatccacagcggcggaggctaatcaatttaaattttcgaccttatgaattttgcattcctttgtttcattgcaatttttatgtaagagcattcgtaatggttgctcaatagtttatttcagttcggtttaatagttcaatttattgaacttttttttgcagttattgaacaaaactgcagtttcacttgaaatggtgcacttcaatagcttacaaattaaaaattatatttaaaatatatatatatatatattaaaatgattgaataaaataaaatgaataaaggtaattacgataacaattatataattaaaatggtttcaaagatatctttaaaaagagacctattttgaatacatataaataaaaacttgtgaaaatcgatgatacttttggttaatatgaacccaaagatcatctaagtacctacgatcggattttgcaaaatttcaacataggttgtgcacagtaaattttcttgctccctggtataatgagttccctttttttggttgctcattttttattgaaaaataaaatttcaacttaaacatttttttttttgtgggtgtaggaaattgtgggagagttgttttctacgttgtatttataggtaaataactagccatttgaaaaggtaactaaccatttgtatttttgaaaagtgaaataaattagcaatagtatatataattagctatcctttttatgaaaagtgaaacaaatcaataatgactagtatatatattaattagatgttttttttaacagtgaaataaatcagaaacgactagttgctttgaaaaaataaaagtaacggctagtctacactaattaaatgtaatggaaaaattaaaaagtaatgttatccatgtgtcaataagagagaggctatagagttattatgttggccaatttattgaaatgaatttcatttcaaaatttattttagtgacttctgaaatgaactattgattcatgaaataaaatattaatctttgaaataaactaaactttaccactacaagtcaaaagttcaatttattaaaataaaattgaaatgaccctcaccatttatgagctatctaaaactagtgaattagatgtaagaaaaatggctaaatgagcacatgtgcacattgatagccatatggacgaacgtgtggattagagacttatgggttagaaagtgtttgtgtcagaatttgagtttttgcttatttctgaccaaaatacgtgatctagaagatttcaaggaaattacttattatgcatacaaaattgattttaaaaaagaaaaggaaaatcataataatattaccgtgattttattggataatttttatagaaattttctttgattacttggaaaacaagacaatattttacacaagcattgcaatccatgcaatacgtgagcttgccatattttgcctactatcttgcactgttgccgtacttgcataaatgcaatcaagggatgtgttgcactgtagctctagtggtagttctgcgggtgtcaatagtgtcccaactccctttccgtcctaataatttggaatgaattgacaccaaaaaaacaaacaaacaaacggagaacaatctacaccgaaaacttttcctttgtagtcaaattctttgccaattaggttatgtgacctatcacttaatttcttttgtcaatgaatttgaatttaaacattagcttcttaaagtgaattttttttcctttgtgagaaaaaaagaatggatgttaacaactttccacttataacattatcatgcatacaactattttttatggtttttgactcatgactataattcaaaactgataaattttcaccgaagtaaaacacaaaacagagctagttgttccagaaagcagcccccctgggcccaactcaaaccgcttctttaggaaaaacatttccaaaattatttcagtgggccaggctgttccgggatcactaccaatttccaaaattaaaacccgctacgccggagcacgtgccagtcacgtgacctccctatattttgaattcggacttgtccgccatctctcaaatctactttaatttgacccccgtccccctcccaattcaaattccctgcccctctctctctctctctctctctctctctctctctctctctctcgcctttcaaaccaaacagccaaaattgagagcaaatcccattctggtccaccttcttcaggtgcaactctctcagatccattaatatcttcctacttttcagctatcattgtctaatcgttttcaattattttttgctatttttgtctggtaatttttgtgcgaataatttggttcttacttctgattctgattagggattcatttcaatctggaggcgcgggggggaggatttaggattttcgatttcttgaataaagctttgctttaggtcgagatggatagttcggaatgtgttcgagtggctgtcaacataaggccattgattacctccgagcttctgataggctgcacagattgcatttctgttgttcccggtgaaccccaggtagctttttcatattggtagaaattttaaaaatttaaggtgaagttttggttttcatatttgggaatgtcaaatgtggctgattgagctatttattttgttgcatattaatttttgcatgtaaattttgtacatggatggcttattggtgcccctaagcttgggcccggatagaacatagtagagatatgagatgagaaggttctcttaattttcactcaagctaccaaaacatccaaatattactacttgaataaaatatgtgttcactatgtcactcttcattcttatttccaagtaaaatcctacaatctgattttacctttttataacttttagtttcatataatgactaacaaaccctgactttttatgttggtttttccaactttctcatattctatcttccaagctccaatcctttcaaaatcggggaaagaagaacttgggatcccatagattatttatccctcttttcttggcttttgtttttggtaaagaggtcacttctttcctatccaaccagtttcatggttgcttttttctcctttaccaggcatagtgtgtttagggtttcaatgatctttcggcatgaacttaatgaaaagtcatgattcaagtgagtaaatggtctaatcaaaaccctctgttggtgttagggggtgtttatgggacgaagctt